SEQUENCE LISTING (24 sequences)

SEQ ID NO: 1 - DNA, 1643 bp, Brassica napus
  5′UTR (1)..(42)
  CDS (43)..(1536)
  3′UTR (1537)..(1643)
agtacttatatatttttacattaagaaatctcccttccgaagatgtctcaagcaacgatgccagatttttctagctcggtgaagctcaagtatgtgaaacttggctatcaatacttagtcaatcacttcttgagtcttcttttgatccccatcatggcaatcatcgccgttgagcttcttcggatgggtccagaagatattcttaatgtttggaactcacttcactttgacatagttcaggtcctatgttcttctttccttgttatcttcatctccaccgtgtacttcatgtccaagccacgcaccgtctaccttgttgactactcttgttacaaaccgcctgtcacgtgccgtgtccccttcgcaactttcatggaacattcacgtttgatccttaaggactcgcctaagagcgttgagtttcaaatgcgaatccttgaacgttctggtctaggtgaagagacttgtctcccaccagctattcattacattcctccgacaccaaccatggacgaggcaagaagcgaggctcagatggttatcttcacggctatggatgatctcttcaagaaaactggtcttctgcctaaagacatcgacatactcattgtcaactgctctctcttctcgcccaccccatcgctctcagcaatggtcatcaacaaatataagcttaggagcaacattaaaagctttaatctctctgggatgggctgcagcgcgggcctgatctcagtcgatctggcccgtgatctccttcaggttcatcccaattctaatgcggtcattgtcagcaccgagatcataactcctaactactacaaagggaacgagagagccatgttgcttcccaactgtctcttccgcatgggatccgcggcaattcttatgtcaaaccgtcggtctgaccggtggagagccaagtacaagctttgccaccttgtccggactcaccgtggcgctgacgacaagtctttctactgcgtctatgaacaggaagatagagaaggaaaaattggcatcaacttgtccaaagatcttatggccatcgccggtgaagccctcaaggctaacatcaccacaataggtcctctggtcctaccagcatcagagcaactcctcttcctcttgtctttaatcggacgtaaaatctttaacctgaaatggaaaccatacatacctgatttcaagctagccttcgaacacttctgcatccatgcaggaggtagagccgtaatcgatgaactccaaaagaatctacagctatctggagaacacgtggaagcctcgagaatgacattacatcgttttggtaacacatcatcgtcatcgctgtggtacgagctttgctacatcgagtctaaagggagaatgaggagaggagatcgagtttggcagattgcgtttggaagtggtttcaagtgtaactctgctgtctggaaatgtaaccgtacgattaagacacctaaggatggaccatggtccgattgtatcgaccgttaccctgtctttatccctgaagttgttaaactttgaaccaaaacgtcttcaagtacggtttgtttacggtttggattttgtgattcggtttttaaggggattgggaaatggaggtttgaaataagtaaaaaaaaaaaaaaaaa

SEQ ID NO: 2 - protein, 497 aa, Brassica napus
MSQATMPDFSSSVKLKYVKLGYQYLVNHFLSLLLIPIMAIIAVELLRMGPEDILNVWNSLHFDIVQVLCSSFLVIFISTVYFMSKPRTVYLVDYSCYKPPVTCRVPFATFMEHSRLILKDSPKSVEFQMRILERSGLGEETCLPPAIHYIPPTPTMDEARSEAQMVIFTAMDDLFKKTGLLPKDIDILIVNCSLFSPTPSLSAMVINKYKLRSNIKSFNLSGMGCSAGLISVDLARDLLQVHPNSNAVIVSTEIITPNYYKGNERAMLLPNCLFRMGSAAILMSNRRSDRWRAKYKLCHLVRTHRGADDKSFYCVYEQEDREGKIGINLSKDLMAIAGEALKANITTIGPLVLPASEQLLFLLSLIGRKIFNLKWKPYIPDFKLAFEHFCIHAGGRAVIDELQKNLQLSGEHVEASRMTLHRFGNTSSSSLWYELCYIESKGRMRRGDRVWQIAFGSGFKCNSAVWKCNRTIKTPKDGPWSDCIDRYPVFIPEVVKL

SEQ ID NO: 3 - DNA, 1494 bp, Brassica napus
  CDS (1)..(1494)
atgtctcaagcaacgatgccagatttttctagctcggtgaagctcaagtatgtgaaacttggctatcaatacttagtcaatcacttcttgagtcttcttttgatccccatcatggcaatcatcgccgttgagcttcttcggatgggtccagaagatattcttaatgtttggaactcacttcactttgacatagttcaggtcctatgttcttctttccttgttatcttcatctccaccgtgtacttcatgtccaagccacgcaccgtctaccttgttgactactcttgttacaaaccgcctgtcacgtgccgtgtccccttcgcaactttcatggaacattcacgtttgatccttaaggactcgcctaagagcgttgagtttcaaatgcgaatccttgaacgttctggtctaggtgaagagacttgtctcccaccagctattcattacattcctccgacaccaaccatggacgaggcaagaagcgaggctcagatggttatcttcacggctatggatgatctcttcaagaaaactggtcttctgcctaaagacatcgacatactcattgtcaactgctctctcttctcgcccaccccatcgctctcagcaatggtcatcaacaaatataagcttaggagcaacattaaaagctttaatctctctgggatgggctgcagcgcgggcctgatctcagtcgatctggcccgtgatctccttcaggttcatcccaattctaatgcggtcattgtcagcaccgagatcataactcctaactactacaaagggaacgagagagccatgttgcttcccaactgtctcttccgcatgggatccgcggcaattcttatgtcaaaccgtcggtctgaccggtggagagccaagtacaagctttgccaccttgtccggactcaccgtggcgctgacgacaagtctttctactgcgtctatgaacaggaagatagagaaggaaaaattggcatcaacttgtccaaagatcttatggccatcgccggtgaagccctcaaggctaacatcaccacaataggtcctctggtcctaccagcatcagagcaactcctcttcctcttgtctttaatcggacgtaaaatctttaacctgaaatggaaaccatacatacctgatttcaagctagccttcgaacacttctgcatccatgcaggaggtagagccgtaatcgatgaactccaaaagaatctacagctatctggagaacacgtggaagcctcgagaatgacattacatcgttttggtaacacatcatcgtcatcgctgtggtacgagctttgctacatcgagtctaaagggagaatgaggagaggagatcgagtttggcagattgcgtttggaagtggtttcaagtgtaactctgctgtctggaaatgtaaccgtacgattaagacacctaaggatggaccatggtccgattgtatcgaccgttaccctgtctttatccctgaagttgttaaactttga

SEQ ID NO: 4 - protein, 497 aa, Brassica napus; identical to SEQ ID NO: 2

SEQ ID NO: 5 - DNA, 1699 bp, Brassica napus
  5′UTR (1)..(30)
  CDS (31)..(1563)
  3′UTR (1564)..(1699)
cgtcagttcaacaaatctcatctctcaaccatggacggtgccggaggaagcagccgtgacggttcagtcggagtacagatccgacaaacccggatcctaccggactttctccagagcgtgaatctcaagtacgtgaagctaggctaccattacctaatctcgaatctcctggctctctgtctcctccccctcgccgtcgttatctccgtcgaagcgtctcagaccgacccggaagatctccgccagctctggatccatctccagtacaatctcgtcagcatcatcgcctgctccgccgtccttgtcttcggtttaaccgtctacgtaatgacccggcccaggcccatttacttggtggacttctcctgttacctcccgcctgaccgcctcaaggttcccgtctctcgcttcatgaaccactctacactcaccggagacttcgacgagtcctccctcgagttccagcgcaagatcctcgagcggtctggtctgggagaggacacgtacctccccgaggctatgcaccacgtcccgcccacgatctcgatggccgccgctagagaagaagctgagcaggtcatgttcggcgcgttggataagctgttcacgaacacgaacgtgaaggcgaaggatattggggtcttggttgtgaactgtagtctctttaacccgacgccttcgctgtcggcgatgatcgtgaataagtataagcttaggggtaacatcagaagctacaacctcggcgggatgggatgcagcgctggggtcatcgcggtggatctcgctaaagatatgttgttagtgcataggaacacttacgcggttgttgtctccacggagaacattactcagaactggtacttcggtaacaagaaagcaatgttgattcctaactgcttgttccgcgtcggaggctccgcggttctgctgtccaacaagtctagggacaagagaaggtccaagtacaggctttcgcacgtggtcaggacacaccgtggagcagacgacaaggctttccgttgcgtgtatcaagaacaggacgacacgaggagaaccggtgtgtccttgtcgaaagagctgatggctattgctggggagactctcaagactaatatcaccacgttgggtcctcttgtgttaccgataagcgagcagattctcttcttcctgactcttgttgtgaagaagctttttaacggtaaggttaaaccgtatattccggatttcaaacttgcgttcgagcatttctgtatccatgctggtggaagagctgtgatagatgagttggagaagaatctgcagctttcgccggttcatgtcgaggcttcgaggatgactcttcataggttcgggaacacgtcttctagctccatttggtatgagctggcttacattgaggctaaaggaaggatgaggagaggcaaccgggtttggcagattgcgtttgggagtgggtttaagtgtaacagtgcggtttgggaagctttgaggaatgtgaaaccgtctaaggacagtccttgggaagattgtattgataagtatcctgtaaccttaagttattaggctttgtttgagggaattggagagactaatcagtacccatggttgtgttctgtttttggtctttggcaaatgtcaatgttcttgttgtttttataatgttttgggagttttttgtgataaaaaaaaaaaaaaaaaa

SEQ ID NO: 6 - protein, 510 aa, Brassica napus
MDGAGGSSRDGSVGVQIRQTRILPDFLQSVNLKYVKLGYHYLISNLLALCLLPLAVVISVEASQTDPEDLRQLWIHLQYNLVSIIACSAVLVFGLTVYVMTRPRPIYLVDFSCYLPPDRLKVPVSRFMNHSTLTGDFDESSLEFQRKILERSGLGEDTYLPEAMHHVPPTISMAAAREEAEQVMFGALDKLFTNTNVKAKDIGVLVVNCSLFNPTPSLSAMIVNKYKLRGNIRSYNLGGMGCSAGVIAVDLAKDMLLVHRNTYAVVVSTENITQNWYFGNKKAMLIPNCLFRVGGSAVLLSNKSRDKRRSKYRLSHVVRTHRGADDKAFRCVYQEQDDTRRTGVSLSKELMAIAGETLKTNITTLGPLVLPISEQILFFLTLVVKKLFNGKVKPYIPDFKLAFEHFCIHAGGRAVIDELEKNLQLSPVHVEASRMTLHRFGNTSSSSIWYELAYIEAKGRMRRGNRVWQIAFGSGFKCNSAVWEALRNVKPSKDSPWEDCIDKYPVTLSY

SEQ ID NO: 7 - DNA, 1533 bp, Brassica napus
  CDS (1)..(1533)
atggacggtgccggaggaagcagccgtgacggttcagtcggagtacagatccgacaaacccggatcctaccggactttctccagagcgtgaatctcaagtacgtgaagctaggctaccattacctaatctcgaatctcctggctctctgtctcctccccctcgccgtcgttatctccgtcgaagcgtctcagaccgacccggaagatctccgccagctctggatccatctccagtacaatctcgtcagcatcatcgcctgctccgccgtccttgtcttcggtttaaccgtctacgtaatgacccggcccaggcccatttacttggtggacttctcctgttacctcccgcctgaccgcctcaaggttcccgtctctcgcttcatgaaccactctacactcaccggagacttcgacgagtcctccctcgagttccagcgcaagatcctcgagcggtctggtctgggagaggacacgtacctccccgaggctatgcaccacgtcccgcccacgatctcgatggccgccgctagagaagaagctgagcaggtcatgttcggcgcgttggataagctgttcacgaacacgaacgtgaaggcgaaggatattggggtcttggttgtgaactgtagtctctttaacccgacgccttcgctgtcggcgatgatcgtgaataagtataagcttaggggtaacatcagaagctacaacctcggcgggatgggatgcagcgctggggtcatcgcggtggatctcgctaaagatatgttgttagtgcataggaacacttacgcggttgttgtctccacggagaacattactcagaactggtacttcggtaacaagaaagcaatgttgattcctaactgcttgttccgcgtcggaggctccgcggttctgctgtccaacaagtctagggacaagagaaggtccaagtacaggctttcgcacgtggtcaggacacaccgtggagcagacgacaaggctttccgttgcgtgtatcaagaacaggacgacacgaggagaaccggtgtgtccttgtcgaaagagctgatggctattgctggggagactctcaagactaatatcaccacgttgggtcctcttgtgttaccgataagcgagcagattctcttcttcctgactcttgttgtgaagaagctttttaacggtaaggttaaaccgtatattccggatttcaaacttgcgttcgagcatttctgtatccatgctggtggaagagctgtgatagatgagttggagaagaatctgcagctttcgccggttcatgtcgaggcttcgaggatgactcttcataggttcgggaacacgtcttctagctccatttggtatgagctggcttacattgaggctaaaggaaggatgaggagaggcaaccgggtttggcagattgcgtttgggagtgggtttaagtgtaacagtgcggtttgggaagctttgaggaatgtgaaaccgtctaaggacagtccttgggaagattgtattgataagtatcctgtaaccttaagttattag

SEQ ID NO: 8 - protein, 510 aa, Brassica napus; identical to SEQ ID NO: 6

SEQ ID NO: 9 - DNA, 2036 bp, Glycine max
  5′UTR (1)..(219)
  3′UTR (1786)..(2036)
  CDS (220)..(1785)
atttctctcccttttctaatttattttctaagcccaacacatacaaccttatattctcccattcatcataaccaaaaactttgtcactggcttatttattctctattaatattatatcctttgggtattgtattgtatcgtattgctttatacaaattcttaactcaattagcctagctagctaggtcattataaaaaaaattaaaatagtgttccttgatggctgaccagaagaatcaaaacccggattcaggaaccattagtgttgaatcagcaaaagaaaaaagaaggaacaaacttcccaacttcctgctatcagtgagactcaagtatgtgaagctagggtaccattacgtaatctccaatgcaatgtatcttctgctcataccccttctaggcatagcttcggctcatctttcaaccctttccatcaaggactttctccaattatgggagaatctcaagttcaaccttgtctcggtaaccctatgttcaagcctcatggtgttcctcgccaccctctacttcatgacccgtccaaggggggtgtacttagtagattttgcatgctacaagcctgacgtggattgcacgtgcacgcgggagattttcgtggagaggtctggtctcacaggctcattctcggaggagaacttgtcctttcagaagaagatccttgagaggtctggtttggggcagaagacgtatcttccgcccgcaatcttgagtttgccgccgcgaccgtgcatggcggcggccagggaggaggctgagcaagtcatgtttggagcaattgaccagctgctggcaaagactggtgtgaaggctaaggatattgggatcttggtggtcaattgcagcttgttcaaccccacaccttcactctctgccatgattgtcaatcactacaagttgagagggaatgttttcagttataatcttggtggcatgggttgcagtgctggccttatctctattgaccttgccaagcacctcctccaggtccatcccaactcttatgccttggtagtgagcatggagaacatcactctgaactggtattttggcaacaaccggtcaatgctagtctcaaactgtctcttcagaatgggaggagcagcaatcctcctctccaaccgctcgggcgaccgccaccgcgccaaataccaactagtccacacagtgcggactcacaaaggagcagatgacaagtcctacggctgtgtcttccaagaagaagatgagacaaaaagaattggtgtggcactctcaaaagacctaatggctgtggcaggagaggccctaaagaccaacatcacaacactaggacccttggtcctccctatgtcagaacagcttcttttctttgccacattggtggctaggaaagtgttcaagatgaagataaaaccatacatcccagatttcaagttggcctttgagcatttttgcattcatgctggagggagggcagtgttggatgagttggagaagaatcttgagctctctgattggcacatggagccctcaaggatgacactaaataggtttggtaacacttctagcagttccttgtggtatgaattggcctacactgaagccaaagggaggatcaagaaaggtgacaggacttggcagattgcatttgggtcagggtttaagtgcaacagtgctgtgtggagggctttgaggaccatcaatcctgctaaggagaaaaatccttggatggatgagattcatgagtttcctgttcatgtgcctaaagtggcaccaattgcttcctaaattaatcaaacatctttttctctttttagtacgattctaaaattaaagaaacttgttcatgaaacagaagagaagattgttaattattactcctgtagcttttccagtttctttggtcttgtaacattgtgttgaatttaaaagagtgtgcatacagttactcccctgttatttatttcattatttccaataaatgtatcaattgaccacctgaaggtgttcaaattttctacaaaaaaaaaaaaaaaaaa

SEQ ID NO: 10 - protein, 521 aa, Glycine max
MADQKNQNPDSGTISVESAKEKRRNKLPNFLLSVRLKYVKLGYHYVISNAMYLLLIPLLGIASAHLSTLSIKDFLQLWENLKFNLVSVTLCSSLMVFLATLYFMTRPRGVYLVDFACYKPDVDCTCTREIFVERSGLTGSFSEENLSFQKKILERSGLGQKTYLPPAILSLPPRPCMAAAREEAEQVMFGAIDQLLAKTGVKAKDIGILVVNCSLFNPTPSLSAMIVNHYKLRGNVFSYNLGGMGCSAGLISIDLAKHLLQVHPNSYALVVSMENITLNWYFGNNRSMLVSNCLFRMGGAAILLSNRSGDRHRAKYQLVHTVRTHKGADDKSYGCVFQEEDETKRIGVALSKDLMAVAGEALKTNITTLGPLVLPMSEQLLFFATLVARKVFKMKIKPYIPDFKLAFEHFCIHAGGRAVLDELEKNLELSDWHMEPSRMTLNRFGNTSSSSLWYELAYTEAKGRIKKGDRTWQIAFGSGFKCNSAVWRALRTINPAKEKNPWMDEIHEFPVHVPKVAPIAS

SEQ ID NO: 11 - DNA, 1566 bp, Glycine max
  CDS (1)..(1566)
atggctgaccagaagaatcaaaacccggattcaggaaccattagtgttgaatcagcaaaagaaaaaagaaggaacaaacttcccaacttcctgctatcagtgagactcaagtatgtgaagctagggtaccattacgtaatctccaatgcaatgtatcttctgctcataccccttctaggcatagcttcggctcatctttcaaccctttccatcaaggactttctccaattatgggagaatctcaagttcaaccttgtctcggtaaccctatgttcaagcctcatggtgttcctcgccaccctctacttcatgacccgtccaaggggggtgtacttagtagattttgcatgctacaagcctgacgtggattgcacgtgcacgcgggagattttcgtggagaggtctggtctcacaggctcattctcggaggagaacttgtcctttcagaagaagatccttgagaggtctggtttggggcagaagacgtatcttccgcccgcaatcttgagtttgccgccgcgaccgtgcatggcggcggccagggaggaggctgagcaagtcatgtttggagcaattgaccagctgctggcaaagactggtgtgaaggctaaggatattgggatcttggtggtcaattgcagcttgttcaaccccacaccttcactctctgccatgattgtcaatcactacaagttgagagggaatgttttcagttataatcttggtggcatgggttgcagtgctggccttatctctattgaccttgccaagcacctcctccaggtccatcccaactcttatgccttggtagtgagcatggagaacatcactctgaactggtattttggcaacaaccggtcaatgctagtctcaaactgtctcttcagaatgggaggagcagcaatcctcctctccaaccgctcgggcgaccgccaccgcgccaaataccaactagtccacacagtgcggactcacaaaggagcagatgacaagtcctacggctgtgtcttccaagaagaagatgagacaaaaagaattggtgtggcactctcaaaagacctaatggctgtggcaggagaggccctaaagaccaacatcacaacactaggacccttggtcctccctatgtcagaacagcttcttttctttgccacattggtggctaggaaagtgttcaagatgaagataaaaccatacatcccagatttcaagttggcctttgagcatttttgcattcatgctggagggagggcagtgttggatgagttggagaagaatcttgagctctctgattggcacatggagccctcaaggatgacactaaataggtttggtaacacttctagcagttccttgtggtatgaattggcctacactgaagccaaagggaggatcaagaaaggtgacaggacttggcagattgcatttgggtcagggtttaagtgcaacagtgctgtgtggagggctttgaggaccatcaatcctgctaaggagaaaaatccttggatggatgagattcatgagtttcctgttcatgtgcctaaagtggcaccaattgcttcctaa

SEQ ID NO: 12 - protein, 521 aa, Glycine max; identical to SEQ ID NO: 10

SEQ ID NO: 13 - DNA, 1944 bp, Glycine max
  5′UTR (1)..(74)
  CDS (75)..(1682)
  3′UTR (1683)..(1944)
agaatactcataataatatagtggctagcatagagctagtaataatattaatattgttaagctagctctaagccatggctaatgaacgtgatctgttctcggcggagattgtgaaccgtggaattgaatcgtcaggaccaaacgctgggtcgttgacgttttcggtgagggtgagaaggcgcttaccggattttcttcaatcggtgaaccttaagtacgtgaaattgggttaccattatctgataaaccatggcatatacttgttcaccataccggttcttctcgtggtgtttagtgctgaggttggaagcctcagcaaagaggacctatggaagaagctttgggaagacgcgcgttacgaccttgctacggttcttgcttcctttggggtcttcgttttcaccttgtctgtctacttcatgtcgaggccacgtcccatttacctcattgattttgcatgttataaacccgatgatgaactcaaggtgtcaagggagcagttgatggaggtagcaagaaaatcaggcaagttcgacgaagcgagtctggagttccagaagaggatgctgatgtcctccggaatcggcgacgagacctacatcccgaaggccgtcgtggcctccacggagaacacggcgacaatgaaagaggggcgcggcgaggcctccatggtgatgttcggagccctggacgagctgttcgagaaaagccgcgtccgaccaaaggacgtgggcgtcctagtagtgaactgcagcatcttcaacccgacaccatcactctcagcgatgatcataaaccactacaagatgagagggaacattctgagctacaacctcggcggaatggggtgcagcgcggggatcattggtgtggacttggccaaggatattctccaggcgaaccctaataactacgcggtggtggtgagcacggagatggtggggtacaactggtaccaggggaaggacaggtctatgcttatcccgaatagcttcttccgcatggggtgttctgctgttctgctttccaaccgtcgaagggactatagccgcgcaaagtaccgtcttgaacacattgttcgcacgcataagggtgccgatgaccgcagcttcaggtgtgtttaccaagaggaagatgagcaaaaactcaagggactaaagatcagcaaagacttgattgagattggtggtgatgctctaaaaaccaacatcaccacactaggacccttggtgctacccttttctgagcagcttctcttctttgccacccttgtgtggaggcacttatttggcagcaaaaatggtgggaattcaccctcaatgaagaagccttacattcctgactacaaactagcttttgagcacttttgtgtgcatgctgcaagcaagaccattcttgatgagcttcaaaggaacttggaactgagtgacaagaacatcgaagcgtctagaatgacgctgcatcgctttggcaacacctctagcagcagcatttggtatgagttggcttacatggaagcaaaagagagtgttagaagaggagaccgtgtttggcaactcgcatttggctctggcttcaagtgcaacagtgtggtttggcgcgccatgcgccgagtcaccaagccttctaggaacccttggcttgattgcatcaacagatatcctgcacctctcaacaactgaagcagaatttgacggcactagtgttatgaatcttattatcagtgagggtatggttaggatcaataagggtagtactaatttggtcttcaaaaatgttacatctgttttctgtttctttctcatttaattattcctggcttgaaattctatacttttaattcctcattaggcatatggatgcctccttgtttaatttctctgtttcttcataggatgattgatatcaaagttcatggatggctattaccgaaaaaaaaaaaaa

SEQ ID NO: 14 - protein, 535 aa, Glycine max
MANERDLFSAEIVNRGIESSGPNAGSLTFSVRVRRRLPDFLQSVNLKYVKLGYHYLINHGIYLFTIPVLLVVFSAEVGSLSKEDLWKKLWEDARYDLATVLASFGVFVFTLSVYFMSRPRPIYLIDFACYKPDDELKVSREQLMEVARKSGKFDEASLEFQKRMLMSSGIGDETYIPKAVVASTENTATMKEGRGEASMVMFGALDELFEKSRVRPKDVGVLVVNCSIFNPTPSLSAMIINHYKMRGNILSYNLGGMGCSAGIIGVDLAKDILQANPNNYAVVVSTEMVGYNWYQGKDRSMLIPNSFFRMGCSAVLLSNRRRDYSRAKYRLEHIVRTHKGADDRSFRCVYQEEDEQKLKGLKISKDLIEIGGDALKTNITTLGPLVLPFSEQLLFFATLVWRHLFGSKNGGNSPSMKKPYIPDYKLAFEHFCVHAASKTILDELQRNLELSDKNIEASRMTLHRFGNTSSSSIWYELAYMEAKESVRRGDRVWQLAFGSGFKCNSVVWRAMRRVTKPSRNPWLDCINRYPAPLNN

SEQ ID NO: 15 - DNA, 1608 bp, Glycine max
  CDS (1)..(1608)
atggctaatgaacgtgatctgttctcggcggagattgtgaaccgtggaattgaatcgtcaggaccaaacgctgggtcgttgacgttttcggtgagggtgagaaggcgcttaccggattttcttcaatcggtgaaccttaagtacgtgaaattgggttaccattatctgataaaccatggcatatacttgttcaccataccggttcttctcgtggtgtttagtgctgaggttggaagcctcagcaaagaggacctatggaagaagctttgggaagacgcgcgttacgaccttgctacggttcttgcttcctttggggtcttcgttttcaccttgtctgtctacttcatgtcgaggccacgtcccatttacctcattgattttgcatgttataaacccgatgatgaactcaaggtgtcaagggagcagttgatggaggtagcaagaaaatcaggcaagttcgacgaagcgagtctggagttccagaagaggatgctgatgtcctccggaatcggcgacgagacctacatcccgaaggccgtcgtggcctccacggagaacacggcgacaatgaaagaggggcgcggcgaggcctccatggtgatgttcggagccctggacgagctgttcgagaaaagccgcgtccgaccaaaggacgtgggcgtcctagtagtgaactgcagcatcttcaacccgacaccatcactctcagcgatgatcataaaccactacaagatgagagggaacattctgagctacaacctcggcggaatggggtgcagcgcggggatcattggtgtggacttggccaaggatattctccaggcgaaccctaataactacgcggtggtggtgagcacggagatggtggggtacaactggtaccaggggaaggacaggtctatgcttatcccgaatagcttcttccgcatggggtgttctgctgttctgctttccaaccgtcgaagggactatagccgcgcaaagtaccgtcttgaacacattgttcgcacgcataagggtgccgatgaccgcagcttcaggtgtgtttaccaagaggaagatgagcaaaaactcaagggactaaagatcagcaaagacttgattgagattggtggtgatgctctaaaaaccaacatcaccacactaggacccttggtgctacccttttctgagcagcttctcttctttgccacccttgtgtggaggcacttatttggcagcaaaaatggtgggaattcaccctcaatgaagaagccttacattcctgactacaaactagcttttgagcacttttgtgtgcatgctgcaagcaagaccattcttgatgagcttcaaaggaacttggaactgagtgacaagaacatcgaagcgtctagaatgacgctgcatcgctttggcaacacctctagcagcagcatttggtatgagttggcttacatggaagcaaaagagagtgttagaagaggagaccgtgtttggcaactcgcatttggctctggcttcaagtgcaacagtgtggtttggcgcgccatgcgccgagtcaccaagccttctaggaacccttggcttgattgcatcaacagatatcctgcacctctcaacaactga

SEQ ID NO: 16 - protein, 535 aa, Glycine max; identical to SEQ ID NO: 14

SEQ ID NO: 17 - DNA, 2514 bp, Glycine max
  5′UTR (1)..(152)
  CDS (153)..(1685)
  3′UTR (1686)..(2514)
ttcatattcatacatttatttaatcactacaattattcctcctcacataactgtttttctttctttctttcgtccctctgctctaccatcaacgtctctctcttttcgggatacattcctgggatctgctggctttgtttttcaagggcaaaatgacagatacaaagccagatacacccttaacggcatcttcatcgcgaaaccttcctgatttcaagaagtctgttaagttgaagtatgtcaagcttggttatcattacctcatcacccatggaatgtacctttgcctttccccccttgtcgtattgattgcagcccagctttccaccttctccctccaagacttgtatgatctttgggagcatctacaatacaacttgatatctgttattctttgctcaactctccttgttttcctgtccaccctctactttctaactcgtcctcgaccagtgttccttgtcaatttctcctgttacaagcctgaagaatctcggaagtgtgcgaaaaggatatttattgatcagtcccggttgaccggttcgttcacagaggaaaatcttgaattccagcggaagattcttgagagatctggccttggggagaacacttaccttcctgaggctgtcctcaacattcctcccaacccttcaatgaaagaagctagaaaagaagccgaggctgtgatgtttggtgccattgatgaactattagctaaaacctctgtaaagcctaaagacattggaattctgattgtaaattgtagtctgttcaaccctactccatcactttcggcaatgattgtcaatcattacaagctgcggggaaatataaagagctacaaccttggtgggatgggttgcagtgcagggctgatctcaattgatcttgctaaagatcttctccaagccaatcccaactcctatgcattggtcattagcatggagaatatcacgttgaattggtattttggaaacgatcggtcaaagcttgtctctaattgtttgtttcgcatgggaggagctgcagttctgctctccaataaaagctcggatagaagaagatccaaataccgattggtcaccacggttcgcactcataagggtgctgatgacaagtgctttagctgtgtaacccaagaagaagatgccaatggcaaggttggtgttactttgtcaaaagatctgatggctgttgctggtgatgctttgaaaaccaatatcactacactggggcctctcgtacttccaacatctgaacaactgctattctttgccacgttagttgcaaaaaagattttcaagatgaagatcaaaccttatattccagatttcaagctagcttttgaacatttttgcatccatgctggtggtagagctgttttggatgaactggagaaaaacttgcagctatctccttggcatatggagccttcaaggatgacgctctatcgttttggaaacacctccagcagttcactttggtatgaactggcttacactgaagccaaagggaggatcaaaaggggagatagaacatggcaaattgcatttggttctggattcaagtgtaacagtgcagtgtggaaggctctcaggaccatcaacccttctaaggagaagagtccttggattgatgagattgaccagtttccagtcgatgttcctagggtatcttccatttaaacaatgaattttttaaatatttgtgtatcactatcagcattaggtggtagctgttggactggtgaaattatcctgcaaagttgttgtaatacattgctaatttcggagtactgtaggattttatttttaggacaaaacttagatgccatgcagttcataagatgtttttgatattatattctaatcaaaattgaagtttcacctcggtaacttgtgttgatattcaatacaaatctttattatgcagattaccgagattaaacttcaattttaattagaaaacaataccaaaaacactttagaaagtgcatctctaagttttatcctttttattatggcatcctgcgtattagtgtgtgtctggtttgttggcagcatgcactccgagaccattgaacggagaagtatgtgagagagaactttgaaatgaacagacggttgggatggttgaacggataaccaaacacacacttagtagatctaacttgtgggtagtttttgttaaatccaatctagtcaatggtgggtgtttgatctttggttgccgggaggtgtgtttcccattggtctgtggtactaaaatgcgtagcgcatctaaaatgtatttccaaatttctctaatgaaattggaattgaacaattccatttcaattctttagtgtgttgatgctgaaaataaaattccaaggtatattattgcattgtaatttgtatcaccatctgtaacaattcatgatctatatgaggtgatttttagcttcatggctataccaggtatctgaaatatatagctcttacattagtgtattgcaaaaaaaaaaaaaaaaaa

SEQ ID NO: 18 - protein, 510 aa, Glycine max
MTDTKPDTPLTASSSRNLPDFKKSVKLKYVKLGYHYLITHGMYLCLSPLVVLIAAQLSTFSLQDLYDLWEHLQYNLISVILCSTLLVFLSTLYFLTRPRPVFLVNFSCYKPEESRKCAKRIFIDQSRLTGSFTEENLEFQRKILERSGLGENTYLPEAVLNIPPNPSMKEARKEAEAVMFGAIDELLAKTSVKPKDIGILIVNCSLFNPTPSLSAMIVNHYKLRGNIKSYNLGGMGCSAGLISIDLAKDLLQANPNSYALVISMENITLNWYFGNDRSKLVSNCLFRMGGAAVLLSNKSSDRRRSKYRLVTTVRTHKGADDKCFSCVTQEEDANGKVGVTLSKDLMAVAGDALKTNITTLGPLVLPTSEQLLFFATLVAKKIFKMKIKPYIPDFKLAFEHFCIHAGGRAVLDELEKNLQLSPWHMEPSRMTLYRFGNTSSSSLWYELAYTEAKGRIKRGDRTWQIAFGSGFKCNSAVWKALRTINPSKEKSPWIDEIDQFPVDVPRVSSI

SEQ ID NO: 19 - DNA, 1533 bp, Glycine max
  CDS (1)..(1533)
atgacagatacaaagccagatacacccttaacggcatcttcatcgcgaaaccttcctgatttcaagaagtctgttaagttgaagtatgtcaagcttggttatcattacctcatcacccatggaatgtacctttgcctttccccccttgtcgtattgattgcagcccagctttccaccttctccctccaagacttgtatgatctttgggagcatctacaatacaacttgatatctgttattctttgctcaactctccttgttttcctgtccaccctctactttctaactcgtcctcgaccagtgttccttgtcaatttctcctgttacaagcctgaagaatctcggaagtgtgcgaaaaggatatttattgatcagtcccggttgaccggttcgttcacagaggaaaatcttgaattccagcggaagattcttgagagatctggccttggggagaacacttaccttcctgaggctgtcctcaacattcctcccaacccttcaatgaaagaagctagaaaagaagccgaggctgtgatgtttggtgccattgatgaactattagctaaaacctctgtaaagcctaaagacattggaattctgattgtaaattgtagtctgttcaaccctactccatcactttcggcaatgattgtcaatcattacaagctgcggggaaatataaagagctacaaccttggtgggatgggttgcagtgcagggctgatctcaattgatcttgctaaagatcttctccaagccaatcccaactcctatgcattggtcattagcatggagaatatcacgttgaattggtattttggaaacgatcggtcaaagcttgtctctaattgtttgtttcgcatgggaggagctgcagttctgctctccaataaaagctcggatagaagaagatccaaataccgattggtcaccacggttcgcactcataagggtgctgatgacaagtgctttagctgtgtaacccaagaagaagatgccaatggcaaggttggtgttactttgtcaaaagatctgatggctgttgctggtgatgctttgaaaaccaatatcactacactggggcctctcgtacttccaacatctgaacaactgctattctttgccacgttagttgcaaaaaagattttcaagatgaagatcaaaccttatattccagatttcaagctagcttttgaacatttttgcatccatgctggtggtagagctgttttggatgaactggagaaaaacttgcagctatctccttggcatatggagccttcaaggatgacgctctatcgttttggaaacacctccagcagttcactttggtatgaactggcttacactgaagccaaagggaggatcaaaaggggagatagaacatggcaaattgcatttggttctggattcaagtgtaacagtgcagtgtggaaggctctcaggaccatcaacccttctaaggagaagagtccttggattgatgagattgaccagtttccagtcgatgttcctagggtatcttccatttaa

SEQ ID NO: 20 - protein, 510 aa, Glycine max; identical to SEQ ID NO: 18

SEQ ID NO: 21 - DNA, 1693 bp, Linum usitatissimum
  5′UTR (1)..(21)
  CDS (22)..(1533)
  3′UTR (1534)..(1693)
ccgacacgcagcggccctgccatggactccactgccggcgagattcgaatccaacaaccccgccgcctacccgacttcctgcagagcgtaaacctcaaatacgtcaagctcggctaccactacctcatctccaatctacctaccttattcttcgtccctctaatcgccttaaccttaatcgaagcctcccagatggatcccgatgacctccgccacctctggctccacctccaatacaacctcgtcgccgtcatcacctgctccgccttcctcgtcttcggtctcaccgtctacatcgtcactcgacctgtccccgtctacctcctcgactacgcctgctaccgccctcccaaaaccctaatggctccttttgcccgcttcatggagcattcccgtctcactggcgacttcgacgagtcttccctcgagtttcagcgcaagatcctcgagcgatccggactcggcgaggagacttacgtccccgaggcgatgcacttcgtcccaccgcggccgacgatggctgccgccagggaggaggctgagcaggtaatgttcggtgccttggatactctattcgccaatgccaatgtaaaccctaaggatattgggattttggtagtcaattgtagcctgttcaatcccactccttccctctcggcgatgattgttaataagtacaagctccgtgggaatatccggagctttaatttgggtggtatggggtgtagcgccggtgtaattgcaatcgatttggccaaggatcttcttcaaatccaccggaaaacttgcgctgtcgtcgtgagtactgagaacattactcagaactggtactttgggaacaaaaaatcgatgctgatccctaattgcttgttcagagtgggtggatcggcagtcctgctctcgaataaatcgagtgataggaggagagccaagtacaagctcgtccatgtcgtacggactcacagaggagcagacgacaaggcttttcgctgcgtttaccaggagcaggatcccaatgggaagactggggtttcattgtcaaaggatttgatggcgattgcaggggaggcattgaaggcaaacattacgactctgggtcctctggtccttcctattagtgagcagcttctcttcttctcgacattggtcgtgaagaagctgttcaacaacaaggtgaagccgtatatcccagatttcaagctcgcattcgaacatttctgtatccacgcaggcgggagggctgtgatcgatgagctggagaagaatttgcagctgtctcttgttcatgtggaagcatcaaggatgactcttcataggtttggcaacacctcgtcgagttcaatctggtacgaattgggctacattgaggcgaaagggaggatgaggaaagggaatcgggtgtggcagattgcgtttgggagtgggtttaagtgtaacagcgctgtgtggcaggcgctgaggaatgtgaatccctcgccaaacggaccgtgggaagattgcatagataagtacccagttaagataatgtcttgacaataataatgtactatttaaggtatggaagtagctggtaactttaaagaggaggtatatgatatagctgcttgtcctgtttctttctactttgtgatccgagatagttggagtcaatgttagattgatgggagtgactaccaaaaaaaaaaaaaaaaaa

SEQ ID NO: 22 - protein, 503 aa, Linum usitatissimum
MDSTAGEIRIQQPRRLPDFLQSVNLKYVKLGYHYLISNLPTLFFVPLIALTLIEASQMDPDDLRHLWLHLQYNLVAVITCSAFLVFGLTVYIVTRPVPVYLLDYACYRPPKTLMAPFARFMEHSRLTGDFDESSLEFQRKILERSGLGEETYVPEAMHFVPPRPTMAAAREEAEQVMFGALDTLFANANVNPKDIGILVVNCSLFNPTPSLSAMIVNKYKLRGNIRSFNLGGMGCSAGVIAIDLAKDLLQIHRKTCAVVVSTENITQNWYFGNKKSMLIPNCLFRVGGSAVLLSNKSSDRRRAKYKLVHVVRTHRGADDKAFRCVYQEQDPNGKTGVSLSKDLMAIAGEALKANITTLGPLVLPISEQLLFFSTLVVKKLFNNKVKPYIPDFKLAFEHFCIHAGGRAVIDELEKNLQLSLVHVEASRMTLHRFGNTSSSSIWYELGYIEAKGRMRKGNRVWQIAFGSGFKCNSAVWQALRNVNPSPNGPWEDCIDKYPVKIMS

SEQ ID NO: 23 - DNA, 1512 bp, Linum usitatissimum
  CDS (1)..(1512)
atggactccactgccggcgagattcgaatccaacaaccccgccgcctacccgacttcctgcagagcgtaaacctcaaatacgtcaagctcggctaccactacctcatctccaatctacctaccttattcttcgtccctctaatcgccttaaccttaatcgaagcctcccagatggatcccgatgacctccgccacctctggctccacctccaatacaacctcgtcgccgtcatcacctgctccgccttcctcgtcttcggtctcaccgtctacatcgtcactcgacctgtccccgtctacctcctcgactacgcctgctaccgccctcccaaaaccctaatggctccttttgcccgcttcatggagcattcccgtctcactggcgacttcgacgagtcttccctcgagtttcagcgcaagatcctcgagcgatccggactcggcgaggagacttacgtccccgaggcgatgcacttcgtcccaccgcggccgacgatggctgccgccagggaggaggctgagcaggtaatgttcggtgccttggatactctattcgccaatgccaatgtaaaccctaaggatattgggattttggtagtcaattgtagcctgttcaatcccactccttccctctcggcgatgattgttaataagtacaagctccgtgggaatatccggagctttaatttgggtggtatggggtgtagcgccggtgtaattgcaatcgatttggccaaggatcttcttcaaatccaccggaaaacttgcgctgtcgtcgtgagtactgagaacattactcagaactggtactttgggaacaaaaaatcgatgctgatccctaattgcttgttcagagtgggtggatcggcagtcctgctctcgaataaatcgagtgataggaggagagccaagtacaagctcgtccatgtcgtacggactcacagaggagcagacgacaaggcttttcgctgcgtttaccaggagcaggatcccaatgggaagactggggtttcattgtcaaaggatttgatggcgattgcaggggaggcattgaaggcaaacattacgactctgggtcctctggtccttcctattagtgagcagcttctcttcttctcgacattggtcgtgaagaagctgttcaacaacaaggtgaagccgtatatcccagatttcaagctcgcattcgaacatttctgtatccacgcaggcgggagggctgtgatcgatgagctggagaagaatttgcagctgtctcttgttcatgtggaagcatcaaggatgactcttcataggtttggcaacacctcgtcgagttcaatctggtacgaattgggctacattgaggcgaaagggaggatgaggaaagggaatcgggtgtggcagattgcgtttgggagtgggtttaagtgtaacagcgctgtgtggcaggcgctgaggaatgtgaatccctcgccaaacggaccgtgggaagattgcatagataagtacccagttaagataatgtcttga

SEQ ID NO: 24 - protein, 503 aa, Linum usitatissimum; identical to SEQ ID NO: 22

(19) United States
(12) Patent Application Publication
(10) Pub. No.: US 2008/0120748 A1
(43) Pub. Date: May 22, 2008

(54) Nucleic Acid Molecules Encoding Kcs-Like Polypeptides And Methods Of Use

(75) Inventors: Thorsten Zank, Mannheim (DE); Oliver Oswald, Ludwigshafen (DE)

Correspondence Address: CONNOLLY BOVE LODGE & HUTZ, LLP, P O BOX 2207, WILMINGTON, DE, 19899, US

(73) Assignee: BASF Plant Science GmbH, Ludwigshafen (DE)

(21) Appl. No.: 11/793,630

(22) PCT Filed: Dec. 20, 2005
(86) PCT No.: PCT/EP05/13686

§ 371 Date: Jul. 12, 2007

Related U.S. Application Data
(60) Provisional application No. 60/637,631, filed on Dec. 20, 2004.

(51) Int. Cl. C12N 15/82; C12N 9/00; C12N 15/11; A01H 5/00; C12N 15/00

(52) U.S. Cl. 800/281; 435/183; 536/23.2; 435/320.1; 800/278; 800/298; 800/320; 800/320.1; 800/320.2; 800/320.3; 800/312; 800/314; 800/322; 800/317.1

(57) ABSTRACT

This invention relates generally to nucleic acid sequences encoding proteins that are related to the presence of seed storage compounds in plants. More specifically, the present invention relates to KCS-like nucleic acid sequences encoding lipid metabolism proteins (LMP) and the use of these sequences in transgenic plants. In particular, the invention is directed to methods for manipulating fatty acid-related compounds and for increasing oil level and altering the fatty acid composition in plants and seeds. The invention further relates to methods of using these novel plant polypeptides to stimulate plant growth and/or to increase yield and/or composition of seed storage compounds.

### FIELD OF THE INVENTION

Described herein are inventions in the field of genetic engineering of plants, including isolated nucleic acid molecules encoding putative fatty acid elongase beta-ketoacyl-CoA synthase (KCS) to improve agronomic, horticultural and quality traits. This invention relates generally to nucleic acid sequences encoding proteins that are related to the presence of seed storage compounds in plants. More specifically, the present invention relates to KCS-like nucleic acid sequences encoding lipid metabolism proteins (LMP) and the use of these sequences in transgenic plants. In particular, the invention is directed to methods for manipulating fatty acid-related compounds and for increasing oil level and altering the fatty acid composition in plants and seeds. The invention further relates to methods of using these novel plant polypeptides to stimulate plant growth and/or to increase yield and/or composition of seed storage compounds.

### BACKGROUND OF THE INVENTION

The study and genetic manipulation of plants has a long history that began even before the framed studies of Gregor Mendel. In perfecting this science, scientists have accomplished modification of particular traits in plants ranging from potato tubers having increased starch content to oilseed plants such as canola and sunflower having increased or altered fatty acid content. With the increased consumption and use of plant oils, the modification of seed oil content and seed oil levels has become increasingly widespread (e.g. Töpfer et al. 1995, Science 268:681-686). Manipulation of biosynthetic pathways in transgenic plants provides a number of opportunities for molecular biologists and plant biochemists to affect plant metabolism giving rise to the production of specific higher-value products. The seed oil production or composition has been altered in numerous traditional oilseed plants such as soybean (U.S. Pat. No. 5,955,650), canola (U.S. Pat. No. 5,955,650), sunflower (U.S. Pat. No. 6,084,164) and rapeseed (Töpfer et al. 1995, Science 268:681-686), and non-traditional oil seed plants such as tobacco (Cahoon et al. 1992, Proc. Nat. Acad. Sci. USA 89:11184-11188).

Plant seed oils comprise both neutral and polar lipids (see Table 1). The neutral lipids contain primarily triacylglycerol, which is the main storage lipid that accumulates in oil bodies in seeds. The polar lipids are mainly found in the various membranes of the seed cells, e.g. the endoplasmic reticulum, microsomal membranes, plastidial and mitochondrial membranes and the cell membrane. The neutral and polar lipids contain several common fatty acids (see Table 2) and a range of less common fatty acids. The fatty acid composition of membrane lipids is highly regulated and only a select number of fatty acids are found in membrane lipids. On the other hand, a large number of unusual fatty acids can be incorporated into the neutral storage lipids in seeds of many plant species (Van de Loo F. J. et al. 1993, Unusual Fatty Acids in Lipid Metabolism in Plants pp. 91-126, editor T S Moore Jr. CRC Press; Millar et al. 2000, Trends Plant Sci. 5:95-101).

Lipids are synthesized from fatty acids and their synthesis may be divided into two parts: the prokaryotic pathway and the eukaryotic pathway (Browse et al. 1986, Biochemical J. 235:25-31; Ohlrogge & Browse 1995, Plant Cell 7:957-970). The prokaryotic pathway is located in plastids that are also the primary site of fatty acid biosynthesis. Fatty acid synthesis begins with the conversion of acetyl-CoA to malonyl-CoA by acetyl-CoA carboxylase (ACCase). Malonyl-CoA is converted to malonyl-acyl carrier protein (ACP) by the malonyl-CoA:ACP transacylase. The enzyme beta-keto-acyl-ACP-synthase III (KAS III) catalyzes a condensation reaction, in which the acyl group from acetyl-CoA is transferred to malonyl-ACP to form 3-ketobutyryl-ACP. In a subsequent series of condensation, reduction and dehydration reactions the nascent fatty acid chain on the ACP cofactor is elongated by the step-by-step addition (condensation) of two carbon atoms donated by malonyl-ACP until a 16- or 18-carbon saturated fatty acid chain is formed. The plastidial delta-9 acyl-ACP desaturase introduces the first double bond into the fatty acid.

In the prokaryotic pathway the saturated and monounsaturated acylACPs are direct substrates for the plastidial glycerol-3-phosphate acyl-transferase and the lysophosphatidic acid acyltransferase, which catalyze the esterification of glycerol-3-phosphate at the sn-1 and sn-2 position. The resulting phosphatidic acid is the precursor for plastidial lipids in which further desaturation of the acyl-residues can occur.

In the eukaryotic lipid biosynthesis pathway thioesterases cleave the fatty acids from the ACP cofactor and free fatty acids are exported to the cytoplasm where they participate as fatty acyl-CoA esters in the eukaryotic pathway. In this pathway the fatty acids are esterified by glycerol-3-phosphate acyltransferase and lysophosphatidic acid acyl-transferase to the sn-1 and sn-2 positions of glycerol-3-phosphate, respectively, to yield phosphatidic acid (PA). The PA is the precursor for other polar and neutral lipids, the latter being formed in the Kennedy or other pathways (Voelker 1996, Genetic Engineering ed.:Setlow 18:111-113; Shanklin & Cahoon 1998, Annu. Rev. Plant Physiol. Plant Mol. Biol. 49:611-641; Frentzen 1998, Lipids 100:161-166; Millar et al. 2000, Trends Plant Sci. 5:95-101).

The acyl-CoAs resulted from the export of plastidic fatty acids can also be elongated to yield very-long-chain fatty acids with more than 18 carbon atoms. Fatty acid elongases are multienzyme complexes consisting of at least four enzyme activities: beta-ketoacyl-CoA synthases, beta-ketoacyl-CoA reductase, beta-hydroxyacyl-CoA dehydratase and enoyl-CoA reductase. It is well known that the beta-ketoacyl-CoA synthase determines the activity and the substrate selectivity of the fatty acid elongase complex (Millar & Kunst 1997, Plant J. 12:121-131). The very-long-chain fatty acids can be either used for wax and sphingolipid biosynthesis or enter the pathways for seed storage lipid biosynthesis.

Storage lipids in seeds are synthesized from carbohydrate-derived precursors. Plants have a complete glycolytic pathway in the cytosol (Plaxton 1996, Annu. Rev. Plant Physiol. Plant Mol. Biol. 47:185-214) and it has been shown that a complete pathway also exists in the plastids of rapeseeds (Kang & Rawsthorne 1994, Plant J. 6:795-805). Sucrose is the primary source of carbon and energy, transported from the leaves into the developing seeds. During the storage phase of seeds, sucrose is converted in the cytosol to provide the metabolic precursors glucose-6-phosphate and pyruvate. These are transported into the plastids and converted into acetyl-CoA that serves as the primary precursor for the synthesis of fatty acids. Acetyl-CoA in the plastids is the central precursor for lipid biosynthesis. Acetyl-CoA can be formed in the plastids by different reactions and the exact contribution of each reaction is still being debated (Ohlrogge & Browse 1995, Plant Cell 7:957-970). It is however accepted that a large part of the acetyl-CoA is derived from glucose-6-phosphate and pyruvate that are imported from the cytoplasm into the plastids. Sucrose is produced in the source organs (leaves, or anywhere where photosynthesis occurs) and is transported to the developing seeds that are also termed sink organs. In the developing seeds, sucrose is the precursor for all the storage compounds, i.e. starch, lipids and partly the seed storage proteins.

Storage compounds such as triacylglycerols (seed oil) serve as carbon and energy reserves, which are used during germination and growth of the young seedling. Seed (vegetable) oil is also an essential component of the human diet and a valuable commodity providing feedstocks for the chemical industry.

Although the lipid and fatty acid content and/or composition of seed oil can be modified by the traditional methods of plant breeding, the advent of recombinant DNA technology has allowed for easier manipulation of the seed oil content of a plant, and in some cases, has allowed for the alteration of seed oils in ways that could not be accomplished by breeding alone (see, e.g., Töpfer et al., 1995, Science 268:681-686). For example, introduction of a Δ¹²-hydroxylase nucleic acid sequence into transgenic tobacco resulted in the introduction of a novel fatty acid, ricinoleic acid, into the tobacco seed oil (Van de Loo et al. 1995, Proc. Natl. Acad. Sci. USA 92:6743-6747). Tobacco plants have also been engineered to produce low levels of petroselinic acid by the introduction and expression of an acyl-ACP desaturase from coriander (Cahoon et al. 1992, Proc. Natl. Acad. Sci. USA 89:11184-11188).

The modification of seed oil content in plants has significant medical, nutritional and economic ramifications. With regard to the medical ramifications, the long chain fatty acids (C18 and longer) found in many seed oils have been linked to reductions in hypercholesterolemia and other clinical disorders related to coronary heart disease (Brenner 1976, Adv. Exp. Med. Biol. 83:85-101). Therefore, consumption of a plant having increased levels of these types of fatty acids may reduce the risk of heart disease. Enhanced levels of seed oil content also increase large-scale production of seed oils and thereby reduce the cost of these oils.

In order to increase or alter the levels of compounds such as seed oils in plants, nucleic acid sequences and proteins regulating lipid and fatty acid metabolism must be identified. As mentioned earlier, several desaturase nucleic acids such as the Δ⁶-desaturase nucleic acid, Δ¹²-desaturase nucleic acid and acyl-ACP desaturase nucleic acid have been cloned and demonstrated to encode enzymes required for fatty acid synthesis in various plant species. Oleosin nucleic acid sequences from such different species as canola, soybean, carrot, pine and Arabidopsis thaliana have also been cloned and determined to encode proteins associated with the phospholipid monolayer membrane of oil bodies in those plants.

It has also been determined that two phytohormones, gibberellic acid (GA) and absisic acid (ABA), are involved in overall regulatory processes in seed development (e.g. Ritchie & Gilroy, 1998, Plant Physiol. 116:765-776; ArenasHuertero et al., 2000, Genes Dev. 14:2085-2096). Both the GA and ABA pathways are affected by okadaic acid, a protein phosphatase inhibitor (Kuo et al. 1996, Plant Cell. 8:259-269). The regulation of protein phosphorylation by kinases and phosphatases is accepted as a universal mechanism of cellular control (Cohen, 1992, Trends Biochem. Sci. 17:408-413). Likewise, the plant hormones ethylene (e.g. Zhou et al., 1998, Proc. Natl. Acad. Sci. USA 95:10294-10299; Beaudoin et al., 2000, Plant Cell 2000:1103-1115) and auxin (e.g. Colon-Carmona et al., 2000, Plant Physiol. 124:1728-1738) are involved in controlling plant development as well.

Although several compounds are known that generally affect plant and seed development, there is a clear need to specifically identify factors that are more specific for the developmental regulation of storage compound accumulation and to identify genes which have the capacity to confer altered or increased oil production to its host plant and to other plant species. This invention discloses nucleic acid sequences from Brassica napus, Glycine max or Linum usitatissimum. These nucleic acid sequences can be used to alter or increase the levels of seed storage compounds such as proteins, sugars and oils, in plants, including transgenic plants, such as canola, oilseed rape, linseed, soybean, sunflower, maize, oat, rye, barley, wheat, rice, pepper, tagetes, cotton, oil palm, coconut palm, flax, castor and peanut, which are oilseed plants containing high amounts of lipid compounds.

### BRIEF DESCRIPTION OF THE INVENTION

The present invention provides novel isolated nucleic acid and amino acid sequences associated with the metabolism of seed storage compounds in plants, in particular with sequences that are KCS-like.

Another subject of the present invention is an isolated polypeptide comprising an amino acid sequence selected from the group consisting of
a. LFX¹X²X³LX⁴X⁵X⁶X⁷X⁸F, whereas X² is not M,
b. CYX⁹PX¹⁰X¹¹X¹²X¹³X¹⁴X¹⁵X¹⁶X¹⁷X¹⁸X¹⁹X²⁰X²¹X²²X²³X²⁴X²⁵X²⁶X²⁷X²⁸X²⁹X³⁰X³¹X³²X³³X³⁴X³⁵X³⁶FQ, whereas X¹⁷ ist not Y and whereas X³² is not D and whereas X³⁴ ist not A,
c. LKYVKLGYX³⁷YX³⁸X³⁹X⁴⁰X⁴¹X⁴²X⁴³X⁴⁴X⁴⁵X⁴⁶X⁴⁷X⁴⁸P, whereas X⁴⁸ is not F,
d. AFGSGFKCNSX⁴⁹X⁵⁰W, whereas X⁵⁰ is not 1, and
e. X⁵¹X⁵²X⁵³X⁵⁴X⁵⁵FQX⁵⁶X⁵⁷X⁵⁸LX⁵⁹X⁶⁰SGLG, whereas X⁵¹ is not D and whereas X⁵³ is not A
   and whereas X has the meaning of any amino acid if not defined elsewhise above.

A sequence alignment for determining the common peptide sequences a to e of claim 1 is preferably generated using Align X (Aug. 22, 2003) of Vector NTI Suite 9.0. The parameters used for the multiple alignment were as follows: Gap opening penalty: 10; Gap extension penalty: 0.05; Gap separation penalty range: 8; % identity for alignment delay: 40

The isolated polypeptide of the present invention can include one, two, three, four or five of the amino acid sequences of claim 1. X stands for any amino acid if not defined elsewhise in claim 1, especially an amino acid selected from the group consisting of G, A, V, L, I, F, Y, W, P, D, E, N, Q, S, T, C, M, K, R and H.

X² is not M. X² is in a preferred embodiment an amino acid selected from the group consisting of G, A, V, L, I, F, Y, W, P, D, E, N, Q, S, T, C, K, R and H, in a more preferred embodiment from the group consisting of L, S and A.

X¹⁷ ist not Y. X¹⁷ is in a preferred embodiment an amino acid selected from the group consisting of G, A, V, L, I, F, W, P, D, E, N, Q, S, T, C, M, K, R and H, in a more preferred embodiment from the group consisting of V, F, R and K.

X³² is not D. X³² is in a preferred embodiment an amino acid selected from the group consisting of G, A, V, L, I, F, Y, W, P, E, N, Q, S, T, C, M, K, R and H, in a more preferred embodiment from the group consisting of E and P.

X³⁴ ist not A. X³⁴ is in a preferred embodiment an amino acid selected from the group consisting of G, V, L, I, F, Y, W, P, D, E, N, Q, S, T, C, M, K, R and H, in a more preferred embodiment from the group consisting of S and N.

X⁴⁸ is not F. X⁴⁸ is in a preferred embodiment an amino acid selected from the group consisting of G, A, V, L, I, Y, W, P, D, E, N, Q, S, T, C, M, K, R and H, in a more preferred embodiment from the group consisting of L, V, I and S.

X⁵⁰ is not I. X⁵⁰ is in a preferred embodiment an amino acid selected from the group consisting of G, A, V, L, F, Y, W, P, D, E, N, Q, S, T, C, M, K, R and H, in a more preferred embodiment X⁵⁰ is V.

X⁵¹ is not D. X⁵¹ is in a preferred embodiment an amino acid selected from the group consisting of G, A, V, L, I, F, Y, W, P, E, N, Q, S, T, C, M, K, R and H, in a more preferred embodiment from the group consisting of E and P.

X⁵³ is not A. X⁵³ is in a preferred embodiment an amino acid selected from the group consisting of G, V, L, I, F, Y, W, P, D, E, N, Q, S, T, C, M, K, R and H, in a more preferred embodiment from the group consisting of S and N.

In another embodiment of the present invention the above mentioned isolated polypeptide (of claim 1) functions as a modulator of a seed storage compound in microorganisms or in plants.

In another embodiment of the present invention the above mentioned isolated polypeptide (as defined in claim 1) is used to increase the total seed oil content in the transgenic plant as compared to an the wild type variety of the plant, preferably by 1 weight-%, 2.5 weight-%, 5 weight-%, 7.5 weight-%, 10 weight-%, 12.5 weight-%, 15 weight-%, 17.5 weight-%, 20 weight-%, 22.5 weight-% or 25 weight-% or more.

In a preferred embodiment the above mentioned isolated polypeptide (as defined in claim 1) has a polypeptide sequence as depicted in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22 or SEQ ID NO: 24.

In a further embodiment the above mentioned isolated polypeptide (as defined in claim 1) is an isolated polypeptide selected from the group consisting of
a. a polypeptide sequence encoded by a polynucleotide sequence as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23;
b. a polypeptide sequence as depicted in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22 or SEQ ID NO: 24; and
c. a polypeptide sequence having at least 70% sequence identity with the polypeptide sequence of a) or b) above.

The present invention provides moreover an isolated nucleic acid comprising a polynucleotide sequence selected from the group consisting of:
a. a polynucleotide sequence as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23;
b. a polynucleotide sequence encoding a polypeptide as depicted in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22 or SEQ ID NO: 24;
c. a polynucleotide sequence having at least 70% sequence identity with the nucleic acid of a) or b) above;
d. a polynucleotide sequence that is complementary to the nucleic acid of a) or b) above; and
e. a polynucleotide sequence that hybridizes under stringent conditions to nucleic acid of a) or b) above.

The present invention provides furthermore an isolated nucleic acid encoding a protein containing an amino acid sequence of the present invention (claim 1 or 2) and an isolated polypeptide encoded by this nucleic acid sequence.

In another embodiment of the present invention the above mentioned isolated nucleic acid functions as a modulator of a seed storage compound in microorganisms or in plants.

In another embodiment of the present invention the above mentioned isolated nucleic acid is used to increase the total seed oil content in the transgenic plant as compared to an the wild type variety of the plant, preferably by 1 weight-%, 2.5 weight-%, 5 weight-%, 7.5 weight-%, 10 weight-%, 12.5 weight-%, 15 weight-%, 17.5 weight-%, 20 weight-%, 22.5 weight-% or 25 weight-% or more.

The present invention provides furthermore isolated polypeptide selected from the group consisting of
a. a polypeptide sequence encoded by a polynucleotide sequence as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23;
b. a polypeptide sequence as depicted in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22 or SEQ ID NO: 24; and
c. a polypeptide sequence having at least 70% sequence identity with the polypeptide sequence of a) or b) above.

The present invention provides in a preferred embodiment an isolated polypeptide that is at least about 70-80%, 80-90%, or 90-95%, that means preferably 70%, 72.5%, 75%, 77.5%, 80%, 82.5%, 85%, 87.5%, 90% or 92.5% or more and even more preferably at least about 95%, 96%, 97%, 98%, 99% or more homologous to a polypeptide sequence as depicted in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22 or SEQ ID NO: 24. Sequence identity is to be understood as sequence homology.

In another embodiment of the present invention the above mentioned isolated polypeptide functions as a modulator of a seed storage compound in microorganisms or in plants.

In another embodiment of the present invention the above mentioned isolated polypeptide is used to increase the total seed oil content in the transgenic plant as compared to the wild type variety of the plant, preferably by 1 weight-%, 2.5 weight-%, 5 weight-%, 7.5 weight-%, 10 weight-%, 12.5 weight-%, 15 weight-%, 17.5 weight-%, 20 weight-%, 22.5 weight-% or 25 weight-% or more.

Arabidopsis plants are known to produce considerable amounts of fatty acids like linoleic and linolenic acid (see, e.g., Table 2) and for their close similarity in many aspects (gene homology etc.) to the oil crop plant Brassica. Therefore, nucleic acid molecules originating from a plant like Brassica napus, Glycine max or Linum usitatissimum or related organisms are especially suited to modify the lipid and fatty acid metabolism in a host, especially in microorganisms and plants. Furthermore, nucleic acids from the plant Brassica napus, Glycine max or Linum usitatissimum or related organisms can be used to identify those DNA sequences and enzymes in other species, which are useful to modify the biosynthesis of precursor molecules of fatty acids in the respective organisms.

Also provided by the present invention are polypeptides encoded by the nucleic acids, and heterologous polypeptides comprising polypeptides encoded by the nucleic acids, and antibodies to those polypeptides.

Additionally, the present invention relates to and provides the use of LMP nucleic acids in the production of transgenic plants having a modified level or composition of a seed storage compound. In regard to an altered composition, the present invention can be used to, for example, increase the percentage of oleic acid relative to other plant oils, by e.g. 1 weight-%, 2.5 weight-%, 5 weight-%, 7.5 weight-%, 10 weight-%, 12.5 weight-%, 15 weight-%, 17.5 weight-%, 20 weight-%, 22.5 weight-% or 25 weight-% or more. A method of producing a transgenic plant with a modified level or composition of a seed storage compound includes the steps of transforming a plant cell with an expression vector comprising a LMP nucleic acid, and generating a plant with a modified level or composition of the seed storage compound from the plant cell. In a preferred embodiment, the plant is an oil producing species selected from the group consisting of canola, linseed, soybean, sunflower, maize, oat, rye, barley, wheat, rice, pepper, tagetes, cotton, oil palm, coconut palm, flax, castor and peanut, for example.

According to the present invention, the compositions and methods described herein can be used to alter the composition of a LMP in a transgenic plant and to increase or decrease the level of a LMP in a transgenic plant comprising increasing or decreasing the expression of a LMP nucleic acid in the plant, by e.g. 1%, 2.5%, 5%, 7.5%, 10%, 12.5%, 15%, 17.5%, 20%, 22.5% or 25% or more. Increased or decreased expression of the LMP nucleic acid can be achieved through transgenic overexpression, cosuppression approaches, antisense approaches and in vivo mutagenesis of the LMP nucleic acid. The present invention can also be used to increase or decrease the level of a lipid in a seed oil, preferably by 1 weight-%, 2.5 weight-%, 5 weight-%, 7.5 weight-%, 10 weight-%, 12.5 weight-%, 15 weight-%, 17.5 weight-%, 20 weight-%, 22.5 weight-% or 25 weight-% or more, to increase or decrease the level of a fatty acid in a seed oil, preferably by 1 weight-%, 2.5 weight-%, 5 weight-%, 7.5 weight-%, 10 weight-%, 12.5 weight-%, 15 weight-%, 17.5 weight-%, 20 weight-%, 22.5 weight-% or 25 weight-% or more or to increase or decrease the level of a starch in a seed or plant, preferably by 1 weight-%, 2.5 weight-%, 5 weight-%, 7.5 weight-%, 10 weight-%, 12.5 weight-%, 15 weight-%, 17.5 weight-%, 20 weight-%, 22.5 weight-% or 25 weight-% or more.

More specifically, the present invention includes and provides a method for increasing total oil content in a seeds, preferably by 1 weight-%, 2.5 weight-%, 5 weight-%, 7.5 weight-%, 10 weight-%, 12.5 weight-%, 15 weight-%, 17.5 weight-%, 20 weight-%, 22.5 weight-% or 25 weight-% or more comprising: transforming a plant with a nucleic acid construct that comprises as operably linked components, a promoter and nucleic acid sequences capable of modulating the level of KCS-like mRNA or KCS-like protein, and growing the plant. Furthermore, the present invention includes and provides a method for increasing the level of oleic acid in a seed comprising: transforming a plant with a nucleic acid construct that comprises as operably linked components, a promoter, a structural nucleic acid sequence capable of increasing the level of oleic acid, and growing the plant.

Also included herein is a seed produced by a transgenic plant trans-formed by a LMP DNA sequence, wherein the seed contains the LMP DNA sequence and wherein the plant is true breeding for a modified level of a seed storage compound. The present invention additionally includes a seed oil produced by the aforementioned seed.

Further provided by the present invention are vectors comprising the nucleic acids, host cells containing the vectors, and descendent plant materials produced by transforming a plant cell with the nucleic acids and/or vectors.

According to the present invention, the compounds, compositions, and methods described herein can be used to increase or decrease the relative percentages of a lipid in a seed oil, increase or decrease the level of a lipid in a seed oil, or to increase or decrease the level of a fatty acid in a seed oil, or to increase or decrease the level of a starch or other carbohydrate in a seed or plant, or to increase or decrease the level of proteins in a seed or plant, preferably by 1 weight-%, 2.5 weight-%, 5 weight-%, 7.5 weight-%, 10 weight-%, 12.5 weight-%, 15 weight-%, 17.5 weight-%, 20 weight-%, 22.5 weight-% or 25 weight-% or more. The manipulations described herein can also be used to improve seed germination and growth of the young seedlings and plants and to enhance plant yield of seed storage compounds, preferably by 1 weight-%, 2.5 weight-%, 5 weight-%, 7.5 weight-%, 10 weight-%, 12.5 weight-%, 15 weight-%, 17.5 weight-%, 20 weight-%, 22.5 weight-% or 25 weight-% or more.

It is further provided a method of producing a higher or lower, preferably by 1 weight-%, 2.5 weight-%, 5 weight-%, 7.5 weight-%, 10 weight-%, 12.5 weight-%, 15 weight-%, 17.5 weight-%, 20 weight-%, 22.5 weight-% or 25 weight-% or more than normal or typical level of storage compound in a transgenic plant expressing a LMP nucleic acid from, Brassica napus, Glycine max or Linum usitatissimum in the transgenic plant, wherein the transgenic plant is Arabidopsis thaliana, Brassica napus, Glycine max, Oryza satlva, Zea mays, Tnticum aestlvum, Helianthus anuus or Beta vulgaris or a species different from Arabidopsis thaliana, Brassica napus, Glycine max, Oryza satilva or Triticum aestivum. Also included herein are compositions and methods of the modification of the efficiency of production of a seed storage compound, preferably by 1 weight-%, 2.5 weight-%, 5 weight-%, 7.5 weight-%, 10 weight-%, 12.5 weight-%, 15 weight-%, 17.5 weight-%, 20 weight-%, 22.5 weight-% or 25 weight-% or more. As used herein, where the phrase Arabidopsis thaliana, Brassica napus, Glycine max, Oryza sativa, Zea mays, Triticum aestivum, Helianthus anuus or Beta vulgaris used, this also means Arabidopsis thaliana and/or Brassica napus and/or Glycine max and/or Oryza saliva and/or Triticum aestivum and/or Zea mays and/or Helianthus anuus and/or Beta vulgaris.

Accordingly, it is an object of the present invention to provide novel isolated LMP nucleic acids and isolated LMP amino acid sequences from, Brassica napus, Glycine max or Linum usitatissimum as well as active fragments, analogs, and orthologs thereof. Those active fragments, analogs, and orthologs can also be from different plant species as one skilled in the art will appreciate that other plant species will also contain those or related nucleic acids.

It is an embodiment of the invention to provide a method of modulating the level of a seed storage compound weight percentage in a plant comprising, modifying the expression of a nucleic acid in the plant, comprising
i. a first step of introduction into a plant cell of an expression vector comprising a nucleic acid, and
ii. a further step of generating from the plant cell the transgenic plant, wherein the nucleic acid encodes a polypeptide that functions as a modulator of a seed storage compound in the plant wherein the nucleic acid comprises a polynucleotide sequence selected from the group consisting of:
   a. a polynucleotide sequence as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23;
   b. a polynucleotide sequence encoding a polypeptide as depicted in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22 or SEQ ID NO: 24;
   c. a polynucleotide sequence having at least 70% sequence identity with the nucleic acid of a) or b) above;
   d. a polynucleotide sequence that is complementary to the nucleic acid of a) or b) above; and
   e. a polynucleotide sequence that hybridizes under stringent conditions to nucleic acid of a) or b) above.
   The abovementioned inventive method, wherein the total seed oil content weight percentage is increased in the transgenic plant as compared to the wild type variety of the plant.

It is further an embodiment of the invention to provide a method of producing a transgenic plant having a modified level of a seed storage compound weight percentage compared to the wildtype comprising,
i. a first step of introduction into a plant cell of an expression vector containing a nucleic acid, and
ii. a further step of generating from the plant cell the transgenic plant, wherein the nucleic acid encodes a polypeptide that functions as a modulator of a seed storage compound in the plant, and wherein the nucleic acid comprises a polynucleotide sequence selected from the group consisting of:
   a. a polynucleotide sequence as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23;
   b. a polynucleotide sequence encoding a polypeptide as depicted in SEQ ID NO: 2, SEQ ID NO: 4, SEQ ID NO: 6, SEQ ID NO: 8, SEQ ID NO: 10, SEQ ID NO: 12, SEQ. ID NO: 14, SEQ ID NO: 16, SEQ ID NO: 18, SEQ ID NO: 20, SEQ ID NO: 22 or SEQ ID NO: 24;
   c. a polynucleotide sequence having at least 70% sequence identity with the nucleic acid of a) or b) above;
   d. a polynucleotide sequence that is complementary to the nucleic acid of a) or b) above; and
   e. a polynucleotide sequence that hybridizes under stringent conditions to nucleic acid of a) or b) above.
      The abovementioned inventive method, wherein the nucleic acid comprises a polynucleotide sequence having at least 90% sequence identity with the polynucleotide sequence of a) or b) and/or wherein the total seed oil content weight percentage is increased in the transgenic plant as compared to the wild type variety of the plant.

It is another object of the present invention to provide transgenic plants having modified levels of seed storage compounds, and in particular, modified levels of a lipid, a fatty acid or a sugar.

The polynucleotides and polypeptides of the present invention, including agonists and/or fragments thereof, have also uses that include modulating plant growth, and potentially plant yield, preferably increasing plant growth under adverse conditions (drought, cold, light, UV). In addition, antagonists of the present invention may have uses that include modulating plant growth and/or yield, through preferably increasing plant growth and yield. In yet another embodiment, overexpression polypeptides of the present invention using a constitutive promoter may be useful for increasing plant yield under stress conditions (drought, light, cold, UV) by modulating light utilization efficiency. Moreover, polynucleotides and polypeptides of the present invention will improve seed germination and seed dormancy and, hence, will improve plant growth and/or yield of seed storage compounds, preferably by 1 weight-%, 2.5 weight-%, 5 weight-%, 7.5 weight-%, 10 weight-%, 12.5 weight-%, 15 weight-%, 17.5 weight-%, 20 weight-%, 22.5 weight-% or 25 weight-% or more.

The isolated nucleic acid molecules of the present invention may further comprise an operably linked promoter or partial promoter region. The promoter can be a constitutive promoter, an inducible promoter or a tissue-specific promoter. The constitutive promoter can be, for example, the superpromoter (Ni et al., Plant J. 7:661-676, 1995; U.S. Pat. No. 5,955,646) or the PtxA promoter (PF 55368-2 US, Song H. et al., 2004, see Example 11). The tissue-specific promoter can be active in vegetative tissue or reproductive tissue. The tissue-specific promoter active in reproductive tissue can be a seed-specific promoter. The seed-specific promoter can be, for example, the USP promoter (Bäumlein et al. 1991, Mol. Gen. Genetics 225:459-67). The tissue-specific promoter active in vegetative tissue can be a root-specific, shoot-specific, meristem-specific or leaf-specific promoter. The isolated nucleic acid molecule of the present invention can still further comprise a 5′ non-translated sequence, 3′ non-translated sequence, introns, or the combination thereof.

The present invention also provides a method for increasing the number and/or size of one or more plant organs of a plant expressing an isolated nucleic acid from Brassica napus, Glycine max or Linum usitatissimum encoding a Lipid Metabolism Protein (LMP), or a portion thereof. More specifically, seed size and/or seed number and/or weight might be manipulated. Moreover, root length can be increased, by e.g. 1%, 2.5%, 5%, 7.5%, 10%, 12.5%, 15%, 17.5%, 20%, 22.5% or 25% or more. Longer roots can alleviate not only the effects of water depletion from soil but also improve plant anchorage/standability thus reducing lodging. Also, longer roots have the ability to cover a larger volume of soil and improve nutrient uptake. All of these advantages of altered root architecture have the potential to increase crop yield, by e.g. 1%, 2.5%, 5%, 7.5%, 10%, 12.5%, 15%, 17.5%, 20%, 22.5% or 25% or more. Additionally, the number and size of leaves might be increased by the nucleic acid sequences provided in this application. This will have the advantage of improving photosynthetic light utilization efficiency by increasing photosynthetic light capture capacity and photosynthetic efficiency, by e.g. 1%, 2.5%, 5%, 7.5%, 10%, 12.5%, 15%, 17.5%, 20%, 22.5% or 25% or more.

It is a further object of the present invention to provide methods for producing such aforementioned transgenic plants.

It is another object of the present invention to provide seeds and seed oils from such aforementioned transgenic plants.

These and other objects, features and advantages of the present invention will become apparent after a review of the following detailed description of the disclosed embodiments and the appended claims.

### BRIEF DESCRIPTION OF THE DRAWINGS

FIG. 1A-C. Seq ID 1-3—Nucleic acid sequence, open reading frame of the nucleic acid and amino acid sequence of the Brassica napus gene Bn46783084.

FIG. 2A-C. Seq ID 4-6—Nucleic acid sequence, open reading frame of the nucleic acid and amino acid sequence of the Brassica napus gene Bn 44226437.

FIG. 3A-C. Seq ID 7-9—Nucleic acid sequence, open reading frame of the nucleic acid and amino acid sequence of the Glycine max gene Gm59536584.

FIG. 4A-C. Seq ID 10-12—Nucleic acid sequence, open reading frame of the nucleic acid and amino acid sequence of the Glycine max gene Gm59649734.

FIG. 5A-C. Seq ID 13-15—Nucleic acid sequence, open reading frame of the nucleic acid and amino acid sequence of the Glycine max gene Gm 59562371.

FIG. 6A-C. Seq ID 16-18 Nucleic acid sequence, open reading frame of the nucleic acid and amino acid sequence of the Linum usitatissimum gene Lu61779639.

FIG. 07. Schematic of the binary vector that can be used to trans-form the KCS-like genes into Arabidopsis thaliana or crop plants. LB, left border; USP-1, Arabidopsis USP promoter; OCS, octopine synthase termination signal; nosT; nos terminator; CDS(NptII)_{—}5; eukaryotic selection marker; nosp, nos promoter; Bn46783084, cDNA of Bn46783084; RB, right border.

FIG. 08. Total seed oil content in KCS (Atlgl9440) overexpressors of Arabidopsis thaliana in T2 and T3 seed generation. Each circles represents the value obtained with one individual plant. Independent transgenic events are shown. Statistics is t-Test. Col-0, Columbia 0.

FIG. 09. Total seed oil content in segregating Arabidopsis thaliana T2 seeds, overexpressing KCS-orthologous genes from Linum usitatissimum (61779639) or Glycine max (59562371). Each circle represents the average oil value of independent GC measurements of three seed aliquots obtained from one individual plant.

FIG. 10. Fatty acid profile of segregating Arabidopsis thaliana T2 seeds, overexpressing KCS-orthologous genes from Linum usitatissimum (61779639) or Glycine max (59562371). Each bar represents the average values of independent GC measurements of three seed aliquots obtained from one individual plant.

FIG. 11. Diagram illustrating the relative homology among the disclosed Ara KCS-like, Bn44226437, Lu61779639, Bn46783084, Gm59536584, Gm59562371, Gm59649734 amino acid sequences. The diagram was generated using Align X (Aug. 22, 2003) of Vector NTI Suite 9.0. The parameters used for the multiple alignment were as follows: Gap opening penalty: 10; Gap extension penalty: 0.05; Gap separation penalty range: 8; % identity for alignment delay: 40

FIG. 12. Table illustrating the similarity among the Ara KCS-like, Bn44226437, Lu61779639, Bn46783084, Gm59536584, Gm59562371, Gm59649734 amino acid sequences. The table was generated using Align X (Aug. 22, 2003) of Vector NTI Suite 9.0. For other parameters see legend of FIG. 11.

FIG. 13. Diagram illustrating the relative homology among the disclosed Ara KCS-like, Bn44226437, Lu61779639, Bn46783084, Gm59536584, Gm59562371, Gm59649734 nucleic acid sequences. The diagram was generated using Align X (Aug. 22, 2003) of Vector NTI Suite 9.0. The parameters used for the multiple alignment were as follows: Gap opening penalty: 15; Gap extension penalty: 6.66; Gap separation penalty range: 8; % identity for alignment delay: 40

FIG. 14. Table illustrating the similarity among the Ara KCS-like, Bn44226437, Lu61779639, Bn46783084, Gm59536584, Gm59562371, Gm59649734 nucleic acid sequences. The table was generated using Align X (Aug. 22, 2003) of Vector NTI Suite 9.0. For other parameters see legend of FIG. 13.

FIG. 15. Multiple sequence alignment of the Ara KCS-like, Bn44226437, Lu61779639, Bn46783084, Gm59536584, Gm59562371, Gm59649734 amino acid sequences the parameters for the alignment on a protein level are as follows: The alignment was generated using Align X (Aug. 22, 2003) of Vector NTI Suite 9.0. The parameters used for the multiple alignment were as follows: Gap opening penalty: 10; Gap extension penalty: 0.05; Gap separation penalty range: 8; % identity for alignment delay: 40

FIG. 16. Multiple sequence alignment of the Ara KCS-like, Bn44226437, Lu61779639, Bn46783084, Gm59536584, Gm59562371, Gm59649734 nucleic acid sequences. The alignement was generated using Align X (Aug. 22, 2003) of Vector NTI Suite 9.0. The alignment was generated using Align X (Aug. 22, 2003) of Vector NTI Suite 9.0. The parameters used for the multiple alignment were as follows: Gap opening penalty: 15; Gap extension penalty: 6.66; Gap separation penalty range: 8; % identity for alignment delay: 40

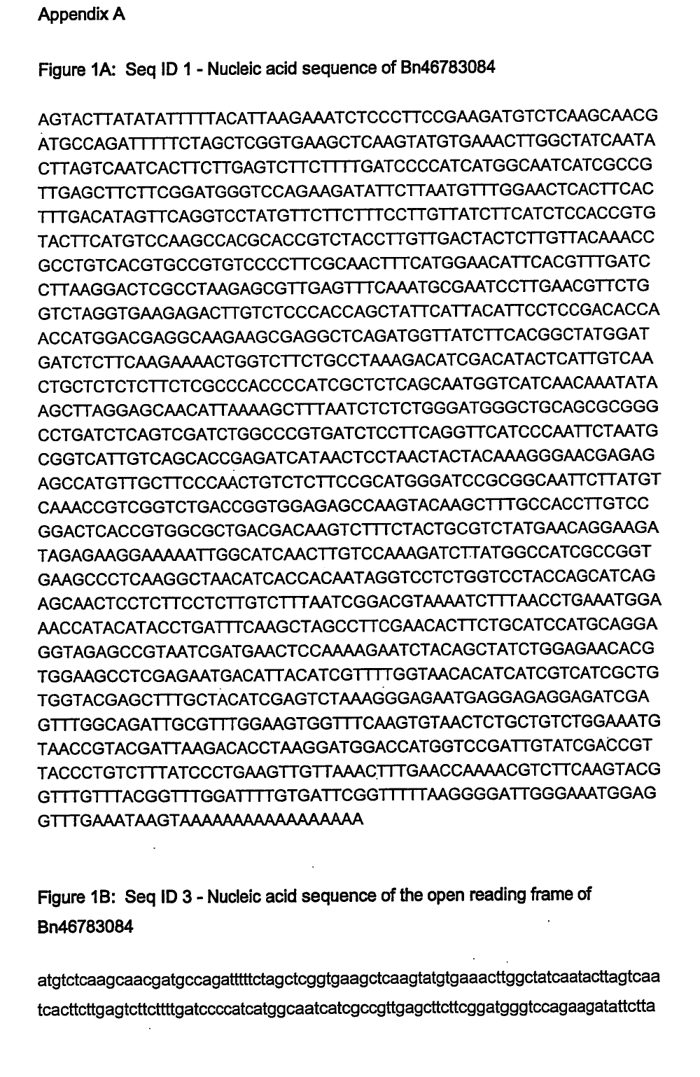

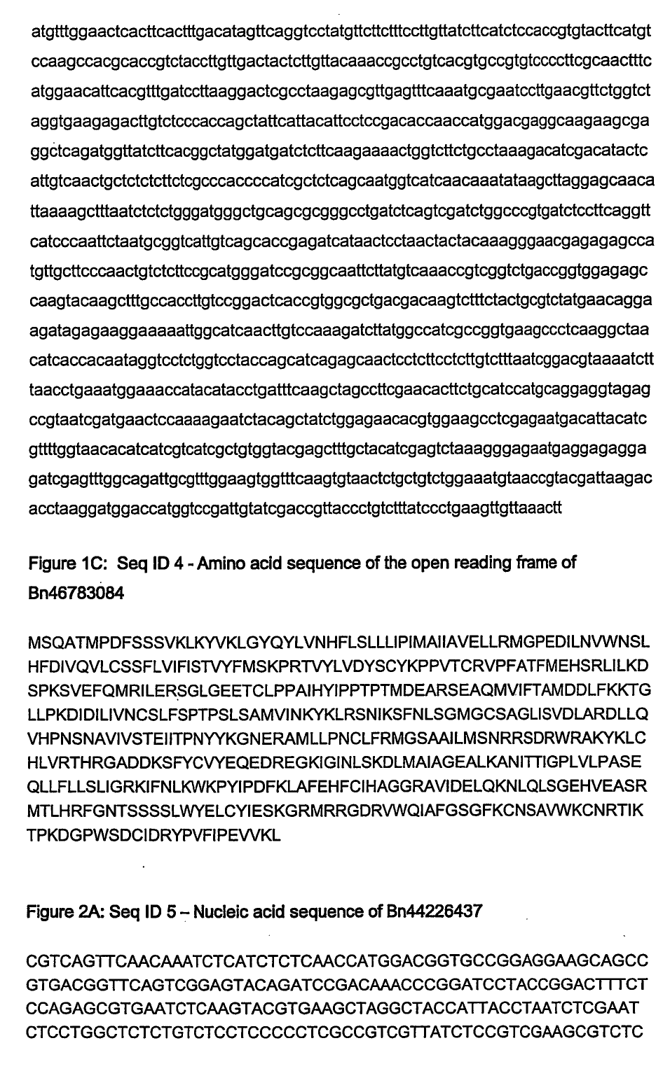

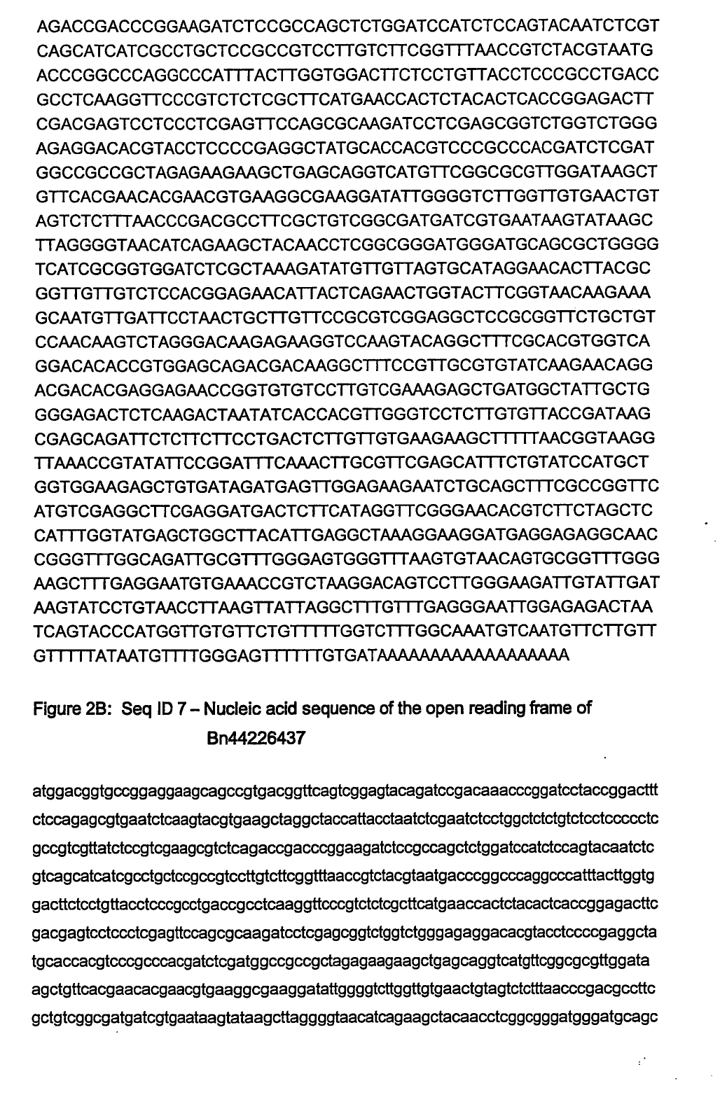

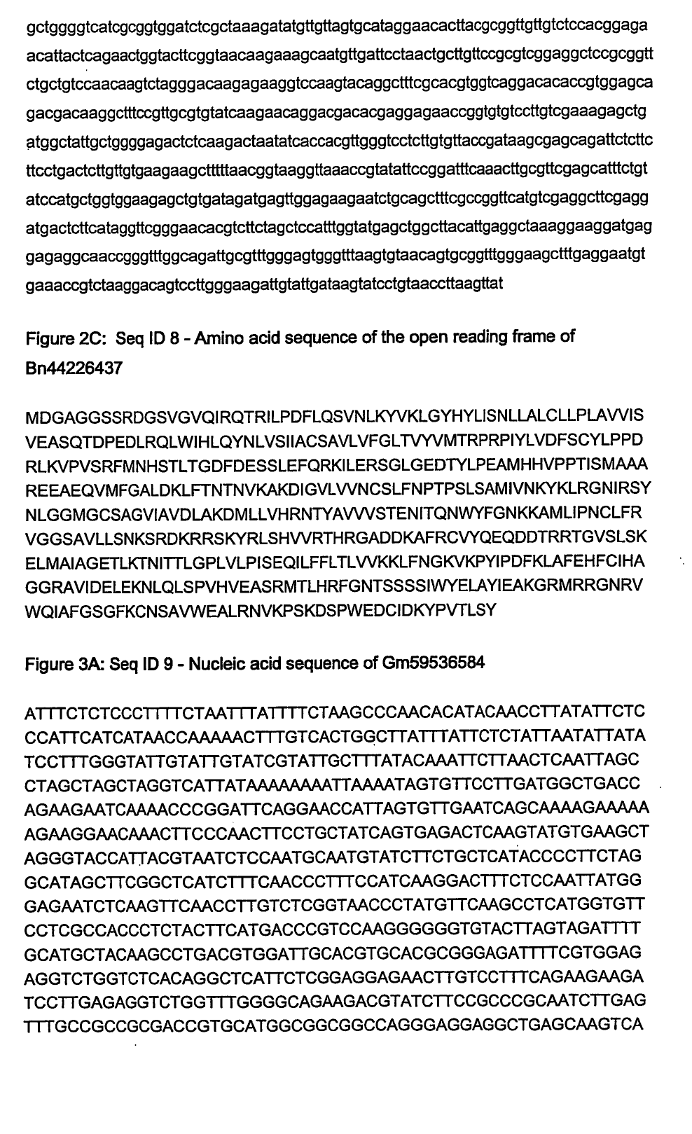

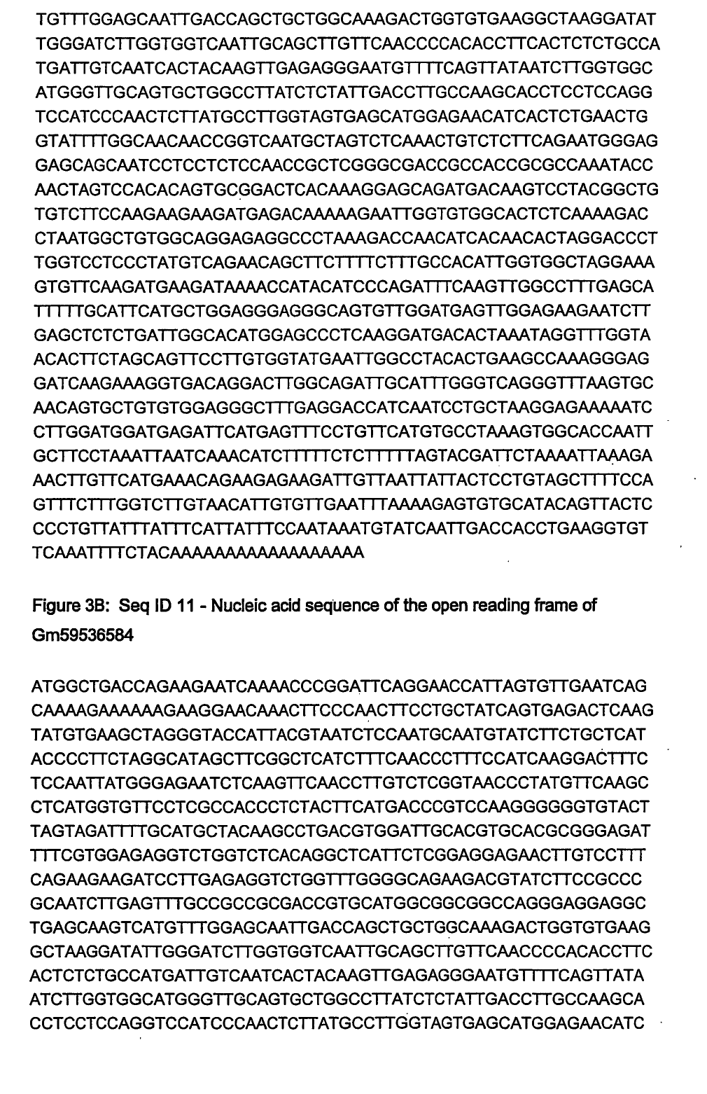

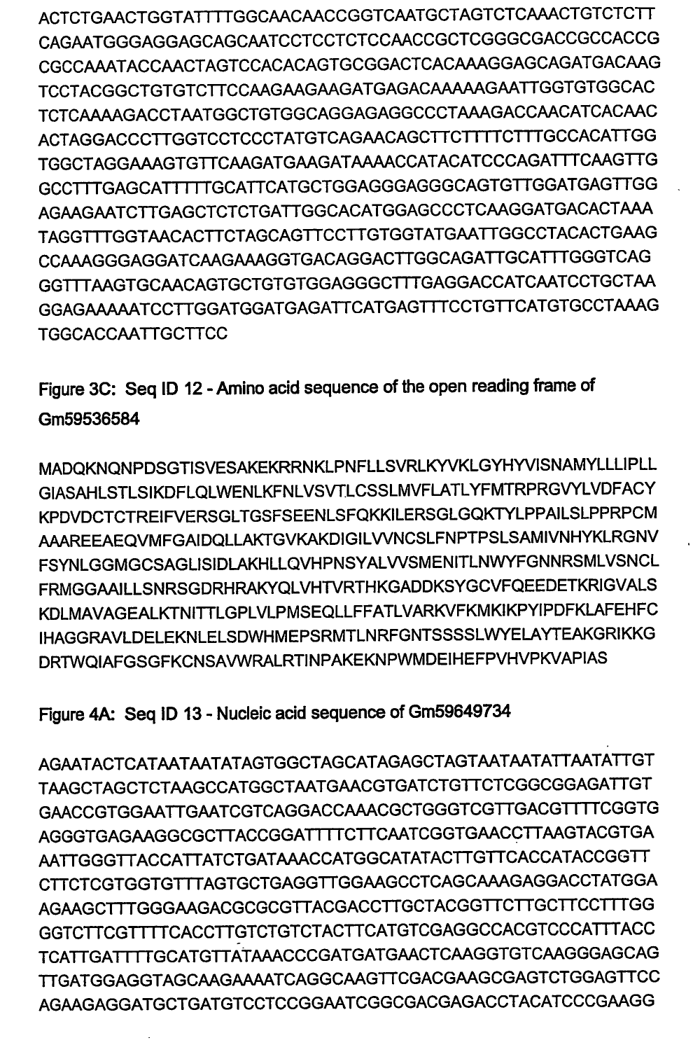

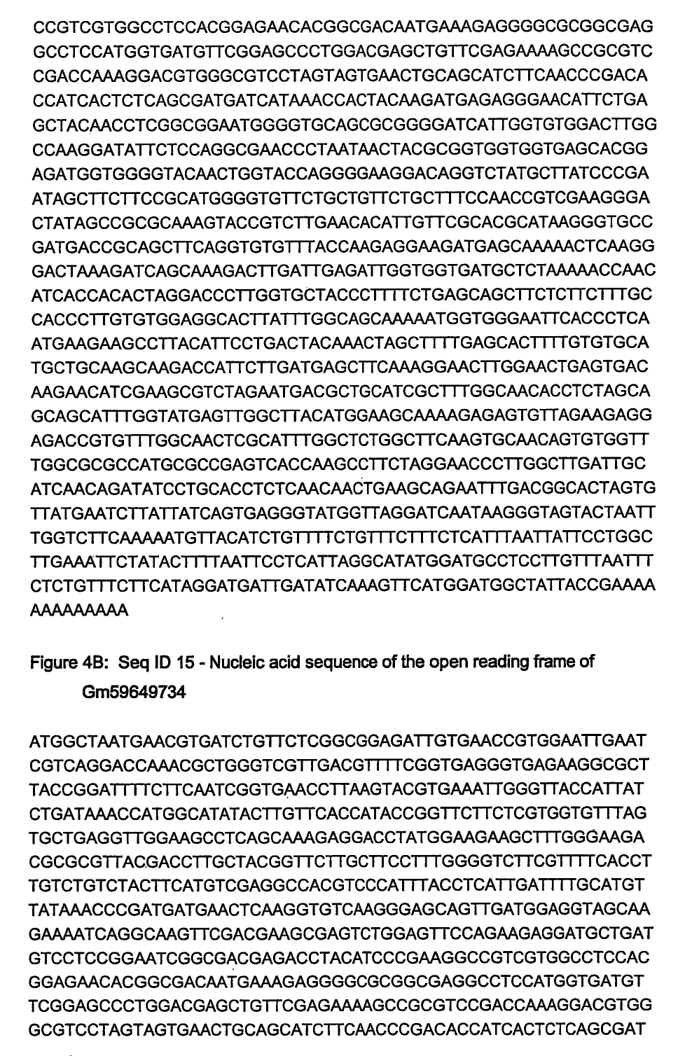

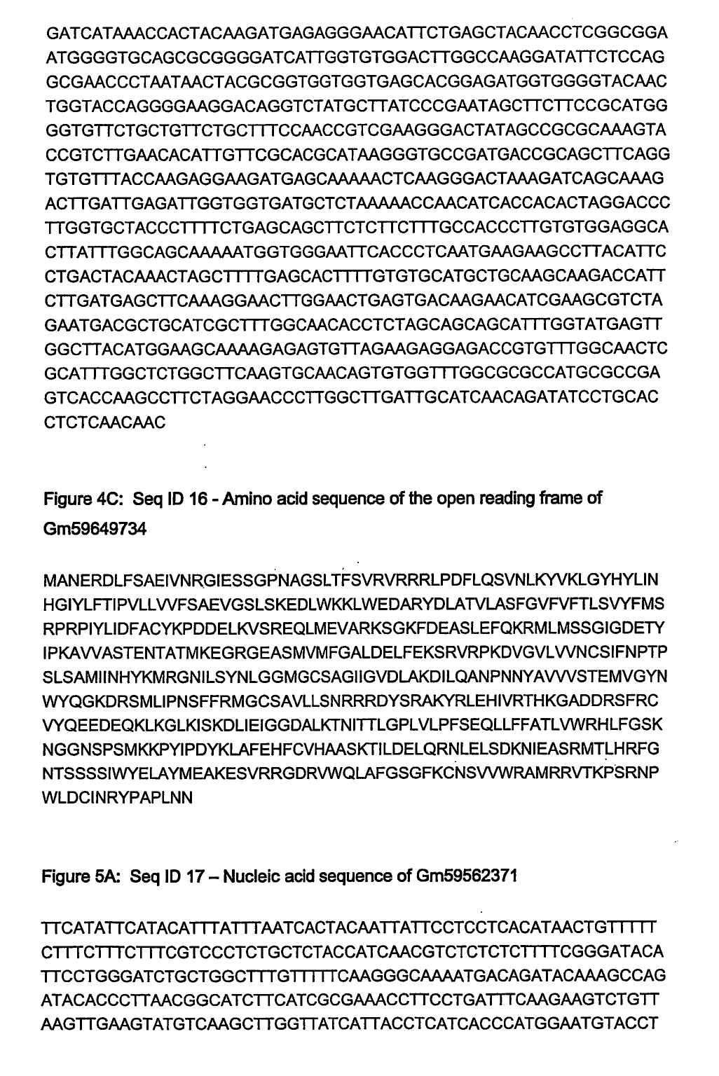

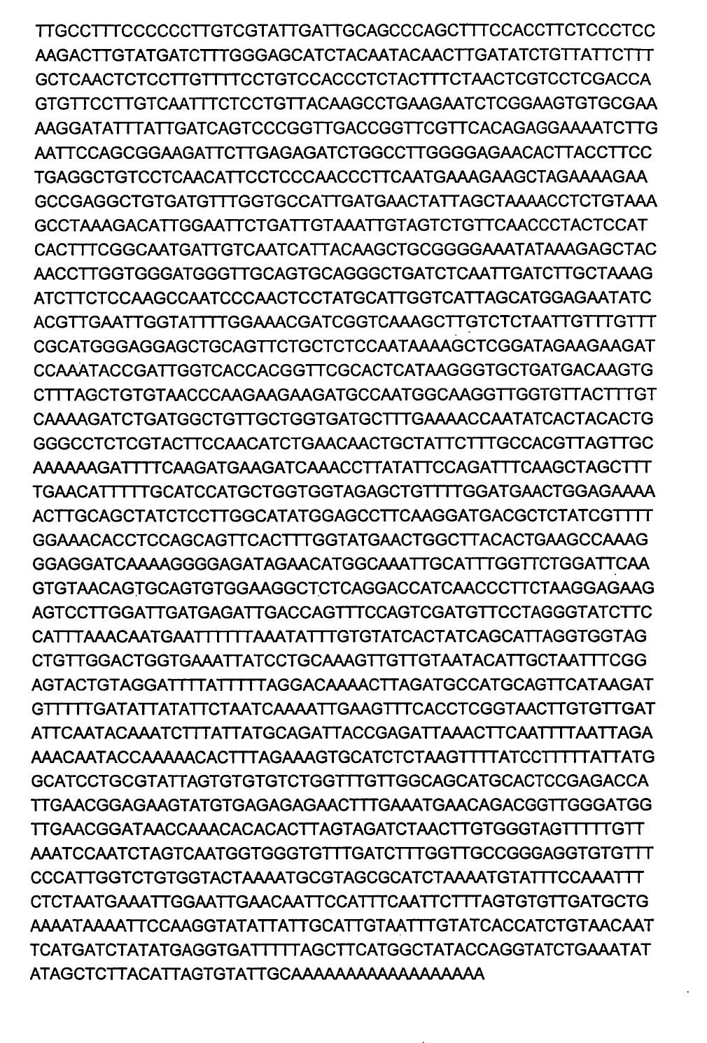

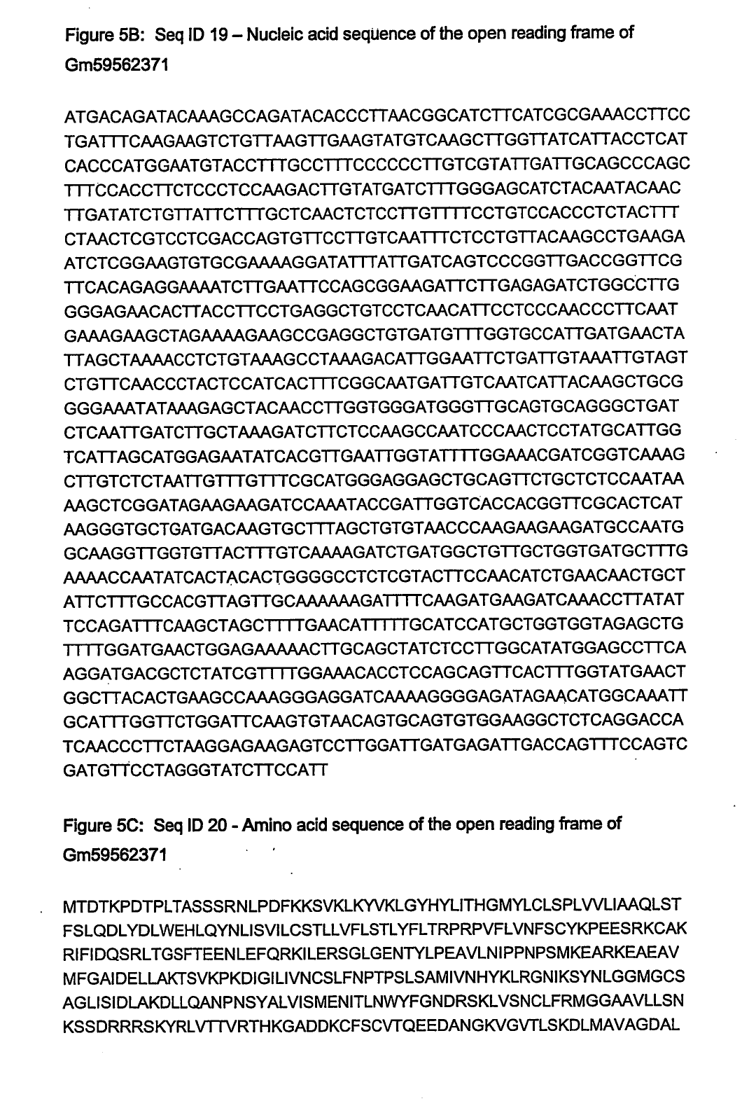

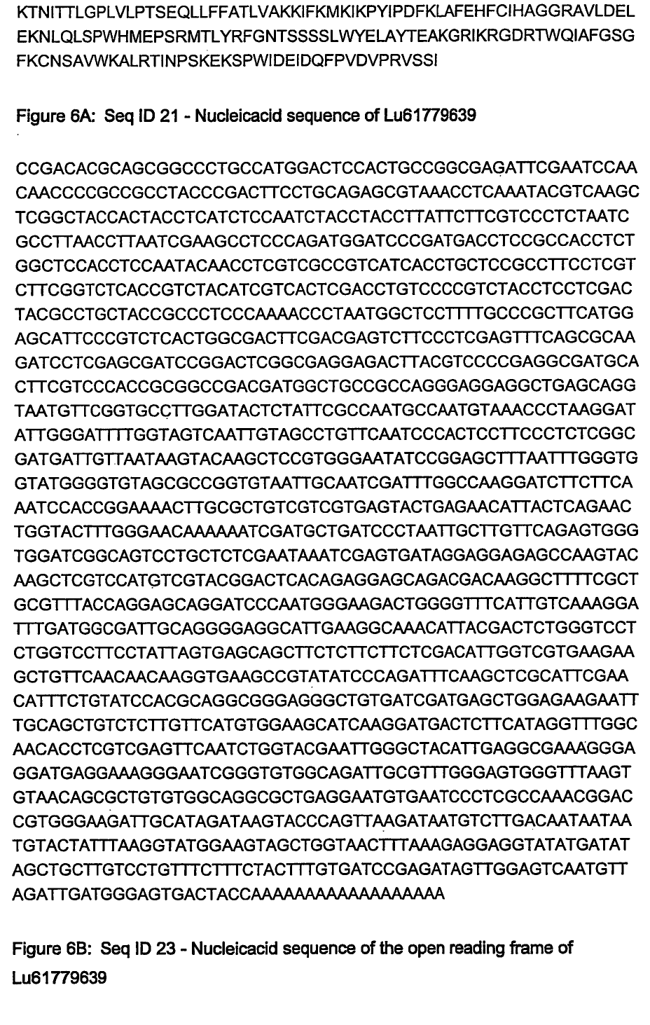

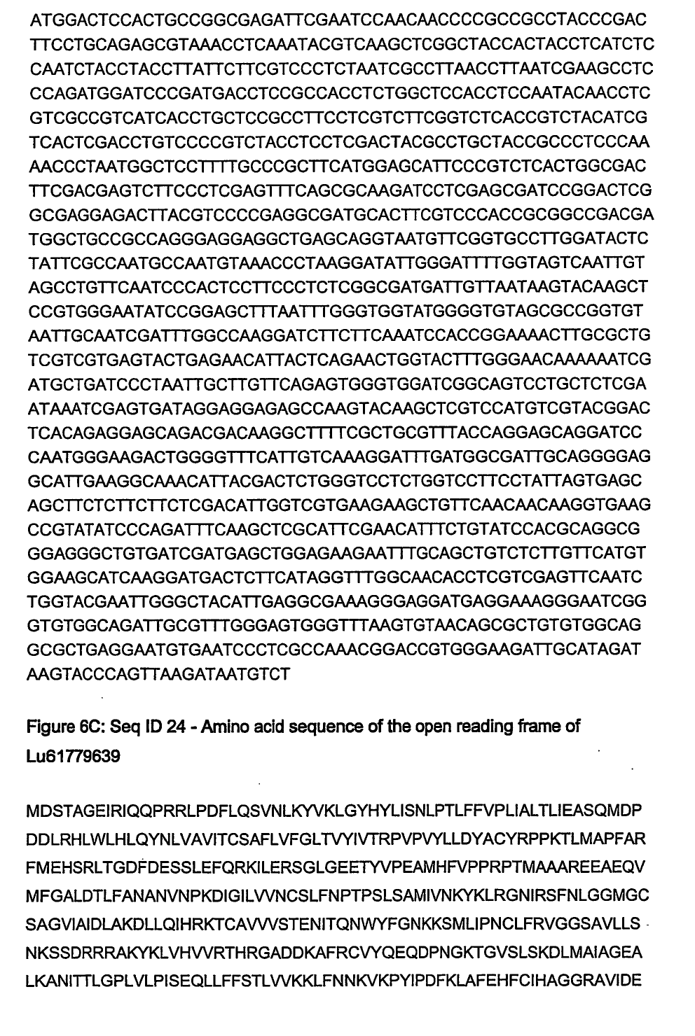

### GENERAL DEFINITIONS

It is to be understood that this invention is not limited to the particular methodology, protocols, cell lines, plant species or genera, constructs, and reagents described as such. It is also to be understood that the terminology used herein is for the purpose of describing particular embodiments only, and is not intended to limit the scope of the present invention, which will be limited only by the appended claims. It must be noted that as used herein and in the appended claims, the singular forms “a,” “and,” and “the” include plural reference unless the context clearly dictates otherwise. Thus, for example, reference to “a vector” is a reference to one or more vectors and includes equivalents thereof known to those skilled in the art, and so forth.

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent, preferably 10 percent, more preferably 5 percent up or down (higher or lower).

As used herein, the word “or” means any one member of a particular list and also includes any combination of members of that list.

As used herein, the term “amino acid sequence” refers to a list of abbreviations, letters, characters or words representing amino acid residues. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. The abbreviations used herein are conventional one letter codes for the amino acids: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine; Z, glutamine or glutamic acid (see L. Stryer, Biochemistry, 1988, W.H. Freeman and Company, New York. The letter “x” as used herein within an amino acid sequence can stand for any amino acid residue.

The term “nucleic acid” refers to deoxyribonucleotides or ribonucleotides and polymers or hybrids thereof in either single- or double-stranded, sense or antisense form.

The phrase “nucleic acid sequence” as used herein refers to a consecubve list of abbreviations, letters, characters or words, which represent nucleotides. In one embodiment, a nucleic acid can be a “probe” which is a relatively short nucleic acid, usually less than 100 nucleotides in length. Often a nucleic acid probe is from about 50 nucleotides in length to about 10 nucleotides in length. A “target region” of a nucleic acid is a portion of a nucleic acid that is identified to be of interest. A “coding region” of a nucleic acid is the portion of the nucleic acid, which is transcribed and translated in a sequence-specific manner to produce into a particular polypeptide or protein when placed under the control of appropriate regulatory sequences. The coding region is said to encode such a polypeptide or protein. Unless otherwise indicated, a particular nucleic acid sequence also implicitly encompasses conservatively modified variants thereof (e.g., degenerate codon substitutions) and complementary sequences, as well as the sequence explicitly indicated. The term “nucleic acid” is used interchangeably herein with “gene”, “cDNA, “mRNA”, “oligonucleotide,” and “polynucleotide”.

As used herein, the terms “complementary” or “complementarity” are used in reference to nucleotide sequences related by the base-pairing rules. For example, the sequence 5′-AGT-3′ is complementary to the sequence 5′-ACT-3′. Complementarity can be “partial” or “total.” “Partial” complementarity is where one or more nucleic acid bases is not matched according to the base pairing rules. “Total” or “complete” complementarity between nucleic acids is where each and every nucleic acid base is matched with another base under the base pairing rules. The degree of complementarity between nucleic acid strands has significant effects on the efficiency and strength of hybridization between nucleic acid strands. A “complement” of a nucleic acid sequence as used herein refers to a nucleotide sequence whose nucleic acids show total complementarity to the nucleic acids of the nucleic acid sequence.

The term “genome” or “genomic DNA” is referring to the heritable genetic information of a host organism. Said genomic DNA comprises the DNA of the nucleus (also referred to as chromosomal DNA) but also the DNA of the plastids (e.g., chloroplasts) and other cellular organelles (e.g., mitochondria). Preferably the terms genome or genomic DNA is referring to the chromosomal DNA of the nucleus.

The term “chromosomal DNA” or “chromosomal DNA-sequence” is to be understood as the genomic DNA of the cellular nucleus independent from the cell cycle status. Chromosomal DNA might therefore be organized in chromosomes or chromatids, they might be condensed or uncoiled. An insertion into the chromosomal DNA can be demonstrated and analyzed by various methods known in the art like e.g., polymerase chain reaction (PCR) analysis, Southern blot analysis, fluorescence in situ hybridization (FISH), and in situ PCR.

The term “wild-type”, “natural” or of “natural origin” means with respect to an organism, polypeptide, or nucleic acid sequence, that said organism is naturally occurring or available in at least one naturally occurring organism which is not changed, mutated, or otherwise manipulated by man.

The terms “heterologous nucleic acid sequence” or “heterologous DNA” are used interchangeably to refer to a nucleotide sequence, which is ligated to, or is manipulated to become ligated to, a nucleic acid sequence to which it is not ligated in nature, or to which it is ligated at a different location in nature. Heterologous DNA is not endogenous to the cell into which it is introduced, but has been obtained from another cell. Generally, although not necessarily, such heterologous DNA encodes RNA and proteins that are not normally produced by the cell into which it is expressed. A promoter, transcription regulating sequence or other genetic element is considered to be “heterologous” in relation to another sequence (e.g., encoding a marker sequence or am agronomically relevant trait) if said two sequences are not combined or differently operably linked their natural environment. Preferably, said sequences are not operably linked in their natural environment (i.e. come from different genes). Most preferably, said regulatory sequence is covalently joined and adjacent to a nucleic acid to which it is not adjacent in its natural environment.

The term “transgene” as used herein refers to any nucleic acid sequence, which is introduced into the genome of a cell or which has been manipulated by experimental manipulations by man. Preferably, said sequence is resulting in a genome which is different from a naturally occurring organism (e.g., said sequence, if endogenous to said organism, is introduced into a location different from its natural location, or its copy number is increased or decreased). A transgene may be an “endogenous DNA sequence”, “an “exogenous DNA sequence” (e.g., a foreign gene), or a “heterologous DNA sequence”. The term “endogenous DNA sequence” refers to a nucleotide sequence, which is naturally found in the cell into which it is introduced so long as it does not contain some modification (e.g., a point mutation, the presence of a selectable marker gene, etc.) relative to the naturally-occurring sequence.

The term “transgenic” or “recombinant” when used in reference to a cell or an organism (e.g., with regard to a barley plant or plant cell) refers to a cell or organism which contains a transgene, or whose genome has been altered by the introduction of a transgene. A transgenic organism or tissue may comprise one or more transgenic cells. Preferably, the organism or tissue is substantially consisting of transgenic cells (i.e., more than 80%, preferably 90%, more preferably 95%, most preferably 99% of the cells in said organism or tissue are transgenic).

A “recombinant polypeptide” is a non-naturally occurring polypeptide that differs in sequence from a naturally occurring polypeptide by at least one amino acid residue. Preferred methods for producing said recombinant polypeptide and/or nucleic acid may comprise directed or non-directed mutagenesis, DNA shuffling or other methods of recursive recombination.

The term “equivalent” when made in reference to a hybridization condition as it relates to a hybridization condition of interest means that the hybridizabon condition and the hybridization condition of interest result in hybridization of nucleic acid sequences which have the same range of percent (%) homology. For example, if a hybridization condition of interest results in hybridization of a first nucleic acid sequence with other nucleic acid sequences that have from 80% to 90% homology to the first nucleic acid sequence, then another hybridization condition is said to be equivalent to the hybridization condition of interest if this other hybridization condition also results in hybridization of the first nucleic acid sequence with the other nucleic acid sequences that have from 80% to 90% homology to the first nucleic acid sequence.

In a preferred embodiment for the purposes of the invention, unless defined elsewhise, the percent sequence identity between two nucleic acid or polypeptide sequences is determined using the Vector NTI 7.0 (PC) software package (InforMax, 7600 Wisconsin Ave., Bethesda, Md. 20814). A gap-opening penalty of 15 and a gap extension penalty of 6.66 are used for determining the percent identity of two nucleic acids. A gap-opening penalty of 10 and a gap extension penalty of 0.1 are used for determining the percent identity of two polypeptides. All other parameters are set at the default settings. In a preferred embodiment for the purposes of the invention, unless defined elsewhise, for purposes of a multiple alignment (Clustal W algorithm), the gap-opening penalty is 10, and the gap extension penalty is 0.05 with blosum62 matrix. It is to be understood that for the purposes of determining sequence identity when comparing a DNA sequence to an RNA sequence, a thymidine nucleotide sequence is equivalent to an uracil nucleotide.

When used in reference to nucleic acid hybridization the art knows well that numerous equivalent conditions may be employed to comprise either low or high stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of either low or high stringency hybridization different from, but equivalent to, the above-listed conditions. Those skilled in the art know that whereas higher stringencies may be preferred to reduce or eliminate non-specific binding, lower stringencies may be preferred to detect a larger number of nucleic acid sequences having different homologies.

The term “gene” refers to a coding region operably joined to appropriate regulatory sequences capable of regulating the expression of the polypeptide in some manner. A gene includes untranslated regulatory regions of DNA (e.g., promoters, enhancers, repressors, etc.) preceding (upstream) and following (downstream) the coding region (open reading frame, ORF) as well as, where applicable, intervening sequences (i.e., introns) between individual coding regions (i.e., exons). The term “structural gene” as used herein is intended to mean a DNA sequence that is transcribed into mRNA which is then translated into a sequence of amino acids characteristic of a specific polypeptide.

As used herein the term “coding region” when used in reference to a structural gene refers to the nucleotide sequences which encode the amino acids found in the nascent polypeptide as a result of translation of a mRNA molecule. The coding region is bounded, in eukaryotes, on the 5′side by the nucleotide triplet “ATG” which encodes the initiator methionine and on the 3′-side by one of the three triplets which specify stop codons (i.e., TAA, TAG, TGA). In addition to containing introns, genomic forms of a gene may also include sequences located on both the 5′- and 3′-end of the sequences which are present on the RNA transcript. These sequences are referred to as “flanking” sequences or regions (these flanking sequences are located 5′ or 3′ to the non-translated sequences present on the mRNA transcript). The 5′-flanking region may contain regulatory sequences such as promoters and enhancers which control or influence the transcription of the gene. The 3′-flanking region may contain sequences which direct the termination of transcription, posttranscriptional cleavage and polyadenylation.

The terms “polypeptide”, “peptide”, “oligopeptide”, “polypeptide”, “gene product”, “expression product” and “protein” are used interchangeably herein to refer to a polymer or oligomer of consecutive amino acid residues.

The term “isolated” as used herein means that a material has been removed from its original environment. For example, a naturally-occurring polynucleotide or polypeptide present in a living animal is not isolated, but the same polynucleotide or polypeptide, separated from some or all of the coexisting materials in the natural system, is isolated. Such polynucleotides can be part of a vector and/or such polynucleotides or polypeptides could be part of a composition, and would be isolated in that such a vector or composition is not part of its original environment.

The term “genetically-modified organism” or “GMO” refers to any or ganism that comprises transgene DNA. Exemplary organisms include plants, animals and microorganisms.

The term “cell” or “plant cell” as used herein refers to a single cell. The term “cells” refers to a population of cells. The population may be a pure populatiori comprising one cell type. Likewise, the population may comprise more than one cell type. In the present invention, there is no limit on the number of cell types that a cell population may comprise. The cells may be synchronized or not synchronized. A plant cell within the meaning of this invention may be isolated (e.g., in suspension culture) or comprised in a plant tissue, plant organ or plant at any developmental stage.

The term “organ” with respect to a plant (or “plant organ”) means parts of a plant and may include (but shall not limited to) for example roots, fruits, shoots, stem, leaves, anthers, sepals, petals, pollen, seeds, etc.

The term “tissue” with respect to a plant (or “plant tissue”) means arrangement of multiple plant cells including differentiated and undifferentiated tissues of plants. Plant tissues may constitute part of a plant organ (e.g., the epidermis of a plant leaf) but may also constitute tumor tissues (e.g., callus tissue) and various types of cells in culture (e.g., single cells, protoplasts, embryos, calli, protocorm-like bodies, etc.). Plant tissue may be in planta, in organ culture, tissue culture, or cell culture.

The term “plant” as used herein refers to a plurality of plant cells which are largely differentiated into a structure that is present at any stage of a plant's development. Such structures include one or more plant organs including, but are not limited to, fruit, shoot, stem, leaf, flower petal, etc.

The term “chromosomal DNA” or “chromosomal DNA-sequence” is to be understood as the genomic DNA of the cellular nucleus independent from the cell cycle status. Chromosomal DNA might therefore be organized in chromosomes or chromatids, they might be condensed or uncoiled. An insertion into the chromosomal DNA can be demonstrated and analyzed by various methods known in the art like e.g., PCR analysis, Southern blot analysis, fluorescence in situ hybridization (FISH), and in situ PCR.

The term “structural gene” as used herein is intended to mean a DNA sequence that is transcribed into mRNA which is then translated into a sequence of amino acids characteristic of a specific polypeptide.

The term “expression” refers to the biosynthesis of a gene product. For example, in the case of a structural gene, expression involves transcription of the structural gene into mRNA and—optionally—the subsequent translation of mRNA into one or more polypeptides.

The term “expression cassette” or “expression construct” as used herein is intended to mean the combination of any nucleic acid sequence to be expressed in operable linkage with a promoter sequence and—optionally—additional elements (like e.g., terminator and/or polyadenylation sequences) which facilitate expression of said nucleic acid sequence.

“Promoter”, “promoter element,” or “promoter sequence” as used herein, refers to the nucleotide sequences at the 5′ end of a nucleotide sequence which direct the initiation of transcription (i.e., is capable of controlling the transcription of the nucleotide sequence into mRNA). A promoter is typically, though not necessarily, located 5′ (i.e., upstream) of a nucleotide sequence of interest (e.g., proximal to the transcriptional start site of a structural gene) whose transcription into mRNA it controls, and provides a site for specific binding by RNA polymerase and other transcription factors for initiation of transcription. Promoter sequences are necessary, but not always sufficient, to drive the expression of a downstream gene. In general, eukaryotic promoters include a characteristic DNA sequence homologous to the consensus 5′-TATAAT-3′ (TATA) box about 10-30 bp 5′ to the transcription start (cap) site, which, by convention, is numbered +1. Bases 3′ to the cap site are given positive numbers, whereas bases 5′ to the cap site receive negative numbers, reflecting their distance from the cap site. Another promoter component, the CAAT box, is often found about 30 to 70 bp 5′ to the TATA box and has homology to the canonical form 5′-CCMT-3′ (Breathnach 1981). In plants the CAAT box is sometimes replaced by a sequence known as the AGGA box, a region having adenine residues symmetrically flanking the triplet G(or T)NG (Messing 1983). Other sequences conferring regulatory influences on transcription can be found within the promoter region and extending as far as 1000 bp or more 5′ from the cap site. The term “constitutive” when made in reference to a promoter means that the promoter is capable of directing transcription of an operably linked nucleic acid sequence in the absence of a stimulus (e.g., heat shock, chemicals, light, etc.). Typically, constitutive promoters are capable of directing expression of a transgene in substantially any cell and any tissue.

Regulatory Control refers to the modulation of gene expression induced by DNA sequence elements located primarily, but not exclusively, upstream of (5′ to) the transcription start site. Regulation may result in an all-or-nothing response to environmental stimuli, or it may result in variations in the level of gene expression. In this invention, the heat shock regulatory elements function to enhance transiently the level of downstream gene expression in response to sudden temperature elevation.

Polyadenylation signal refers to any nucleic acid sequence capable of effecting mRNA processing, usually characterized by the addition of polyadenyli6 acid tracts to the 3′-ends of the mRNA precursors. The polyadenylation signal DNA segment may itself be a composite of segments derived from several sources, naturally occurring or synthetic, and may be from a genomic DNA or an RNAderived cDNA. Polyadenylation signals are commonly recognized by the presence of homology to the canonical form 5′-AATAA-3′, although variation of distance, partial “readthrough”, and multiple tandem canonical sequences are not uncommon (Messing 1983). It should be recognized that a canonical “polyadenylation signal” may in fact cause transcriptional termination and not polyadenylation per se (Montell 1983).

Heat shock elements refer to DNA sequences that regulate gene expression in response to the stress of sudden temperature elevations. The response is seen as an immediate albeit transitory enhancement in level of expression of a downstream gene. The original work on heat shock genes was done with Drosophila but many other species including plants (Bameft 1980) exhibited analogous responses to stress. The essential primary component of the heat shock element was described in Drosophila to have the consensus sequence 5′-CTGGAATNTTCTAGA-3′ (where N=A, T, C, or G) and to be located in the region between residues −66 through −47 bp upstream to the transcriptional start site (Pelham 1982). A chemically synthesized oligonucleotide copy of this consensus sequence can replace the natural sequence in conferring heat shock inducibility.

Leader sequence refers to a DNA sequence comprising about 100 nucleotides located between the transcription start site and the translation start site. Embodied within the leader sequence is a region that specifies the ribosome binding site.

Introns or intervening sequences refer in this work to those regions of DNA sequence that are transcribed along with the coding sequences (exons) but are then removed in the formation of the mature mRNA. Introns may occur anywhere within a transcribed sequence—between coding sequences of the same or different genes, within the coding sequence of a gene, interrupting and splitting its amino acid sequences, and within the promoter region (5′ to the translation start site). Introns in the primary transcript are excised and the coding sequences are simultaneously and precisely ligated to form the mature mRNA. The junctions of introns and exons form the splice sites. The base sequence of an intron begins with GU and ends with AG. The same splicing signal is found in many higher eukaryotes.

The term “operable linkage” or “operably linked” is to be understood as meaning, for example, the sequential arrangement of a regulatory element (e.g. a promoter) with a nucleic acid sequence to be expressed and, if appropriate, further regulatory elements (such as e.g., a terminator) in such a way that each of the regulatory elements can fulfill its intended function to allow, modify, facilitate or otherwise influence expression of said nucleic acid sequence. The expression may result depending on the arrangement of the nucleic acid sequences in relation to sense or antisense RNA. To this end, direct linkage in the chemical sense is not necessarily required. Genetic control sequences such as, for example, enhancer sequences, can also exert their function on the target sequence from positions which are further away, or indeed from other DNA molecules. Preferred arrangements are those in which the nucleic acid sequence to be expressed recombinantly is positioned behind the sequence acting as promoter, so that the two sequences are linked covalently to each other. The distance between the promoter sequence and the nucleic acid sequence to be expressed recombinantly is preferably less than 200 base pairs, especially preferably less than 100 base pairs, very especially preferably less than 50 base pairs. Operable linkage, and an expression cassette, can be generated by means of customary recombination and cloning techniques as described (e.g., in Maniatis 1989; Silhavy 1984; Ausubel 1987; Gelvin 1990). However, further sequences which, for example, act as a linker with specific cleavage sites for restriction enzymes, or as a signal peptide, may also be positioned between the two sequences. The insertion of sequences may also lead to the expression of fusion proteins. Preferably, the expression cassette, consisting of a linkage of promoter and nucleic acid sequence to be expressed, can exist in a vectorintegrated form and be inserted into a plant genome, for example by transformation.

The term “transformation” as used herein refers to the introduction of genetic material (e.g., a transgene) into a cell. Transformation of a cell may be stable or transient. The term “transient transformation” or “transiently transformed” refers to the introduction of one or more transgenes into a cell in the absence of integration of the transgene into the host cell's genome. Transient transformation may be detected by, for example, enzymelinked immunosorbent assay (ELISA) which detects the presence of a polypeptide encoded by one or more of the transgenes. Alternatively, transient transformation may be detected by detecting the activity of the protein (e.g., β-glucuronidase) encoded by the transgene (e.g., the uid A gene) as demonstrated herein [e.g., histochemical assay of GUS enzyme activity by staining with X-gluc which gives a blue precipitate in the presence of the GUS enzyme; and a chemiluminescent assay of GUS enzyme activity using the GUSLight kit (Tropix)]. The term “transient transformant” refers to a cell which has transiently incorporated one or more transgenes. In contrast, the term “stable transformation” or “stably transformed” refers to the introduction and integration of one or more transgenes into the genome of a cell, preferably resulting in chromosomal integration and stable heritability through meiosis. Stable transformation of a cell may be detected by Southern blot hybridization of genomic DNA of the cell with nucleic acid sequences which are capable of binding to one or more of the transgenes. Alternatively, stable transformation of a cell may also be detected by the polymerase chain reaction of genomic DNA of the cell to amplify transgene sequences. The term “stable transformant” refers to a cell which has stably integrated one or more transgenes into the genomic DNA (including the DNA of the plastids and the nucleus), preferably integration into the chromosomal DNA of the nucleus. Thus, a stable transformant is distinguished from a transient transformant in that, whereas genomic DNA from the stable transformant contains one or more transgenes, genomic DNA from the transient transformant does not contain a transgene. Transformation also includes introduction of genetic material into plant cells in the form of plant viral vectors involving epichromosomal replication and gene expression which may exhibit variable properties with respect to meiotic stability. Transformation also includes introduction of genetic material into plant cells in the form of plant viral vectors involving epichromosomal replication and gene expression which may exhibit variable properties with respect to meiotic stability. Preferably, the term “transformation” includes introduction of genetic material into plant cells resulting in chromosomal integration and stable heritability through meiosis.

The terms “infecting” and “infection” with a bacterium refer to coincubation of a target biological sample, (e.g., cell, tissue, etc.) with the bacterium under conditions such that nucleic acid sequences contained within the bacterium are introduced into one or more cells of the target biological sample.

The term “Agrobacterium” refers to a soil-borne, Gram-negative, rod-shaped phytopathogenic bacterium which causes crown gall. The term “Agrobacterium” includes, but is not limited to, the strains Agrobacterium tumefaciens, (which typically causes crown gall in infected plants), and Agrobacterium rhizogenes (which causes hairy root disease in infected host plants). Infection of a plant cell with Agrobacterium generally results in the production of opines (e.g., nopaline, agropine, octopine etc.) by the infected cell. Thus, Agrobacterium strains which cause production of nopaline (e.g., strain LBA4301, C58, A208) are referred to as “nopaline-type” Agrobacteria, Agrobacterium strains which cause production of octopine (e.g., strain LBA4404, Ach5, B6) are referred to as “octopine-type” Agrobacteria; and Agrobacterium strains which cause production of agropine (e.g., strain EHA105, EHA101, A281) are referred to as “agropine-type” Agrobacteria.

The terms “bombarding, “bombardment,” and “biolistic bombardment” refer to the process of accelerating particles towards a target biological sample (e.g., cell, tissue, etc.) to effect wounding of the cell membrane of a cell in the target biological sample and/or entry of the particles into the target biological sample. Methods for biolistic bombardment are known in the art (e.g., U.S. Pat. No. 5,584,807, the contents of which are herein incorporated by reference), and are commercially available (e.g., the helium gas-driven microprojectile accelerator (PDS-1000/He) (BioRad)

The term “hybridization” as used herein includes “any process by which a strand of nucleic acid joins with a complementary strand through base pairing.” (Coombs 1994). Hybridization and the strength of hybridization (i.e., the strength of the association between the nucleic acids) is impacted by such factors as the degree of complementarity between the nucleic acids, stringency of the conditions involved, the Tm of the formed hybrid, and the G:C ratio within the nucleic acids.

As used herein, the term “Tm” is used in reference to the “melting temperature.” The melting temperature is the temperature at which a population of double-stranded nucleic acid molecules becomes half dissociated into single strands. The equation for calculating the Tm of nucleic acids is well known in the art. As indicated by standard references, a simple estimate of the Tm value may be calculated by the equation: Tm=81.5+0.41(% G+C), when a nucleic acid is in aqueous solution at 1 M NaCl [see e.g., Anderson and Young, Quantitative Filter Hybridization, in Nucleic Acid Hybridization (1985)]. Other references include more sophisticated computations which take structural as well as sequence characteristics into account for the calculation of Tm.

Low stringency conditions when used in reference to nucleic acid hybridization unless defined elsewhise comprise conditions equivalent to binding or hybridization at 68° C. in a solution consisting of 5×SSPE (43.8 g/L NaCl, 6.9 g/L NaH₂PO₄.H₂O and 1.85 g/L EDTA, pH adjusted to 7.4 with NaOH), 1% SDS, 5× Denhardt's reagent [50×Denhardt's contains the following per 500 mL: 5 g Ficoll (Type 400, Pharmacia), 5 g BSA (Fraction V; Sigma)] and 100 μg/mL denatured salmon sperm DNA followed by washing in a solution comprising 0.2×SSPE, and 0.1% SDS at room temperature when a DNA probe of about 100 to about 1000 nucleotides in length is employed.

High stringency conditions when used in reference to nucleic acid hybridization comprise unless defined elsewhise conditions equivalent to binding or hybridization at 68° C. in a solution consisting of 5×SSPE, 1% SDS, 5×Denhardt's reagent and 100 μg/mL denatured salmon sperm DNA followed by washing in a solution comprising 0.1×SSPE, and 0.1% SDS at 68° C. when a probe of about 100 to about 1000 nucleotides in length is employed.

The term “equivalent” when made in reference to a hybridization condition as it relates to a hybridization condition of interest means that the hybridization condition and the hybridization condition of interest result in hybridization of nucleic acid sequences which have the same range of percent (%) homology. For example, if a hybridization condition of interest results in hybridization of a first nucleic acid sequence with other nucleic acid sequences that have from 80% to 90% homology to the first nucleic acid sequence, then another hybridization condition is said to be equivalent to the hybridization condition of interest if this other hybridization condition also results in hybridization of the first nucleic acid sequence with the other nucleic acid sequences that have from 80% to 90% homology to the first nucleic acid sequence.

When used in reference to nucleic acid hybridization the art knows well that numerous equivalent conditions may be employed to comprise either low or high stringency conditions; factors such as the length and nature (DNA, RNA, base composition) of the probe and nature of the target (DNA, RNA, base composition, present in solution or immobilized, etc.) and the concentration of the salts and other components (e.g., the presence or absence of formamide, dextran sulfate, polyethylene glycol) are considered and the hybridization solution may be varied to generate conditions of either low or high stringency hybridization different from, but equivalent to, the above-listed conditions. Those skilled in the art know that whereas higher stringencies may be preferred to reduce or eliminate non-specific binding, lower stringencies may be preferred to detect a larger number of nucleic acid sequences having different homologies.

### DETAILED DESCRIPTION OF THE INVENTION

The present invention may be understood more readily by reference to the following detailed description of the preferred embodiments of the invention and the Examples included therein.

Before the present compounds, compositions, and methods are disclosed and described, it is to be understood that this invention is not limited to specific nucleic acids, specific polypeptides, specific cell types, specific host cells, specific conditions, or specific methods, etc., as such may, of course, vary, and the numerous modifications and variations therein will be apparent to those skilled in the art. It is also to be understood that the terminology used herein is for the purpose of describing particular embodiments only and is not intended to be limiting. As used in the specification and in the claims, “a” or “an” can mean one or more, depending upon the context in which it is used. Thus, for example, reference to “a cell” can mean that at least one cell can be utilized.

The present invention is based, in part, on the isolation and characterization of nucleic acid molecules encoding KCS-like LMPs from plants including canola (Brassica napus), soybean (Glycine max and linseed (Linum usitatissimum) and other related crop species like rice, wheat, maize, barley, linseed, sugar beat or sunflower.

In accordance with the purpose(s) of this invention, as embodied and broadly described herein, this invention, in one aspect, provides an isolated nucleic acid from a plant (Brassica napus, Glycine max or Linum usitatissimum) encoding a Lipid Metabolism Protein (LMP), or a portion thereof.

One aspect of the invention pertains to isolated nucleic acid molecules that encode LMP polypeptides or biologically active portions thereof, as well as nucleic acid fragments sufficient for use as hybridization probes or primers for the identification or amplification of an LMP-encoding nucleic acid (e.g., LMP DNA). As used herein, the term “nucleic acid molecule” is intended to include DNA molecules (e.g., cDNA or genomic DNA) and RNA molecules (e.g., mRNA) and analogs of the DNA or RNA generated using nucleotide analogs. This term also encompasses untranslated sequence located at both the 3′ and 5′ ends of the coding region of a gene: at least about 1000 nucleotides of sequence upstream from the 5′ end of the coding region and at least about 200 nucleotides of sequence downstream from the 3′ end of the coding region of the gene. The nucleic acid molecule can be single-stranded or double-stranded, but preferably is double-stranded DNA. An “isolated” nucleic acid molecule is one, which is substantially separated from other nucleic acid molecules, which are present in the natural source of the nucleic acid. Preferably, an “isolated” nucleic acid is substantially free of sequences that naturally flank the nucleic acid (i.e., sequences located at the 5′ and 3′ ends of the nucleic acid) in the genomic DNA of the organism, from which the nucleic acid is derived. For example, in various embodiments, the isolated LMP nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived (e.g., a Brassica napus, Glycine max or Linum usitatissimum cell). Moreover, an “isolated” nucleic acid molecule, such as a cDNA molecule, can be substantially free of other cellular material, or culture medium when produced by recombinant techniques, or chemical precursors or other chemicals when chemically synthesized.

A nucleic acid molecule of the present invention, e.g., a nucleic acid molecule having a nucleotide sequence of Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23, or a portion thereof, can be isolated using standard molecular biology techniques and the sequence information provided herein. For example, an Brassica napeus, Glycine max or Linum usitatissimum LMP cDNA can be isolated from an Brassica napus, Glycine max or Linum usitatissimum library using all or portion of one of the sequences of Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23, as a hybridization probe and standard hybridization techniques (e.g., as described in Sambrook et al. 1989, Molecular Cloning A Laboratory Manual 2nd, ed., Cold Spring Harbor Laboratory, Cold Spring Harbor Laboratory Press, Cold Spring Harbor, N.Y.). Moreover, a nucleic acid molecule encompassing all or a portion of one of the sequences of Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23, can be isolated by the polymerase chain reaction using oligonucleotide primers designed based upon this sequence (e.g., a nucleic acid molecule encompassing all or a portion of one of the sequences of Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23, can be isolated by the polymerase chain reaction using oligonucleotide primers designed based upon this same sequence of Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23,). For example, mRNA can be isolated from plant cells (e.g., by the guanidinium-thiocyanate extraction procedure of Chirgwin et al. 1979, Biochemistry 18:5294-5299) and cDNA can be prepared using reverse transcriptase (e.g., Moloney MLV reverse transcriptase, available from Gibco/BRL, Bethesda, Md.; or AMV reverse transcriptase, available from Seikagaku America, Inc., St. Petersburg, Fla.). Synthetic oligonucleotide primers for polymerase chain reaction amplification can be designed based upon one of the nucleotide sequences shown in Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23. A nucleic acid of the invention can be amplified using cDNA or, alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to a LMP nucleotide sequence can be prepared by standard synthetic techniques, e.g., using an automated DNA synthesizer.

In a preferred embodiment, an isolated nucleic acid of the invention comprises one of the nucleotide sequences shown in Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23. The sequences of Appendix A, in a preferred embodiment as depicted in SEQ ID NO: 1, SEQ ID NO: 5, SEQ ID NO: 9, SEQ ID NO: 13, SEQ ID NO: 17 or SEQ ID NO: 21, correspond to the Brassica napus, Glycine max or Linum usitatissimum LMP cDNAs of the invention. These cDNAs comprise sequences encoding LMPs (i.e., the “coding region”, indicated in Appendix A), as well as 5′ untranslated sequences and 3′ untranslated sequences. Alternatively, the nucleic acid molecules can comprise only the coding region of any of the sequences in Appendix A, in a preferred embodiment as depicted in SEQ ID NO: 3, SEQ ID NO: 7, SEQ ID NO: 11, SEQ ID NO: 15, SEQ ID NO: 19, or SEQ ID NO: 23, or can contain whole genomic fragments isolated from genomic DNA.

For the purposes of this application, it will be understood that each of the sequences set forth in Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23, has an identifying entry number (e.g., Bn 46783084). Each of these sequences may generally comprise three parts: a 5′ upstream region, a coding region, and a downstream region. A coding region of these sequences is indicated as “ORF positon” (Table 3).

In another preferred embodiment, an isolated nucleic acid molecule of the invention comprises a nucleic acid molecule, which is a complement of one of the nucleotide sequences shown in Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23, or a portion thereof. A nucleic acid molecule which is complementary to one of the nucleotide sequences shown in Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23, is one which is sufficiently complementary to one of the nucleotide sequences shown in Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23, such that it can hybridize to one of the nucleotide sequences shown in Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23, thereby forming a stable duplex.

In still another preferred embodiment, an isolated nucleic acid molecule of the invention comprises a nucleotide sequence which is at least about 50-60%, preferably at least about 60-70%, more preferably at least about 70-80%, 80-90%, or 90-95%, that means preferably 70%, 72.5%, 75%, 77.5%, 80%, 82.5%, 85%, 87.5%, 90% or 92.5% or more and even more preferably at least about 95%, 96%, 97%, 98%, 99% or more homologous to a nucleotide sequence shown in Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23, or a portion thereof. In an additional preferred embodiment, an isolated nucleic acid molecule of the invention comprises a nucleotide sequence which hybridizes, e.g., hybridizes under stringent conditions, to one of the nucleotide sequences shown in Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23, or a portion thereof. These hybridization conditions include washing with a solution having a salt concentration of about 0.02 molar at pH 7 at about 60° C.

Moreover, the nucleic acid molecule of the invention can comprise only a portion of the coding region of one of the sequences in Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23, for example a fragment, which can be used as a probe or primer or a fragment encoding a biologically active portion of a LMP. The nucleotide sequences determined from the cloning of the LMP genes from Brassica napus, Glycine max or Linum usitatissimum allows for the generation of probes and primers designed for use in identifying and/or cloning LMP homologues in other cell types and organisms, as well as LMP homologues from other plants or related species. Therefore this invention also provides compounds comprising the nucleic acids disclosed herein, or fragments thereof. These compounds include the nucleic acids attached to a moiety. These moieties include, but are not limited to, detection moieties, hybridization moieties, purification moieties, delivery moieties, reaction moieties, binding moieties, and the like. The probe/primer typically comprises substantially purified oligonucleotide. The oligonucleotide typically comprises a region of nucleotide sequence that hybridizes under stringent conditions to at least about 12, preferably about 25, more preferably about 40, 50 or 75 consecutive nucleotides of a sense strand of one of the sequences set forth in Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23, an anti-sense sequence of one of the sequences set forth in Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23, or naturally occurring mutants thereof. Primers based on a nucleotide sequence of Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23, can be used in PCR reactions to clone LMP homologues. Probes based on the LMP nucleotide sequences can be used to detect transcripts or genomic sequences encoding the same or homologous proteins. In preferred embodiments, the probe further comprises a label group attached thereto, e.g. the label group can be a radioisotope, a fluorescent compound, an enzyme, or an enzyme co-factor. Such probes can be used as a part of a genomic marker test kit for identifying cells which express a LMP, such as by measuring a level of a LMP-encoding nucleic acid in a sample of cells, e.g., detecting LMP mRNA levels or determining whether a genomic LMP gene has been mutated or deleted.

In one embodiment, the nucleic acid molecule of the invention encodes a protein or portion thereof which includes an amino acid sequence which is sufficiently homologous to an amino acid encoded by a sequence of Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23, such that the protein or portion thereof maintains the same or a similar function as the wild-type protein. As used herein, the language “sufficiently homologous” refers to proteins or portions thereof which have amino acid sequences which include a minimum number of identical or equivalent (e.g., an amino acid residue, which has a similar side chain as an amino acid residue in one of the ORFs of a sequence of Appendix A) amino acid residues to an amino acid sequence such that the protein or portion thereof is able to participate in the metabolism of compounds necessary for the production of seed storage compounds in plants, construction of cellular membranes in microorganisms or plants, or in the transport of molecules across these membranes. Examples of LMP-encoding nucleic acid sequences are set forth in Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23.

As altered or increased sugar and/or fatty acid production is a general trait wished to be inherited into a wide variety of plants like maize, wheat, rye, oat, triticale, rice, barley, soybean, peanut, cotton, canola, manihot, pepper, sunflower, sugar beet and tagetes, solanaceous plants like potato, tobacco, eggplant, and tomato, Vicia species, pea, alfalfa, bushy plants (coffee, cacao, tea), Salix species, trees (oil palm, coconut) and perennial grasses and forage crops, these crop plants are also preferred target plants for genetic engineering as one further embodiment of the present invention.

Portions of proteins encoded by the LMP nucleic acid molecules of the invention are preferably biologically active portions of one of the LMPs. As used herein, the term “biologically active portion of a LMP” is intended to include a portion, e.g., a domain/motif, of a LMP that participates in the metabolism of compounds necessary for the biosynthesis of seed storage lipids, or the construction of cellular membranes in microorganisms or plants, or in the transport of molecules across these membranes, or has an activity as set forth in Table 3. To determine whether a LMP or a biologically active portion thereof can participate in the metabolism of compounds necessary for the production of seed storage compounds and cellular membranes, an assay of enzymatic activity may be performed. Such assay methods are well known to those skilled in the art, and as described in Example 14 of the Exemplification.

Biologically active portions of a LMP include peptides comprising amino acid sequences derived from the amino acid sequence of a LMP (e.g., an amino acid sequence encoded by a nucleic acid of Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23, or the amino acid sequence of a protein homologous to a LMP, which include fewer amino acids than a full length LMP or the full length protein which is homologous to a LMP) and exhibit at least one activity of a LMP. Typically, biologically active portions (peptides, e.g., peptides which are, for example, 5, 10, 15, 20, 30, 35, 36, 37, 38, 39, 40, 50, 100 or more amino acids in length) comprise a domain or motif with at least one activity of a LMP. Moreover, other biologically active portions, in which other regions of the protein are deleted, can be prepared by recombinant techniques and evaluated for one or more of the activities described herein. Preferably, the biologically active portions of a LMP include one or more selected domains/motifs or portions thereof having biological activity.

Additional nucleic acid fragments encoding biologically active portions of a LMP can be prepared by isolating a portion of one of the sequences, expressing the encoded portion of the LMP or peptide (e.g., by recombinant expression in vitro) and assessing the activity of the encoded portion of the LMP or peptide.

The invention further encompasses nucleic acid molecules that differ from one of the nucleotide sequences shown in Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23, (and portions thereof due to degeneracy of the genetic code and thus encode the same LMP as that encoded by the nucleotide sequences shown in Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23. In a further embodiment, the nucleic acid molecule of the invention encodes a full length protein which is substantially homologous to an amino acid sequence of a polypeptide encoded by an open reading frame shown in Appendix A. In one embodiment, the full-length nucleic acid or protein or fragment of the nucleic acid or protein is from Brassica napus, Glycine max or Linum usitatissimum.

In addition to the Brassica napus, Glycine max or Linum usitatissimum LMP nucleotide sequences shown in Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23, it will be appreciated by those skilled in the art that DNA sequence polymorphisms that lead to changes in the amino acid sequences of LMPs may exist within a population (e.g., the Brassica napus, Glycine max or Linum usitatissimum population). Such genetic polymorphism in the LMP gene may exist among individuals within a population due to natural variation. As used herein, the terms “gene” and “recombinant gene” refer to nucleic acid molecules comprising an open reading frame encoding a LMP, preferably a Brassica napus, Glycine max or Linum usitatissimum LMP. Such natural variations can typically result in 140% variance in the nucleotide sequence of the LMP gene. Any and all such nucleotide variations and resulting amino acid polymorphisms in LMP that are the result of natural variation and that do not alter the functional activity of LMPs are intended to be within the scope of the invention.

Nucleic acid molecules corresponding to natural variants and non-Brassica napus, Glycine max or Linum usitatissimum orthologs of the Brassica napus, Glycine max or Linum usitatissimum LMP cDNA of the invention can be isolated based on their homology to Brassica napus, Glycine max or Linum usitatissimum LMP nucleic acid disclosed herein using the Brassica napus, Glycine max or Linum usitatissimum cDNA, or a portion thereof, as a hybridization probe according to standard hybridization techniques under stringent hybridization conditions. As used herein, the term “orthologs” refers to two nucleic acids from different species, but that have evolved from a common ancestral gene by speciation. Normally, orthologs encode proteins having the same or similar functions. Accordingly, in another embodiment, an isolated nucleic acid molecule of the invention is at least 15 nucleotides in length and hybridizes under stringent conditions to the nucleic acid molecule comprising a nucleotide sequence of Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23. In other embodiments, the nucleic acid is at least 30, 50, 100, 250 or more nucleotides in length. As used herein, the term “hybridizes under stringent conditions” is intended to describe conditions for hybridization and washing under which nucleotide sequences at least 60% homologous to each other typically remain hybridized to each other. Preferably, the conditions are such that sequences at least about 65%, more preferably at least about 70%, and even more preferably at least about 75% or more homologous to each other typically remain hybridized to each other. Such stringent conditions are known to those skilled in the art and can be found in Current Protocols in Molecular Biology, John Wiley & Sons, N.Y., 1989: 6.3.1-6.3.6. A preferred, non-limiting example of stringent hybridization conditions are hybridization in 6× sodium chloride/sodium citrate (SSC) at about 45° C., followed by one or more washes in 0.2×SSC, 0.1% SDS at 50-65° C. Preferably, an isolated nucleic acid molecule of the invention that hybridizes under stringent conditions to a sequence of Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23, corresponds to a naturally occurring nucleic acid molecule. As used herein, a “naturallyoccurring” nucleic acid molecule refers to an RNA or DNA molecule having a nucleotide sequence that occurs in nature (e.g., encodes a natural protein). In one embodiment, the nucleic acid encodes a natural Brassica napus, Glycine max or Linum usitatissimum LMP.

In addition to naturally-occurring variants of the LMP sequence that may exist in the population, the skilled artisan will further appreciate that changes can be introduced by mutation into a nucleotide sequence of Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23, thereby leading to changes in the amino acid sequence of the encoded LMP, without altering the functional ability of the LMP. For example, nucleotide substitutions leading to amino acid substitutions at “non-essential” amino acid residues can be made in a sequence of Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23. A “non-essental” amino acid residue is a residue that can be altered from the wild-type sequence of one of the LMPs (Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23) without altering the activity of said LMP, whereas an “essential” amino acid residue is required for LMP activity. Other amino acid residues, however, (e.g., those that are not conserved or only semi-conserved in the domain having LMP activity) may not be essential for activity and thus are likely to be amenable to alteration without altering LMP activity.

Accordingly, another aspect of the invention pertains to nucleic acid molecules encoding LMPs that contain changes in amino acid residues that are not essential for LMP activity. Such LMPs differ in amino acid sequence from a sequence yet retain at least one of the LMP activities described herein. In one embodiment, the isolated nucleic acid molecule comprises a nucleotide sequence encoding a protein, wherein the protein comprises an amino acid sequence at least about 50% homologous to an amino acid sequence encoded by a nucleic acid of Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23, and is capable of participation in the metabolism of compounds necessary for the production of seed storage compounds in Brassica napus, Glycine max or Linum usitatissimum, or cellular membranes, or has one or more activities set forth in Table 3. Preferably, the protein encoded by the nucleic acid molecule is at least about 50-60% homologous to one of the sequences encoded by a nucleic acid of Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23, more preferably at least about 60-70% homologous to one of the sequences encoded by a nucleic acid of Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23, even more preferably at least about 70-80%, 80-90%, 90-95%, that means preferably 70%, 72.5%, 75%, 77.5%, 80%, 82.5%, 85%, 87.5%, 90% or 92.5% or more and in an even more preferred embodiment 95%, 96%, 97%, 98% or 99% or more homologous to one of the sequences encoded by a nucleic acid of Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23, and most preferably at least about 96%, 97%, 98%, or 99% homologous to one of the sequences encoded by a nucleic acid of Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23.

To determine the percent homology of two amino acid sequences (e.g., one of the sequences encoded by a nucleic acid of Appendix A and a mutant form thereof) or of two nucleic acids, the sequences are aligned for optimal comparison purposes (e.g., gaps can be introduced in the sequence of one protein or nucleic acid for optimal alignment with the other protein or nucleic acid). The amino acid residues or nucleotides at corresponding amino acid positions or nucleotide positions are then compared. When a position in one sequence (e.g., one of the sequences encoded by a nucleic acid of Appendix A) is occupied by the same amino acid residue or nucleotide as the corresponding position in the other sequence (e.g., a mutant form of the sequence selected from the polypeptide encoded by a nucleic acid of Appendix A), then the molecules are homologous at that position (i.e., as used herein amino acid or nucleic acid “homology” is equivalent to amino acid or nucleic acid “identity”). The percent homology between the two sequences is a function of the number of identical positions shared by the sequences (i.e., % homology=numbers of identical positions/total numbers of positions×100).

An isolated nucleic acid molecule encoding a LMP homologous to a protein sequence encoded by a nucleic acid of Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23, can be created by introducing one or more nucleotide substitutions, additions or deletions into a nucleotide sequence of Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23, such that one or more amino acid substitutions, additions or deletions are introduced into the encoded protein. Mutations can be introduced into one of the sequences of Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23, by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in a LMP is preferably replaced with another amino acid residue from the same side chain family. Alternatively, in another embodiment, mutations can be introduced randomly along all or part of a LMP coding sequence, such as by saturation mutagenesis, and the resultant mutants can be screened for a LMP activity described herein to identify mutants that retain LMP activity. Following mutagenesis of one of the sequences of Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23, the encoded protein can be expressed recombinantly and the activity of the protein can be determined using, for example, assays described herein (see Examples 11-13 of the Exemplificabon).

LMPs are preferably produced by recombinant DNA techniques. For example, a nucleic acid molecule encoding the protein is cloned into an expression vector (as described above), the expression vector is introduced into a host cell (as described herein) and the LMP is expressed in the host cell. The LMP can then be isolated from the cells by an appropriate purification scheme using standard protein purification techniques. Alternative to recombinant expression, a LMP or peptide thereof can be synthesized chemically using standard peptide synthesis techniques. Moreover, native LMP can be isolated from cells, for example using an antiLMP antibody, which can be produced by standard techniques utilizing a LMP or fragment thereof of this invention.

The invention also provides LMP chimeric or fusion proteins. As used herein, a LMP “chimeric protein” or “fusion protein” comprises a LMP polypeptide operatively linked to a non-LMP polypeptide. An “LMP polypeptide” refers to a polypeptide having an amino acid sequence corresponding to a LMP, whereas a “non-LMP polypeptide” refers to a polypeptide having an amino acid sequence corresponding to a protein which is not substantially homologous to the LMP, e.g., a protein which is different from the LMP and which is derived from the same or a different organism. Within the fusion protein, the term “operatively linked” is intended to indicate that the LMP polypeptide and the non-LMP polypeptide are fused to each other so that both sequences fulfill the proposed function attributed to the sequence used. The non-LMP polypeptide can be fused to the N-terminus or C-terminus of the LMP polypeptide. For example, in one embodiment, the fusion protein is a GST-LMP (glutathione S-transferase) fusion protein in which the LMP sequences are fused to the C-terminus of the GST sequences. Such fusion proteins can facilitate the purification of recombinant LMPs. In another embodiment, the fusion protein is a LMP containing a heterologous signal sequence at its N-terminus. In certain host cells (e.g., mammalian host cells), expression and/or secretion of a LMP can be increased through use of a heterologous signal sequence.

Preferably, a LMP chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments, which can subsequently be annealed and reamplified to generate a chimeric gene sequence (see, for example, Current Protocols in Molecular Biology, eds. Ausubel et al., John Wiley & Sons: 1992). Moreover, many expression vectors are commercially available that already encode a fusion moiety (e.g., a GST polypeptide). An LMP-encoding nucleic acid can be cloned into such an expression vector such that the fusion moiety is linked in-frame to the LMP.

In addition to the nucleic acid molecules encoding LMPs described above, another aspect of the invention pertains to isolated nucleic acid molecules that are antisense thereto. An “antisense” nucleic acid comprises a nucleotide sequence that is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire LMP coding strand, or to only a portion thereof. In one embodiment, an antisense nucleic acid molecule is antisense to a “coding region” of the coding strand of a nucleotide sequence encoding a LMP. The term “coding region” refers to the region of the nucleotide sequence comprising codons that are translated into amino acid residues (e.g., the entire coding region of Bn46783084 comprises nucleotides 1 to 1491). In another embodiment, the antisense nucleic acid molecule is antisense to a “noncoding region” of the coding strand of a nucleotide sequence encoding LMP. The term “noncoding region” refers to 5′ and 3′ sequences that flank the coding region that are not translated into amino acids (i.e., also referred to as 5′ and 3′ untranslated regions).

Given the coding strand sequences encoding LMP disclosed herein (e.g., the sequences set forth in Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23), antisense nucleic acids of the invention can be designed according to the rules of Watson and Crick base pairing. The antisense nucleic acid molecule can be complementary to the entire coding region of LMP mRNA, but more preferably is an oligonucleotide that is antisense to only a portion of the coding or noncoding region of LMP mRNA. For example, the antisense oligonucleotide can be complementary to the region surrounding the translation start site of LMP mRNA. An antisense oligonucleotide can be, for example, about 5, 10, 15, 20, 25, 30, 35, 40, 45 or 50 nucleotides in length. An antisense or sense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5(carboxyhydroxylmethyl) uracil, 5-carboxymethylamino-methyl-2-thiouridine, 5-carboxymethylaminomethyluracil, dihydro-uracil, beta-D-galactosylqueosine, inosine, N-6-isopentenyladenine, 1-methyl-guanine, 1-methylinosine, 2,2-dimethylguanine, 2-methyladenine, 2-methylguanine, 3-methylcytosine, 5-methylcytosine, N-6-adenine, 7-methylguanine, 5-methyl-aminomethyluracil, 5-methoxyamino-methyl-2-thiouracil, beta-D-mannosylqueosine, 5′-methoxycarboxymethyl-uracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diamino-purine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

In another variation of the antisense technology, a double-strand interfering RNA construct can be used to cause a down-regulation of the LMP mRNA level and LMP activity in transgenic plants. This requires transforming the plants with a chimeric construct containing a portion of the LMP sequence in the sense orientation fused to the antisense sequence of the same portion of the LMP sequence. A DNA linker region of variable length can be used to separate the sense and antisense fragments of LMP sequences in the construct.

The antisense nucleic acid molecules of the invention are typically administered to a cell or generated in situ such that they hybridize with or bind to cellular mRNA and/or genomic DNA encoding a LMP to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. The antisense molecule can be modified such that it specifically binds to a receptor or an antigen expressed on a selected cell surface, e.g., by linking the antisense nucleic acid molecule to a peptide or an antibody which binds to a cell surface receptor or antigen. The antisense nucleic acid molecule can also be delivered to cells using the vectors described herein. To achieve sufficient intracellular concentrations of the antisense molecules, vector constructs in which the antisense nucleic acid molecule is placed under the control of a strong prokaryotic, viral, or eukaryotic including plant promoters are preferred.

In yet another embodiment, the antisense nucleic acid molecule of the invention is an-anomeric nucleic acid molecule. An anomeric nucleic acid molecule forms specific double-stranded hybrids with complementary RNA in which, contrary to the usual units, the strands run parallel to each other (Gaultier et al. 1987, Nucleic Acids Res. 15:6625-6641). The antisense nucleic acid molecule can also comprise a 2′-o-methyl-ribonucleotide (Inoue et al. 1987, Nucleic Acids Res. 15:6131-6148) or a chimeric RNA-DNA analogue (Inoue et al. 1987, FEBS Lett. 215:327-330).

In still another embodiment, an antisense nucleic acid of the invention is a ribozyme. Ribozymes are catalytic RNA molecules with ribonuclease activity, which are capable of cleaving a single-stranded nucleic acid, such as an mRNA, to which they have a complementary region. Thus, ribozymes (e.g., hammerhead ribozymes (described in Haselhoff & Gerlach 1988, Nature 334:585-591)) can be used to catalytically cleave LMP mRNA transcripts to thereby inhibit translation of LMP mRNA. A ribozyme having specificity for a LMP-encoding nucleic acid can be designed based upon the nucleotide sequence of a LMP cDNA disclosed herein (i.e., Bn01 in Appendix A) or on the basis of a heterologous sequence to be isolated according to methods taught in this invention. For example, a derivative of a Tetrahymena L-19 IVS RNA can be constructed in which the nucleotide sequence of the active site is complementary to the nucleotide sequence to be cleaved in a LMP-encoding mRNA (see, e.g., Cech et al., U.S. Pat. No. 4,987,071 and Cech et al., U.S. Pat. No. 5,116,742). Alternatively, LMP mRNA can be used to select a catalytic RNA having a specific ribonuclease activity from a pool of RNA molecules (see, e.g., Bartel, D. & Szostak J. W. 1993, Science 261:1411-1418).

Alternatively, LMP gene expression can be inhibited by targeting nucleotide sequences complementary to the regulatory region of a LMP nucleotide sequence (e.g., a LMP promoter and/or enhancers) to form triple helical structures that prevent transcription of a LMP gene in target cells (See generally, Helene C. 1991, Anticancer Drug Des. 6:569-84; Helene C. et al. 1992, Ann. N.Y. Acad. Sci. 660:27-36; and Maher, L. J. 1992, Bioassays 14:807-15).

Another aspect of the invention pertains to vectors, preferably expression vectors, containing a nucleic acid encoding a LMP (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adenoassociated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence and both sequences are fused to each other so that each fulfills its proposed function (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990) or see: Gruber and Crosby, in: Methods in Plant Molecular Biology and Biotechnology, CRC Press, Boca Raton, Fla., eds.: Glick & Thompson, Chapter 7, 89-108 including the references therein. Regulatory sequences include those that direct constitutive expression of a nucleotide sequence in many types of host cell and those that direct expression of the nucleotide sequence only in certain host cells or under certain conditions. It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., LMPs, mutant forms of LMPs, fusion proteins, etc.).

The recombinant expression vectors of the invention can be designed for expression of LMPs in prokaryotic or eukaryotic cells. For example, LMP genes can be expressed in bacterial cells, insect cells (using baculovirus expression vectors), yeast and other fungal cells (see Romanos M. A. et al. 1992, Foreign gene expression in yeast: a review, Yeast 8:423-488; van den Hondel, C. A. M. J. J. et al. 1991, Heterologous gene expression in filamentous fungi, in: More Gene Manipulations in Fungi, Bennet & Lasure, eds., p. 396428:Academic Press: an Diego; and van den Hondel & Punt 1991, Gene transfer systems and vector development for filamentous fungi, in: Applied Molecular Genetics of Fungi, Peberdy et al., eds., p. 1-28, Cambridge University Press: Cambridge), algae (Falciatore et al. 1999, Marine Biotechnology 1:239-251), ciliates of the types: Holotrichia, Peritrichia, Spirotrichia, Suctoria, Tetrahymena, Paramecium, Colpidium, Glaucoma, Platyophrya, Potomacus, Pseudocohnilembus, Euplotes, Engelmaniella, and Stylonychia, especially of the genus Stylonychia lemnae with vectors following a transformation method as described in WO 98/01572 and multicellular plant cells (see Schmidt & Willmitzer 1988, High efficiency Agrobacterium tumefaciens mediated transformation of Arabidopsis thaliana leaf and cotyledon plants, Plant Cell Rep.:583-586); Plant Molecular Biology and Biotechnology, C Press, Boca Raton, Fla., chapter 6/7, S.71-119 (1993); White, Jenes et al., Techniques for Gene Transfer, in: Trans-genic Plants, Vol. 1, Engineering and Utilization, eds.: Kung and Wu, Academic Press 1993, 12843; Potrykus 1991, Annu. Rev. Plant Physiol. Plant Mol. Biol. 42:205-225 (and references cited therein) or mammalian cells. Suitable host cells are discussed further in Goeddel, Gene Expression Technology: Methods in Enzymology 185, Academic Press, San Diego, Calif. 1990). Alternatively, the recombinant expression vector can be transcribed and translated in vitro, for example using T7 promoter regulatory sequences and T7 polymerase.

Expression of proteins in prokaryotes is most often carried out with vectors containing constitutive or inducible promoters directing the expression of either fusion or non-fusion proteins. Fusion vectors add a number of amino acids to a protein encoded therein, usually to the amino terminus of the recombinant protein but also to the C-terminus or fused within suitable regions in the proteins. Such fusion vectors typically serve one or more of the following purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety sub-sequent to purification of the fusion protein. Such enzymes, and their cognate recognition sequences, include Factor Xa, thrombin and enterokinase.

Typical fusion expression vectors include pGEX (Pharmacia Biotech Inc; Smith & Johnson 1988, Gene 67:3140), pMAL (New England Biolabs, Beverly, Mass.) and pRIT5 (Pharmacia, Piscataway, N.J.) which fuse glutathione S-transferase (GST), maltose. E binding protein, or protein A, respectively, to the target recombinant protein. In one embodiment, the coding sequence of the LMP is cloned into a pGEX expression vector to create a vector encoding a fusion protein comprising, from the N-terminus to the C-terminus, GST-thrombin cleavage site-X protein. The fusion protein can be purified by affinity chromatography using glutathione-agarose resin. Recombinant LMP unfused to GST can be recovered by cleavage of the fusion protein with thrombin.

Examples of suitable inducible non-fusion E. coli expression vectors include pTrc (Amann et al. 1988, Gene 69:301-315) and pET 11d (Studier et al. 1990, Gene Expression Technology: Methods in Enzymology 185, Academic Press, San Diego, Calif. 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21 (DE3) or HMS174 (DE3) from a resident prophage harboring a T7 gn1 gene under the transcriptional control of the lacUV 5 promoter.

One strategy to maximize recombinant protein expression is to express the protein in a host bacteria with an impaired capacity to proteolytically cleave the recombinant protein (Gottesman S. 1990, Gene Expression Technology: Methods in Enzymology 185:119-128, Academic Press, San Diego, Calif.). Another strategy is to alter the nucleic acid sequence of the nucleic acid to be inserted into an expression vector so that the individual codons for each amino acid are those preferentially utilized in the bacterium chosen for expression (Wada et al. 1992, Nucleic Acids Res. 20:2111-2118). Such alteration of nucleic acid sequences of the invention can be carried out by standard DNA synthesis techniques.

In another embodiment, the LMP expression vector is a yeast expression vector. Examples of vectors for expression in yeast S. cerevisiae include pYepSec1 (Baldari et al. 1987, Embo J. 6:229-234), pMFa (Kurjan & Herskowitz 1982, Cell 30:933-943), pJRY88 (Schultz et al. 1987, Gene 54:113-123), and pYES2 (Invitrogen Corporation, San Diego, Calif.). Vectors and methods for the construction of vectors appropriate for use in other fungi, such as the filamentous fungi, include those detailed in: van den Hondel & Punt 1991, “Gene transfer systems and vector development for filamentous fungi, in: Applied Molecular Genetics of Fungi, Peberdy et al., eds., p. 1-28, Cambridge University Press: Cambridge.

Alternatively, the LMPs of the invention can be expressed in insect cells using baculovirus expression vectors. Baculovirus vectors available for expression of proteins in cultured insect cells (e.g., Sf 9 cells) include the pAc series (Smith et al. 1983, Mol. Cell. Biol. 3:2156-2165) and the pVL series (Lucklow & Summers 1989, Virology 170:31-39).

In yet another embodiment, a nucleic acid of the invention is expressed in mammalian cells using a mammalian expression vector. Examples of mammalian expression vectors include pCDM8 (Seed 1987, Nature 329:840) and pMT2PC (Kaufman et al. 1987, EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, Fritsh and Maniabs, Molecular Cloning: A Laboratory Manual 2nd, ed., Cold Spring Harbor Laboratory, Cold Spring Harbor Laboratory Press, Cold Spring Harbor, NY, 1989.

In another embodiment, the LMPs of the invention may be expressed in uni-cellular plant cells (such as algae, see Falciatore et al. (1999, Marine Biotechnology 1:239-251 and references therein) and plant cells from higher plants (e.g., the spermatophytes, such as crop plants). Examples of plant expression vectors include those detailed in: Becker, Kemper, Schell and Masterson (1992, “New plant binary vectors with selectable markers located, proximal to the left border”, Plant Mol. Biol. 20:1195-1197) and Bevan (1984, “Binary Agrobacterium vectors for plant transformation, Nucleic Acids Res. 12:8711-8721; Vectors for Gene Transfer in Higher Plants; in: Transgenic Plants, Vol. 1, Engineering and Utilization, eds.: Kung und R. Wu, Academic Press, 1993, S. 15-38).

A plant expression cassette preferably contains regulatory sequences capable to drive gene expression in plant cells and which are operably linked so that each sequence can fulfil its function such as termination of transcription, including polyadenylation signals. Preferred polyadenylation signals are those originating from Agrobacterium tumefaciens t-DNA such as the gene 3 known as octopine synthase of the Ti-plasmid pTiACH5 (Gielen et al. 1984, EMBO J. 3:835) or functional equivalents thereof but also all other terminators functionally active in plants are suitable.

As plant gene expression is very often not limited on transcriptional levels a plant expression cassette preferably contains other operably linked sequences like translational enhancers such as the overdrive-sequence containing the 5′-untranslated leader sequence from tobacco mosaic virus enhancing the protein per RNA ratio (Gallie et al. 1987, Nucleic Acids Res. 15:8693-8711).

Plant gene expression has to be operably linked to an appropriate promoter conferring gene expression in a timely, cell or tissue specific manner. Preferred are promoters driving consecutive expression (Benfey et al. 1989, EMBO J. 8:2195-2202) like those derived from plant viruses like the 35S CAMV (Franck et al. 1980, Cell 21:285-294), the 19S CaMV (see also U.S. Pat. No. 5,352,605 and WO 84/02913) or plant promoters like those from Rubisco small subunit described in U.S. Pat. No. 4,962,028. Even more preferred are seed-specific promoters driving expression of LMP proteins during all or selected stages of seed development. Seed-specific plant promoters are known to those of ordinary skill in the art and are identified and characterized using seed-specific mRNA libraries and expression profiling techniques. Seed-specific promoters include the napin-gene promoter from rapeseed (U.S. Pat. No. 5,608,152), the USP-promoter from Vicia faba (Baeumlein et al. 1991, Mol. Gen. Genetics 225:459-67), the oleosin-promoter from Arabidopsis (WO 98/45461), the phaseolin-promoter from Phaseolus vulgaris (U.S. Pat. No. 5,504,200), the Bce4-promoter from Brassica (WO9113980) or the legumin B4 promoter (LeB4; Baeumlein et al. 1992, Plant J. 2:233-239) as well as promoters conferring seed specific expression in monocot plants like maize, barley, wheat, rye, rice etc. Suitable promoters to note are the lpt2 or lpt1-gene promoter from barley (WO 95/15389 and WO 95/23230) or those described in WO 99/16890 (promoters from the barley hordein-gene, the rice glutelin gene, the rice oryzin gene, the rice prolamin gene, the wheat gliadin gene, wheat glutelin gene, the maize zein gene, the oat glutelin gene, the Sorghum kasirin-gene, and the rye secalin gene).

Plant gene expression can also be facilitated via an inducible promoter (for a review see Gatz 1997, Annu. Rev. Plant Physiol. Plant Mol. Biol. 48:89-108). Chemically inducible promoters are especially suitable if gene expression is desired in a time specific manner. Examples for such promoters are a salicylic acid inducible promoter (WO 95/19443), a tetracycline inducible promoter (Gatz et al. 1992, Plant J. 2:397-404) and an ethanol inducible promoter (WO 93/21334).

Promoters responding to biotic or abiotic stress conditions are also suitable promoters such as the pathogen inducible PRP1-gene promoter (Ward et al., 1993, Plant. Mol. Biol. 22:361-366), the heat inducible hsp80-promoter from tomato (U.S. Pat. No. 5,187,267), cold inducible alpha-amylase promoter from potato (WO 96/12814) or the wound-inducible pinil-promoter (EP 375091).

Other preferred sequences for use in plant gene expression cassettes are targeting-sequences necessary to direct the gene-product in its appropriate cell compartment (for review see Kermode 1996, Crit. Rev. Plant Sci. 15:285-423 and references cited therein) such as the vacuole, the nucleus, all types of plastids like amyloplasts, chloroplasts, chromoplasts, the extracellular space, mitochondria, the endoplasmic reticulum, oil bodies, peroxisomes and other compartments of plant cells. Also especially suited are promoters that confer plastid-specific gene expression, as plastids are the compartment where precursors and some end products of lipid biosynthesis are synthesized. Suitable promoters such as the viral RNA-polymerase promoter are described in WO 95/16783 and WO 97/06250 and the cipP-promoter from Arabidopsis described in WO 99/46394.

The invention further provides a recombinant expression vector comprising a DNA molecule of the invention cloned into the expression vector in an antisense orientation. That is, the DNA molecule is operatively linked to a regulatory sequence in a manner that allows for expression (by transcription of the DNA molecule) of an RNA molecule that is antisense to LMP mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al. (1986, Antisense RNA as a molecular tool for genetic analysis, Reviews—Trends in Genetics, Vol. 1) and Mol et al. (1990, FEBS Lett. 268:427-430).

Another aspect of the invention pertains to host cells into which a recombinant expression vector of the invention has been introduced. The terms “host cell” and “recombinant host cell” are used interchangeably herein. It is to be understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein. A host cell can be any prokaryotic or eukaryotic cell. For example, a LMP can be expressed in bacterial cells, insect cells, fungal cells, mammalian cells (such as Chinese hamster ovary cells (CHO) or COS cells), algae, ciliates or plant cells. Other suitable host cells are known to those skilled in the art.

Vector DNA can be introduced into prokaryotic or eukaryotic cells via conventional transformation or transfection techniques. As used herein, the terms “transformation” and “transfection”, “conjugation” and “transduction” are intended to refer to a variety of art-recognized techniques for introducing foreign nucleic acid (e.g., DNA) into a host cell, including calcium phosphate or calcium chloride coprecipitation, DEAE-dextran-mediated transfection, lipofection, natural competence, chemical-mediated transfer, or electroporation. Suitable methods for transforming or transfecting host cells including plant cells can be found in Sambrook et al. (1989, Molecular Cloning: A Laboratory Manual. 2nd, ed, Cold Spring Harbor Laboratory, Cold Spring Harbor Laboratory Press, Cold Spring Harbor, N.Y.) and other laboratory manuals such as Methods in Molecular Biology 1995, Vol. 44, Agrobacterium protocols, ed: Gartland and Davey, Humana Press, Totowa, N.J.

For stable transfection of mammalian and plant cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those that confer resistance to drugs, such as G418, hygromycin, kanamycin and methotrexate or in plants that confer resistance towards an herbicide such as glyphosate or glufosinate. A nucleic acid encoding a selectable marker can be introduced into a host cell on the same vector as that encoding a LMP or can be introduced on a separate vector. Cells stably transfected with the introduced nucleic acid can be identified by, for example, drug selection (e.g., cells that have incorporated the selectable marker gene will survive, while the other cells die).

To create a homologous recombinant microorganism, a vector is prepared which contains at least a portion of a LMP gene into which a deletion, addition or substitution has been introduced to thereby alter, e.g., functionally disrupt, the LMP gene. Preferably, this LMP gene is an Brassica napus, Glycine max or Linum usitatissimum LMP gene, but it can be a homologue from a related plant or even from a mammalian, yeast, or insect source. In a preferred embodiment, the vector is designed such that, upon homologous recombination, the endogenous LMP gene is functionally disrupted (i.e., no longer encodes a functional protein; also referred to as a knock-out vector). Alternatively, the vector can be designed such that, upon homologous recombination, the endogenous LMP gene is mutated or otherwise altered but still encodes functional protein (e.g., the upstream regulatory region can be altered to thereby alter the expression of the endogenous LMP). To create a point mutation via homologous recombination, DNA-RNA hybrids can be used in a technique known as chimeraplasty (Cole-Strauss et al. 1999, Nucleic Acids Res. 27:1323-1330 and Kmiec 1999, American Scientist 87:240-247). Homologous recombination procedures in Arabidopsis thaliana or other crops are also well known in the art and are contemplated for use herein.

In a homologous recombination vector, the altered portion of the LMP gene is flanked at its 5′ and 3′ ends by additional nucleic acid of the LMP gene to allow for homologous recombination to occur between the exogenous LMP gene carried by the vector and an endogenous LMP gene in a microorganism or plant. The additional flanking LMP nucleic acid is of sufficient length for successful homologous recombination with the endogenous gene. Typically, several hundreds of base pairs up to kilobases of flanking DNA (both at the 5′ and 3′ ends) are included in the vector (see e.g., Thomas & Capecchi 1987, Cell 51:503, for a description of homologous recombination vectors). The vector is introduced into a microorganism or plant cell (e.g., via polyethyleneglycol mediated DNA). Cells in which the introduced LMP gene has homologously recombined with the endogenous LMP gene are selected using art-known techniques.

In another embodiment, recombinant microorganisms can be produced which contain selected systems, which allow for regulated expression of the introduced gene. For example, inclusion of a LMP gene on a vector placing it under control of the lac operon permits expression of the LMP gene only in the presence of IPTG. Such regulatory systems are well known in the art.

A host cell of the invention, such as a prokaryotic or eukaryotic host cell in culture can be used to produce (i.e., express) a LMP. Accordingly, the invention further provides methods for producing LMPs using the host cells of the invention. In one embodiment, the method comprises culturing a host cell of the invention (into which a recombinant expression vector encoding a LMP has been introduced, or which contains a wild-type or altered LMP gene in it's genome) in a suitable medium until LMP is produced. In another embodiment, the method further comprises isolating LMPs from the medium or the host cell.

Another aspect of the invention pertains to isolated LMPs, and biologically active portions thereof. An “isolated” or “purified” protein or biologically active portion thereof is substantially free of cellular material when produced by recombinant DNA techniques, or chemical precursors or other chemicals when chemically synthesized. The language “substantially free of cellular material” includes preparations of LMP in which the protein is separated from cellular components of the cells in which it is naturally or recombinantly produced. In one embodiment, the language “substantially free of cellular material” includes preparations of LMP having less than about 30% (by dry weight) of non-LMP (also referred to herein as a “contaminating protein”), more preferably less than about 20% of non-LMP, still more preferably less than about 10% of non-LMP, and most preferably less than about 5% non-LMP. When the LMP or biologically active portion thereof is recombinantly produced, it is also preferably substantially free of culture medium, i.e., culture medium represents less than about 20%, more preferably less than about 10%, and most preferably less than about 5% of the volume of the protein preparation. The language “substantially free of chemical precursors or other chemicals” includes preparations of LMP in which the protein is separated from chemical precursors or other chemicals that are involved in the synthesis of the protein. In one embodiment, the language “substantially free of chemical precursors or other chemicals” includes preparations of LMP having less than about 30% (by dry weight) of chemical precursors or non-LMP chemicals, more preferably less than about 20% chemical precursors or non-LMP chemicals, still more preferably less than about 10% chemical precursors or non-LMP chemicals, and most preferably less than about 5% chemical precursors or non-LMP chemicals. In preferred embodiments, isolated proteins or biologically active portions thereof lack contaminating proteins from the same organism from which the LMP is derived. Typically, such proteins are produced by recombinant expression of, for example, an Brassica napus, Glycine max or Linum usitatissimum LMP in other plants than Brassica napus, Glycine max or Linum usitatissimum or microorganisms, algae or fungi.

An isolated LMP or a portion thereof of the invention can participate in the metabolism of compounds necessary for the production of seed storage compounds in Brassica napus, Glycine max or Linum usitatissimum or of cellular membranes, or has one or more of the activities set forth in Table 3. In preferred embodiments, the protein or portion thereof comprises an amino acid sequence which is sufficiently homologous to an amino acid sequence encoded by a nucleic acid of Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23, such that the protein or portion thereof maintains the ability to participate in the metabolism of compounds necessary for the construction of cellular membranes in Brassica napus, Glycine max or Linum usitatissimum, or in the transport of molecules across these membranes. The portion of the protein is preferably a biologically active portion as described herein. In another preferred embodiment, a LMP of the invention has an amino acid sequence encoded by a nucleic acid of Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23. In yet another preferred embodiment, the LMP has an amino acid sequence which is encoded by a nucleotide sequence which hybridizes, e.g., hybridizes under stringent conditions, to a nucleotide sequence of Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23. In still another preferred embodiment, the LMP has an amino acid sequence which is encoded by a nucleotide sequence that is at least about 50-60%, preferably at least about 60-70%, more preferably at least about 70-80%, 80-90%, 90-95% or more, that means preferably 70%, 72.5%, 75%, 77.5%, 80%, 82.5%, 85%, 87.5%, 90%, 92.5% or 95% or more and even more preferably at least about 96%, 97%, 98%, 99% or more homologous to one of the amino acid sequences encoded by a nucleic acid of Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23. The preferred LMPs of the present invention also preferably possess at least one of the LMP activities described herein. For example, a preferred LMP of the present invention includes an amino acid sequence encoded by a nucleotide sequence which hybridizes, e.g., hybridizes under stringent conditions, to a nucleotide sequence of Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23, and which can participate in the metabolism of compounds necessary for the construction of cellular membranes in Brassica napus, Glycine max or Linum usitatissimum, or in the transport of molecules across these membranes, or which has one or more of the activities set forth in Table 3.

In other embodiments, the LMP is substantially homologous to an amino acid sequence encoded by a nucleic acid of Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23, and retains the functional activity of the protein of one of the sequences encoded by a nucleic acid of Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23, yet differs in amino acid sequence due to natural variation or mutagenesis, as described in detail above. Accordingly, in another embodiment, the LMP is a protein which comprises an amino acid sequence which is at least about 50-60%, preferably at least about 60-70%, and more preferably at least about 70-80, 80-90, 90-95%, that means preferably 70%, 72.5%, 75%, 77.5%, 80%, 82.5%, 85%, 87.5%, 90%, 92.5% or 95% or more and most preferably at least about 96%, 97%, 98%, 99% or more homologous to an entire amino acid sequence and which has at least one of the LMP activities described herein. In another embodiment, the inventon pertains to a full Brassica napus, Glycine max or Linum usitatissimum protein which is substantially homologous to an entire amino acid sequence encoded by a nucleic acid of Appendix A, in a preferred embodiment as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23.

Dominant negative mutations or trans-dominant suppression can be used to reduce the activity of a LMP in transgenics seeds in order to change the levels of seed storage compounds. To achieve this a mutation that abolishes the activity of the LMP is created and the inactive non-functional LMP gene is overexpressed in the transgenic plant. The inactive trans-dominant LMP protein competes with the active endogenous LMP protein for substrate or interactions with other proteins and dilutes out the activity of the active LMP. In this way the biological activity of the LMP is reduced without actually modifying the expression of the endogenous LMP gene. This strategy was used by Pontier et al to modulate the activity of plant transcription factors (Pontier D, Miao Z H, Lam E, Plant J 2001 Sep. 27(6): 529-38, Trans-dominant suppression of plant TGA factors reveals their negative and posibve roles in plant defense responses).

Homologues of the LMP can be generated by mutagenesis, e.g., discrete point mutation or truncation of the LMP. As used herein, the term “homologue” refers to a variant form of the LMP that acts as an agonist or antagonist of the activity of the LMP. An agonist of the LMP can retain substantially the same, or a subset, of the biological activities of the LMP. An antagonist of the LMP can inhibit one or more of the activities of the naturally occurring form of the LMP, by, for example, competitively binding to a downstream or upstream member of the cell membrane component metabolic cascade which includes the LMP, or by binding to a LMP which mediates transport of compounds across such membranes, thereby preventing translocation from taking place.

In an alternative embodiment, homologues of the LMP can be identified by screening combinatorial libraries of mutants, e.g., truncation mutants, of the LMP for LMP agonist or antagonist activity. In one embodiment, a variegated library of LMP variants is generated by combinatorial mutagenesis at the nucleic acid level and is encoded by a variegated gene library. A variegated library of LMP variants can be produced by, for example, enzymatically ligating a mixture of synthetic oligonucleotides into gene sequences such that a degenerate set of potential LMP sequences is expressible as individual polypeptides, or alternatively, as a set of larger fusion proteins (e.g., for phage display) containing the set of LMP sequences therein. There are a variety of methods that can be used to produce libraries of potential LMP homologues from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential LMP sequences. Methods for synthesizing degenerate oligonucleotides are known in the art (see, e.g., Narang 1983, Tetrahedron 39:3; Itakura et al. 1984, Annu. Rev. Biochem. 53:323; Itakura et al. 1984, Science 198:1056; Ike et al. 1983, Nucleic Acids Res. 11:477).

In addition, libraries of fragments of the LMP coding sequences can be used to generate a variegated population of LMP fragments for screening and subsequent selection of homologues of a LMP. In one embodiment, a library of coding sequence fragments can be generated by treating a double stranded PCR fragment of a LMP coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the LMP.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of LMP homologues. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify LMP homologues (Arkin & Yourvan 1992, Proc. Natl. Acad. Sci. USA 89:7811-7815; Delgrave et al. 1993, Protein Engineering 6:327-331).

In another embodiment, cell based assays can be exploited to analyze a variegated LMP library, using methods well known in the art.

The nucleic acid molecules, proteins, protein homologues, fusion proteins, primers, vectors, and host cells described herein can be used in one or more of the following methods: identification of Brassica napus, Glycine max or Linum usitatissimum and related organisms; mapping of genomes of organisms related to Brassica napus, Glycine max or Linum usitatissimum; identification and localization of Brassica napus, Glycine max or Linum usitatissimum sequences of interest; evolutionary studies; determination of LMP regions required for function; modulation of a LMP activity; modulation of the metabolism of one or more cell functions; modulation of the transmembrane transport of one or more compounds; and modulation of seed storage compound accumulation.

The plant Arabidopsis thaliana represents one member of higher (or seed) plants. It is related to other plants such as Brassica napus, Glycine max or Linum usitatissimum which require light to drive photosynthesis and growth. Plants like Brassica napus, Glycine max or Linum usitatissimum share a high degree of homology on the DNA sequence and polypeptide level, allowing the use of heterologous screening of DNA molecules with probes evolving from other plants or organisms, thus enabling the derivation of a consensus sequence suitable for heterologous screening or functional annotation and prediction of gene functions in third species. The ability to identify such functions can therefore have significant relevance, e.g., prediction of substrate specificity of enzymes. Further, these nucleic acid molecules may serve as reference points for the mapping of Arabidopsis genomes, or of genomes of related organisms.

The LMP nucleic acid molecules of the invention have a variety of uses. First, the nucleic acid and protein molecules of the invention may serve as markers for specific regions of the genome. This has utility not only in the mapping of the genome, but also for functional studies of Brassica napus, Glycine max or Linum usitatissimum proteins. For example, to identify the region of the genome to which a particular Brassica napus, Glycine max or Linum usitatissimum DNA-binding protein binds, the Brassica napus, Glycine max or Linum usitatissimum genome could be digested, and the fragments incubated with the DNA-binding protein. Those which bind the protein may be additionally probed with the nucleic acid molecules of the invention, preferably with readily detectable labels; binding of such a nucleic acid molecule to the genome fragment enables the localization of the fragment to the genome map of Brassica napus, Glycine max or Linum usitatissimum, and, when performed multiple times with different enzymes, facilitates a rapid determination of the nucleic acid sequence to which the protein binds. Further, the nucleic acid molecules of the invention may be sufficiently homologous to the sequences of related species such that these nucleic acid molecules may serve as markers for the construction of a genomic map in related plants.

The LMP nucleic acid molecules of the invention are also useful for evolutionary and protein structural studies. The metabolic and transport processes in which the molecules of the invention participate are utilized by a wide variety of prokaryotic and eukaryotic cells; by comparing the sequences of the nucleic acid molecules of the present invention to those encoding similar enzymes from other organisms, the evolutionary relatedness of the organisms can be assessed. Similarly, such a comparison permits an assessment of which regions of the sequence are conserved and which are not, which may aid in determining those regions of the protein which are essential for the functioning of the enzyme. This type of determination is of value for protein engineering studies and may give an indication of what the protein can tolerate in terms of mutagenesis without losing function.

Manipulation of the LMP nucleic acid molecules of the invention may result in the production of LMPs having functional differences from the wild-type LMPs. These proteins may be improved in efficiency or activity, may be present in greater numbers in the cell than is usual, or may be decreased in efficiency or activity.

There are a number of mechanisms by which the alteration of a LMP of the invention may directly affect the accumulation and/or composition of seed storage compounds. In the case of plants expressing LMPs, increased transport can lead to altered accumulation of compounds and/or solute partitioning within the plant tissue and organs which ultimately could be used to affect the accumulation of one or more seed storage compounds during seed development. An example is provided by Mitsukawa et al. (1997, Proc. Natl. Acad. Sci. USA 94:7098-7102), where overexpression of an Arabidopsis high-affinity phosphate transporter gene in tobacco cultured cells enhanced cell growth under phosphate-limited conditions. Phosphate availability also affects significantly the production of sugars and metabolic intermediates (Hurry et al. 2000, Plant J. 24:383-396) and the lipid composition in leaves and roots (Härtel et al. 2000, Proc. Natl. Acad. Sci. USA 97:10649-10654). Likewise, the activity of the plant ACCase has been demonstrated to be regulated by phosphorylation (Savage & Ohirogge 1999, Plant J. 18:521-527) and alterations in the activity of the kinases and phosphatases (LMPs) that act on the ACCase could lead to increased or decreased levels of seed lipid accumulation. Moreover, the presence of lipid kinase activities in chloroplast envelope membranes suggests that signal transduction pathways and/or membrane protein regulation occur in envelopes (see, e.g., MOller et al. 2000, J. Biol. Chem. 275:19475-19481 and literature cited therein). The ABI1 and ABI2 genes encode two protein serine/threonine phosphatases 2C, which are regulators in abscisic acid signaling pathway, and thereby in early and late seed development (e.g. Merlot et al. 2001, Plant J. 25:295-303). For more examples see also the section ‘background of the invention’.

The present invention also provides antibodies that specifically bind to an LMP-polypeptide, or a portion thereof, as encoded by a nucleic acid disclosed herein or as described herein.

Antibodies can be made by many well-known methods (see, e.g. Harlow and Lane, “Antibodies; A Laboratory Manual” Cold Spring Harbor Laboratory, Cold Spring Harbor, N.Y., 1988). Briefly, purified antigen can be injected into an animal in an amount and in intervals sufficient to elicit an immune response. Antibodies can either be purified directly, or spleen cells can be obtained from the animal. The cells can then fused with an immortal cell line and screened for antibody secretion. The antibodies can be used to screen nucleic acid clone libraries for cells secreting the antigen. Those positive clones can then be sequenced (see, for example, Kelly et al. 1992, Bio/Technology 10:163-167; Bebbington et al. 1992, Bio/Technology 10:169-175).

The phrase “selectively binds” with the polypeptide refers to a binding reaction which is determinative of the presence of the protein in a heterogeneous population of proteins and other biologics. Thus, under designated immunoassay conditions, the specified antibodies bound to a particular protein do not bind in a significant amount to other proteins present in the sample. Selective binding to an antibody under such conditions may require an antibody that is selected for its specificity for a particular protein. A variety of immunoassay formats may be used to select antibodies that selectively bind with a particular protein. For example, solid-phase ELISA immuno-assays are routinely used to select antibodies selectively immunoreactive with a protein. See Harlow and Lane “Antibodies, A Laboratory Manual” Cold Spring Harbor Publications, New York (1988), for a description of immunoassay formats and conditions that could be used to determine selective binding.

In some instances, it is desirable to prepare monoclonal antibodies from various hosts. A description of techniques for preparing such monoclonal antibodies may be found in Stites et al., editors, “Basic and Clinical Immunology,” (Lange Medical Publications, Los Altos, Calif., Fourth Edition) and references cited therein, and in Harlow and Lane (“Antibodies, A Laboratory Manual” Cold Spring Harbor Publications, New York, 1988).

Throughout this application, various publications are referenced. The disclosures of all of these publications and those references cited within those publications in their entireties are hereby incorporated by reference into this application in order to more fully describe the state of the art to which this invention pertains.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and Examples be considered as exemplary only, with a true scope and spirit of the invention being indicated by the claims included herein.

### EXAMPLES

### Example 1

### General Processes

a) General Cloning Processes:

Cloning processes such as, for example, restriction cleavages, agarose gel electrophoresis, purification of DNA fragments, transfer of nucleic acids to nitrocellulose and nylon membranes, linkage of DNA fragments, transformation of Escherichia coli and yeast cells, growth of bacteria and sequence analysis of recombinant DNA were carried out as described in Sambrook et al. (1989, Cold Spring Harbor Laboratory Press: ISBN 0-87969-309-6) or Kaiser, Michaelis and Mitchell (1994, “Methods in Yeast Genetics”, Cold Spring Harbor Laboratory Press: ISBN 087969-451-3).

b) Chemicals:

The chemicals used were obtained, if not mentioned otherwise in the text, in p.a. quality from the companies Fluka (Neu-Ulm), Merck (Darmstadt), Roth (Karlsruhe), Serva (Heidelberg) and Sigma (Deisenhofen). Solutions were prepared using purified, pyrogen-free water, designated as H2O in the following text, from a Milli-Q water system water purification plant (Millipore, Eschbom). Restriction endonucleases, DNA-modifying enzymes and molecular biology kits were obtained from the companies AGS (Heidelberg), Amersham (Braunschweig), Biometra (Gattingen), Roche (Mannheim), Genomed (Bad Oeynnhausen), New England Biolabs (Schwalbach/Taunus), Novagen (Madison, Wis., USA), Perkin-Elmer (Weiterstadt), Pharmacia (Freiburg), Qiagen (Hilden) and Stratagene (Amsterdam, Netherlands). They were used, if not mentioned otherwise, according to the manufacturer's instructions.

c) Plant Material and Growth: Arabidopsis Plants

For this study, root material, leaves, siliques and seeds of wild-type and transgenic plants of Arabidopsis thaliana expressing KCS-like proteins were used. Wild type and KCS Arabidopsis seeds were preincubated for three days in the dark at 4° C. before placing them into an incubator (AR-75, Percival Scientific, Boone, Iowa) at a photon flux density of 60-80 μmol m⁻² s⁻¹ and a light period of 16 hours (22° C.), and a dark period of 8 hours (18° C.). All plants were started on half-strength MS medium (Murashige & Skoog, 1962, Physiol. Plant. 15, 473-497), pH 6.2, 2% sucrose and 1.2% agar. Seeds were sterilized for 20 minutes in 20% bleach 0.5% triton X100 and rinsed 6 times with excess sterile water. Brassica napus

Brassica napus varieties AC Excel and Cresor were used for this study to create cDNA libraries. Seed, seed pod, flower, leaf, stem and root tissues were collected from plants that were in some cases dark-, salt-, heat- and drought-treated. However, this study focused on the use of seed and seed pod tissues for cDNA libraries. Plants were tagged to harvest seeds collected 60-75 days after planting from two time points: 1-15 days and 15-25 days after anthesis. Plants have been grown in Metromix (Scotts, Marysville, Ohio) at 71° F. under a 14 hr photoperiod. Six seed and seed pod tissues of interest in this study were collected to create the following cDNA libraries: Immature seeds, mature seeds, immature seed pods, mature seed pods, night-harvested seed pods and Cresor variety (high erucic acid) seeds. Tissue samples were collected within specified time points for each developing tissue and multiple samples within a time frame pooled together for eventual extraction of total RNA. Samples from immature seeds were taken between 1-25 days after anthesis (daa), mature seeds between 25-50 daa, immature seed pods between 1-15 daa, mature seed pods between 15-50 daa, night-harvested seed pods between 1-50 daa and Cresor seeds 5-25 daa.

### Glycine max

Glycine max cv. Resnick was used for this study to create cDNA libraries. Seed, seed pod, flower, leaf, stem and root tissues were collected from plants that were in some cases dark-, salt-, heat- and drought-treated. In some cases plants have been nematode infected as well. However, this study focused on the use of seed and seed pod tissues for cDNA libraries. Plants were tagged to harvest seeds at the set days after anthesis: 5-15, 15-25, 25-35, & 33-50.

### Oryza sativa

Oryza sativa ssp. Japonica cv. Nippon-barre was used for this study to create cDNA libraries. Seed, seed pod, flower, leaf, stem and root tissues were collected from plants that were in some cases dark-, salt-, heat- and drought-treated. This study focused on the use of seed embryo tissues for cDNA libraries. Embryo and endosperm were collected separately in case endosperm tissue might interfere with RNA extraction. Plants have been grown in the greenhouse on Wisconsin soil (has high organic matter) at 85° F. under a 14-h photoperiod. Rice embryos were dissected out of the developing seeds.

### Triticum aestivum

Triticum aestivum cv. Galeon was used for this study to create cDNA libraries. Seed, flower, fruits, leaf, stem and root tissues were collected from plants that were in some cases dark-, salt-, heat- and drought-treated. Plants have been grown in the greenhouse in metromix under a 12-h photoperiod at 72° F. during the day period and 65° F. during the night period.

### Example 2

### Total DNA Isolation from Plants

The details for the isolation of total DNA relate to the working up of one gram fresh weight of plant material.

CTAB buffer: 2% (w/v) N-cethyl-N,N,N-trimethylammonium bromide (CTAB); 100 mM Tris HCl pH 8.0; 1.4 M NaCl; 20 mM EDTA. N-Laurylsarcosine buffer: 10% (w/v) N-laurylsarcosine; 100 mM Tris HCl pH 8.0; 20 mM EDTA.

The plant material was triturated under liquid nitrogen in a mortar to give a fine powder and transferred to 2 ml Eppendorf vessels. The frozen plant material was then covered with a layer of 1 ml of decomposition buffer (1 ml CTAB buffer, 100 μl of N-laurylsarcosine buffer, 20 μl of β-mercaptoethanol and 10 μl of proteinase K solution, 10 mg/ml) and incubated at 60° C. for one hour with continuous shaking. The homogenate obtained was distributed into two Eppendorf vessels (2 ml) and extracted twice by shaking with the same volume of chloroform/isoamyl alcohol (24:1). For phase separation, centrifugation was carried out at 8000 g and RT for 15 min in each case. The DNA was then precipitated at −70° C. for 30 min using ice-cold isopropanol. The precipitated DNA was sedimented at 4° C. and 10,000 g for 30 min and resuspended in 180 μl of TE buffer (Sambrook et al. 1989, Cold Spring Harbor Laboratory Press: ISBN 0-87969-3096). For further purification, the DNA was treated with NaCl (1.2 M final concentration) and precipitated again at −70° C. for 30 min using twice the volume of absolute ethanol. After a washing step with 70% ethanol, the DNA was dried and subsequently taken up in 50 μl of H₂O+RNAse (50 mg/ml final concentration). The DNA was dissolved overnight at 4° C. and the RNAse digestion was subsequently carried out at 37° C. for 1 h. Storage of the DNA took place at 4° C.

### Example 3

### Isolation of Total RNA and poly-(A)+ RNA from Plants Arabidopsis thaliana

For the investigation of transcripts, both total RNA and poly-(A)+ RNA were isolated.

RNA is isolated from siliques of Arabidopsis plants according to the following procedure:RNA preparation from Arabidopsis seeds—“hot” extraction:

### 1. Buffers, Enzymes and Solution

2M KCl
Proteinase K
Phenol (for RNA)
Chloroform:Isoamylalcohol
(Phenol:choloroform 1:1; pH adjusted for RNA)
4 M LiCl, DEPC-treated
DEPC-treated water
3M NaOAc, pH 5, DEPC-treated
Isopropanol
70% ethanol (made up with DEPC-treated water)
Resuspension buffer:0.5% SDS, 10 mM Tris pH 7.5, 1 mM EDTA made up with

### DEPC—treated water as this solution can not be DEPC-treated

Extraction Buffer:
0.2M Na Borate
30 mM EDTA
30 mM EGTA
1% SDS (250 μl of 10% SDS-solution for 2.5 ml buffer)
1% Deoxycholate (25 mg for 2.5 ml buffer)
2% PVPP (insoluble—50 mg for 2.5 ml buffer)
2% PVP 40K (50 mg for 2.5 ml buffer)
10 mM DTT
   100 mM β-Mercaptoethanol (fresh, handle under fume hood—use 35 μl of 14.3M solution for 5 ml buffer)

### 2. Extraction

Heat extraction buffer up to 80° C. Grind tissue in liquid nitrogen-cooled mortar, transfer tissue powder to 1.5 ml tube. Tissue should kept frozen until buffer is added so transfer the sample with pre-cooled spatula and keep the tube in liquid nitrogen all time. Add 350 μl preheated extraction buffer (here for 100 mg tissue, buffer volume can be as much as 500 μl for bigger samples) to tube, vortex and heat tube to 80° C. for ˜1 min. Keep then on ice. Vortex sample, grind additionally with electric mortar.

### 3. Digestion

Add Proteinase K (0.15 mg/100 mg tissue), vortex and keep at 37° C. for one hour.

### First Purification

Add 27 μl 2M KCl. Chill on ice for 10 min. Centrifuge at 12.000 rpm for 10 minutes at room temperature. Transfer supernatant to fresh, RNAase-free tube and do one phenol extraction, followed by a chloroform:isoamylalcohol extraction. Add 1 vol. isopropanol to supernatant and chill on ice for 10 min. Pellet RNA by centrifugation (7000 rpm for 10 min at RT). Resolve pellet in 1 ml 4M LiCl by 10 to 15 min vortexing. Pellet RNA by 5 min centrifugation.

### Second Purification

Resuspend pellet in 500 μl Resuspension buffer. Add 500 μl phenol and vortex. Add 250 μl chloroform:isoamylalcohol and vortex. Spin for 5 min. and transfer supernatant to fresh tube. Repeat chloform:isoamylalcohol extraction until interface is clear. Transfer supernatant to fresh tube and add 1/10 vol 3M NaOAc, pH 5 and 600 μl isopropanol. Keep at −20 for 20 min or longer. Pellet RNA by 10 min centrifugation. Wash pellet once with 70% ethanol. Remove all remaining alcohol before resolving pellet with 15 to 20 μl DEPC-water. Determine quantity and quality by measuring the absorbance of a 1:200 dilution at 260 and 280 nm. 40 μg RNA/ml=1OD260

RNA from wild-type and the transgenic Arabidopsis-plants is isolated as described (Hosein, 2001, Plant Mol Biol Rep., 19, 65a-65e; Ruuska, S. A., Girke, T., Benning, C., & Ohlrogge, J. B., 2002, Plant Cell, 14, 1191-1206).

The mRNA is prepared from total RNA, using the Amersham Pharmacia Biotech mRNA purification kit, which utilizes oligo(dT)-cellulose columns.

Isolation of Poly-(A)+ RNA was isolated using Dyna BeadsR (Dynal, Oslo, Norway) following the instructions of the manufacturer's protocol. After determination of the concentration of the RNA or of the poly(A)+ RNA, the RNA was precipitated by addition of 1/10 volumes of 3 M sodium acetate pH 4.6 and 2 volumes of ethanol and stored at −70° C.

### Brassica napus, Glydne max and Linum usitatissimum

Brassica napus, Linum usitatissimum and Glycine max seeds were separated from pods to create homogeneous materials for seed and seed pod cDNA libraries. Tissues were ground into fine powder under liquid N₂ using a mortar and pestle and transferred to a 50 ml tube. Tissue samples were stored at 80° C. until extractions could be performed.

Total RNA was extracted from tissues using RNeasy Maxi kit (Qiagen) according to manufacture's protocol and mRNA was processed from total RNA using Oligotex mRNA Purification System kit (Qiagen), also according to manufacture's protocol. mRNA was sent to Hyseq Pharmaceuticals Incorporated (Sunnyville, Calif.) for further processing of mRNA from each tissue type into cDNA libraries and for use in their proprietary processes in which similar inserts in plasmids are clustered based on hybridization patterns.

### Example 4

### cDNA Library Construction

For cDNA library construction, first strand synthesis was achieved using Murine Leukemia Virus reverse transcriptase (Roche, Mannheim, Germany) and oligo-d(T)-primers, second strand synthesis by incubation with DNA polymerase 1, Klenow enzyme and RNAseH digestion at 12° C. (2 h), 16° C. (1 h) and 22° C. (1 h). The reaction was stopped by incubation at 65° C. (10 min) and subsequently transferred to ice. Double stranded DNA molecules were blunted by T4-DNApolymerase (Roche, Mannheim) at 37° C. (30 min). Nucleotides were removed by phenol/chloroform extraction and Sephadex G50 spin columns. EcoRI adapters (Pharmacia, Freiburg, Germany) were ligated to the cDNA ends by T4-DNA-ligase (Roche, 12° C., overnight) and phosphorylated by incubation with polynucleotide kinase (Roche, 37° C., 30 min). This mixture was subjected to separation on a low melting agarose gel. DNA molecules larger than 300 base pairs were eluted from the gel, phenol extracted, concentrated on Elutip-D-columns (Schleicher and Schuell, Dassel, Germany) and were ligated to vector arms and packed into lambda ZAPII phages or lambda ZAP-Express phages using the Gigapack Gold Kit (Stratagene, Amsterdam, Netherlands) using material and following the instructions of the manufacturer.

Brassica napus, Glycine max and Linum usitatissimum cDNA Libraries were generated at Hyseq Pharmaceuticals Incorporated (Sunnyville, Calif.) No amplification steps were used in the library production to retain expression information. Hyseq's genomic approach involves grouping the genes into clusters and then sequencing representative members from each cluster. cDNA libraries were generated from oligo dT column purified mRNA. Colonies from transformation of the cDNA library into E. coli were randomly picked and the cDNA insert were amplified by PCR and spotted on nylon membranes. A set of ³³-P radiolabeled oligonucleotides were hybridized to the clones and the resulting hybridization pattern determined to which cluster a particular clone belonged. cDNA clones and their DNA sequences were obtained for use in overexpression in transgenic plants and in other molecular biology processes described herein.

### Example 5

### Identification of LMP Genes of Interest that Are KCS-Like KCS of Arabidopsis thaliana

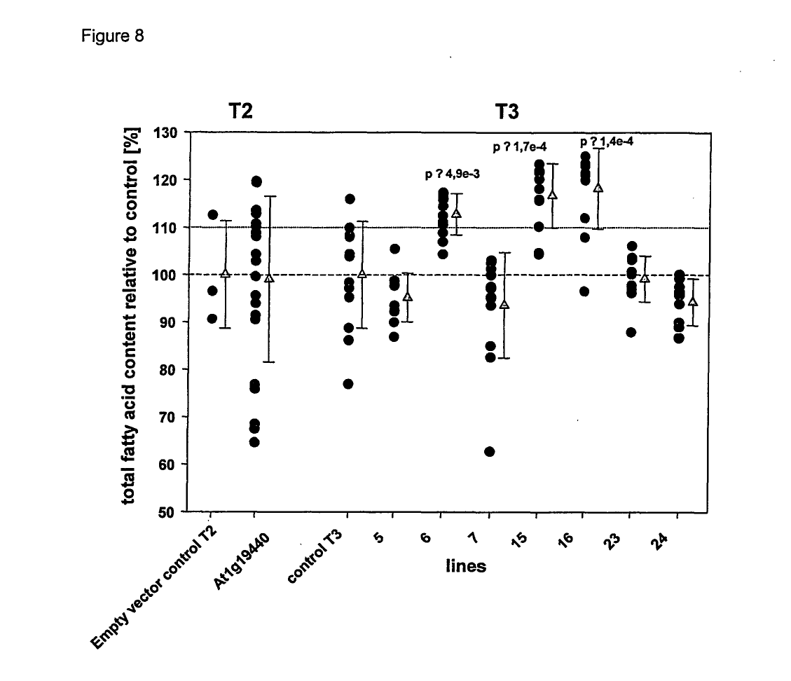

The Arabidopsis gene At1g19440, coding for a putative beta-ketoacyl-CoA synthase was used to identify LMP-encoding genes. It has been shown that overexpression of At1g19440 resulted in an increase in the oil content in transgenic Arabidopsis seeds (FIG. 8; WO2004013304-A2)

### Brassica napus, Glyaine max and Linum usitatissimum

This example illustrates how cDNA clones encoding KCS-like polypeptides of Brassica napus, Glycine max and Linum usitatissimum were identified and isolated.

In order to identify KCS-like genes in propriety databases, a similarity analysis using BLAST software (Basic Local Alignment Search Tool, Altschul et al., 1990, J. Mol. Biol. 215:403-410) was carry out. The amino acid sequence of the Arabidopsis KCS (At1g19940) polypeptide was used as a query to search and align DNA databases from Brassica napus, Glycine max and Linum usitatissimum that were translated in all six reading frames, using the TBLASTN algorithm. Such similarity analysis of the BPS in-house databases resulted in the identification of numerous ESTs and cDNA contigs.

RNA expression profile data obtained from the Hyseq clustering process were used to determine organ-specificity. Clones showing a greater expression in seed libraries compared to the other tissue libraries were selected as LMP candidate genes. The Brassica napus, Glycine max and Linum usitatissimum clones were selected for overexpression in Arabidopsis based on their expression profile.

### Example 6

### Cloning of Full-Length cDNAs and Orthologs of Identified LMP Genes

Clones corresponding to full-length sequences and partial cDNAs from Brassica napus, Glycine max or Linum usitatissimum had been identified in the in-house proprietary Hyseq databases. The Hyseq clones of Brassica napus, Glycine max and Linum usitatissimum genes were sequenced at DNA Landmarks using a ABI 377 slab gel sequencer and BigDye Terminator Ready Reaction kits (PE Biosystems, Foster City, Calif.). Sequence algingments were done to determine whether the Hyseq clones were full-length or partial clones. In cases where the Hyseq clones were determined to be partial cDNAs the following procedure was used to isolate the full-length sequences. Full-length cDNAs were isolated by RACE PCR using the SMART RACE cDNA amplification kit from Clontech allowing both 5′- and 3′ rapid amplification of cDNA ends (RACE). The RACE PCR primers were designed based on the Hyseq clone sequences. The isolation of full-length cDNAs and the RACE PCR protocol used were based on the manufacturer's conditions. The RACE product fragments were extracted from agarose gels with a QIAquick Gel Extraction Kit (Qiagen) and ligated into the TOPO pCR 2.1 vector (Invitrogen) following manufacturer's instructions. Recombinant vectors were transformed into TOP10 cells (Invitrogen) using standard conditions (Sambrook et al. 1989). Trans-formed cells were grown overnight at 37° C. on LB agar containing 50 μg/ml kanamycin and spread with 40 μl of a 40 mg/ml stock solution of X-gal in dimethylformamide for blue-white selection. Single white colonies were selected and used to inoculate 3 ml of liquid LB containing 50 μg/ml kanamycin and grown overnight at 37° C. Plasmid DNA is extracted using the QIAprep Spin Miniprep Kit (Qiagen) following manufacturer's instructions. Subsequent analyses of clones and restriction mapping were performed according to standard molecular biology techniques (Sambrook et al. 1989).

Full-length cDNAs were isolated and cloned into biary vectors by using the following procedure: Gene specific primers were designed using the full-length sequences obtained from Hyseq clones or subsequent RACE amplification products. Full-length sequences and genes were amplified utilizing Hyseq clones or cDNA libraries as DNA template using touchdown PCR. In some cases, primers were designed to add an “AACA” Kozak-like sequence just upstream of the gene start codon and two bases downstream were, in some cases, changed to GC to facilitate increased gene expression levels (Chandrashekhar et al. 1997, Plant Molecular Biology 35:993-1001). PCR reaction cycles were: 94° C., 5 min; 9 cycles of 94° C., 1 min, 65° C., 1 min, 72° C., 4 min and in which the anneal temperature was lowered by 1° C. each cycle; 20 cycles of 94° C., 1 min, 55° C., 1 min, 72° C., 4 min; and the PCR cycle was ended with 72° C., 10 min. Amplified PCR products were gel purified from 1% agarose gels using GenElute—EtBr spin columns (Sigma) and after standard enzymatic digestion, were ligated into the plant binary vector PBPSGB1 for transformation of Arabidopsis. The binary vector was amplified by overnight growth in E. coli DH5 in LB media and appropriate antibiotic and plasmid was prepared for downstream steps using Qiagen MiniPrep DNA preparation kit. The insert was verified throughout the various cloning steps by determining its size through restriction digest and inserts were sequenced to ensure the expected gene was used in Arabidopsis transformation.

Gene sequences can be used to identify homologous or heterologous genes (orthologs, the same LMP gene from another plant) from cDNA or genomic libraries. This can be done by designing PCR primers to conserved sequences identified by multiple sequence alignments. Orthologs are often identified by designing degenerate primers to full-length or partial sequences of genes of interest.

Gene sequences can be used to identify homologues or orthologs from cDNA or genomic libraries. Homologous genes (e.g. full-length cDNA clones) can be isolated via nucleic acid hybridization using for example cDNA libraries: Depending on the abundance of the gene of interest, 100,000 up to 1,000,000 recombinant bacteriophages are plated and transferred to nylon membranes. After denaturation with alkali, DNA is immobilized on the membrane by e.g. UV cross-linking. Hybridization is carried out at high stringency conditions. Aqueous solution hybridization and washing is performed at an ionic strength of 1 M NaCl and a temperature of 68° C. Hybridization probes are generated by e.g. radioactive (32P) nick transcription labeling (High Prime, Roche, Mannheim, Germany). Signals are detected by autoradiography.

Partially homologous or heterologous genes that are related but not identical can be identified in a procedure analogous to the above-described procedure using low stringency hybridization and washing conditions. For aqueous hybridization, the ionic strength is normally kept at 1 M NaCl while the temperature is progressively lowered from 68 to 42° C.

Isolation of gene sequences with homologies (or sequence identity/similarity) only in a distinct domain of (for example 10-20 amino acids) can be carried out by using synthetic radio labeled oligonucleotide probes. Radio labeled oligonucleotides are prepared by phosphorylation of the 5-prime end of two complementary oligonucleotides with T4 polynucleotide kinase. The complementary oligonucleotides are annealed and ligated to form concatemers. The double stranded concatemers are than radiolabeled by for example nick transcription. Hybridization is normally performed at low stringency conditions using high oligonucleotide concentrations.

Oligonucleotide hybridization solution:
6×SSC
b) M sodium phosphate
mM EDTA (pH 8)
0.5% SDS
100 μg/ml denaturated salmon sperm DNA
c) % nonfat dried milk

During hybridization, temperature is lowered stepwise to 5-10° C. below the estimated oligonucleotide Tm or down to room temperature followed by washing steps and autoradiography. Washing is performed with low stringency such as 3 washing steps using 4×SSC. Further details are described by Sambrook et al. (1989, “Molecular Cloning: A Laboratory Manual”, Cold Spring Harbor Laboratory Press) or Ausubel et al. (1994, “Current Protocols in Molecular Biology”, John Wiley & Sons).

### Example 7

### Identification of Genes of Interest by Screening Expression Libraries with Antibodies

cDNA clones can be used to produce recombinant protein for example in E. coli(e.g. Qiagen QIAexpress pQE system). Recombinant proteins are then normally affinity purified via Ni-NTA affinity chromatography (Qiagen). Recombinant proteins can be used to produce specific antibodies for example by using standard techniques for rabbit immunization. Antibodies are affinity purified using a Ni-NTA column saturated with the recombinant antigen as described by Gu et al. (1994, BioTechniques 17:257-262). The antibody can then be used to screen expression cDNA libraries to identify homologous or heterologous genes via an immunological screening (Sambrook et al. 1989, Molecular Cloning: A Laboratory Manual”, Cold Spring Harbor Laboratory Press or Ausubel et al. 1994, “Current Protocols in Molecular Biology”, John Wiley & Sons).

### Example 8

### Northern-Hybridization

For RNA hybridization, 20 μg of total RNA or 1 μg of poly-(A)+ RNA is separated by gel electrophoresis in 1.25% agarose gels using formaldehyde as described in Amasino (1986, Anal. Biochem. 152:304), transferred by capillary attraction using 10×SSC to positively charged nylon membranes (Hybond N+, Amersham, Braunschweig), immobilized by UV light and pre-hybridized for 3 hours at 68° C. using hybridization buffer (10% dextran sulfate w/v, 1 M NaCl, 1% SDS, 100 μg/ml of herring sperm DNA). The labeling of the DNA probe with the Highprime DNA labeling kit (Roche, Mannheim, Germany) is carried out during the pre-hybridization using alpha-32P dCTP (Amersham, Braunschweig, Germany). Hybridization is carried out after addition of the labeled DNA probe in the same buffer at 68° C. overnight. The washing steps are carried out twice for 15 min using 2×SSC and twice for 30 min using 1×SSC, 1% SDS at 68° C. The exposure of the sealed filters is carried out at −70° C. for a period of 1 day to 14 days.

### Example 9

### DNA Sequencing and Computational Functional Analysis

cDNA-libraries can be used for DNA sequencing according to standard methods, in particular by the chain termination method using the ABI PRISM Big Dye Terminator Cycle Sequencing Ready Reaction Kit (Perkin-Elmer, Weiterstadt, Germany). Random sequencing can be carried out subsequent to preparative plasmid recovery from cDNA libraries via in vivo mass excision, retransformation, and subsequent plating of DH10B on agar plates (material and protocol details from Stratagene, Amsterdam, Netherlands). Plasmid DNA can be prepared from overnight grown E. coli cultures grown in Luria-Broth medium containing ampicillin (see Sambrook et al. (1989, Cold Spring Harbor Laboratory Press: ISBN 0-87969-309-6) on a Qiagene DNA preparation robot (Qiagen, Hilden) according to the manufacturer's protocols). Sequences can be processed and annotated using the software package EST-MAX commercially provided by Bio-Max (Munich, Germany). The program incorporates bioinformatics methods important for functional and structural characterization of protein sequences. For reference see http://pedant.mips.biochem.mpg.de.

The most important algorithms incorporated in EST-MAX are: FASTA: Very sensitive protein sequence database searches with estimates of statistical significance (Pearson W. R. 1990, Rapid and sensitive sequence comparison with FASTP and FASTA. Methods Enzymol. 183:63-98). BLAST: Very sensitive protein sequence database searches with estimates of statistical significance (Altschul S. F., Gish W., Miller W., Myers E. W. and Lipman D. J. Basic local alignment search tool. J. Mol. Biol. 215:403-410). PREDATOR: High-accuracy secondary structure prediction from single and multiple sequences. (Frishman & Argos 1997, 75% accuracy in protein secondary structure prediction. Proteins 27:329-335). CLUSTALW: Multiple sequence alignment (Thompson, J. D., Higgins, D. G. and Gibson, T. J. 1994, CLUSTAL W: improving the sensitivity of progressive multiple sequence alignment through sequence weighting, positions-specific gap penalties and weight matrix choice, Nucleic Acids Res. 22:4673-4680). TMAP: Transmembrane region prediction from multiply aligned sequences (Persson B. & Argos P. 1994, Prediction of transmembrane segments in proteins utilizing multiple sequence alignments, J. Mol. Biol. 237:182-192). ALOM2:Transmembrane region prediction from single sequences (Klein P., Kanehisa M., and DeLisi C. 1984, Prediction of protein function from sequence properties: A discriminant analysis of a database. Biochim. Biophys. Acta 787:221-226. Version 2 by Dr. K. Nakai). PROSEARCH: Detection of PROSITE protein sequence patterns. Kolakowski L. F. Jr., Leunissen J. A. M. and Smith J. E. 1992, ProSearch: fast searching of protein sequences with regular expression patterns related to protein structure and function. Biotechniques 13:919-921). BLIMPS: Similarity searches against a database of ungapped blocks (Wallace & Henikoff 1992, PATMAT: A searching and extraction program for sequence, pattern and block queries and databases, CABIOS 8:249-254. Written by Bill Alford).

### Example 10

### Plasmids for Plant Transformation

For plant transformation binary vectors such as pBinAR can be used (Höfgen & Willmitzer 1990, Plant Sci. 66:221-230). Construction of the binary vectors can be performed by ligation of the cDNA in sense or antisense orientation into the T-DNA. 5-prime to the cDNA a plant promoter activates transcription of the cDNA. A polyadenylation sequence is located 3′-prime to the cDNA. Tissuespecific expression can be achieved by using a tissue specific promoter. For example, seed-specific expression can be achieved by cloning the napin or LeB4 or USP promoter 5-prime to the cDNA. Also any other seed specific promoter element can be used. For constitutive expression within the whole plant the CaMV 35S promoter can be used. The expressed protein can be targeted to a cellular compartment using a signal peptide, for example for plastids, mitochondria or endoplasmic reticulum (Kermode 1996, Crit. Rev. Plant Sci. 15:285-423). The signal peptide is cloned 5-prime in frame to the cDNA to achieve subcellular localization of the fusion protein.

Further examples for plant binary vectors are the pSUN300 or pSUN2-GW vectors into which the LMP gene candidates are cloned. These binary vectors contain an antibiotic resistance gene driven under the control of the NOS promoter and a USP seed-specific promoter (see FIG. 20) in front of the candidate gene with the NOSpA terminator or the OCS terminator. Partial or full-length LMP cDNA are cloned into the multiple cloning site of the plant binary vector in sense or antisense orientation behind the USP seed-specific promoters. The recombinant vector containing the gene of interest is transformed into Top10 cells (Invitrogen) using standard conditions. Transformed cells are selected for on LB agar containing 50 μg/ml kanamycin grown overnight at 37° C. Plasmid DNA is extracted using the QIAprep Spin Miniprep Kit (Qiagen) following manufacturer's instructions. Analysis of sub-sequent clones and restriction mapping is performed according to standard molecular biology techniques (Sambrook et al. 1989, Molecular Cloning, A Laboratory Manual. 2^{nd} Edition. Cold Spring Harbor Laboratory Press. Cold Spring Harbor, N.Y.).

### Example 11

### Agrobacterium Mediated Plant Transformation

Agrobacterium mediated plant transformation with the LMP nucleic acids described herein can be performed using standard transformation and regeneration techniques (Gelvin, Stanton B. & Schilperoort R. A, Plant Molecular Biology Manual, 2nd ed. Kluwer Academic Publ., Dordrecht 1995 in Sect., Ringbuc Zentrale Signatur:BT11-P; Glick, Bernard R. and Thompson, John E. Methods in Plant Molecular Biology and Biotechnology, S. 360, CRC Press, Boca Raton 1993). For example, Agrobacterium mediated transformation can be performed using the GV3 (pMP90) (Koncz & Schell, 1986, Mol. Gen. Genet. 204:383-396) or LBA4404 (Clontech) Agrobacterium tumefaiens strain.

Arabidopsis thaliana can be grown and transformed according to standard conditions (Bechtold 1993, Acad. Sci. Paris. 316:1194-1199; Bent et al. 1994, Science 265:1856-1860). Additionally, rapeseed can be transformed with the LMR nucleic acids of the present invention via cotyledon or hypocotyl transformation (Moloney et al. 1989, Plant Cell Report 8:238-242; De Block et al. 1989, Plant Physiol. 91:694-701). Use of antibiotic for Agrobacterium and plant selection depends on the binary vector and the Agrobacterium strain used for transformation. Rapeseed selection is normally performed using a selectable plant marker. Additionally, Agrobacterium mediated gene transfer to flax can be performed using, for example, a technique described by Mlynarova et al. (1994, Plant Cell Report 13:282-285).

The Arabidopsis KCS or KCS-like gene was cloned into a binary vector and expressed either under the seed specific USP (unknown seed protein) promoter (Baeumlein et al. 1991, Mol. Gen. Genetics 225:459-67). Alternatively, the PtxA promoter (the promoter of the Pisum sativum PtxA gene), which is a promoter active in virtually all plant tissues or the superpromoter, which is a constitutive promoter (Stanton B. Gelvin, U.S. Pat. No. 5,428,147 and U.S. Pat. No. 5,217,903) or other seed-specific promoters like the legumin B4 promoter (LeB4; Baeumlein et al. 1992, Plant J. 2:233-239) as well as promoters conferring seed-specific expression in monocot plants like maize, barley, wheat, rye, rice etc. were used.

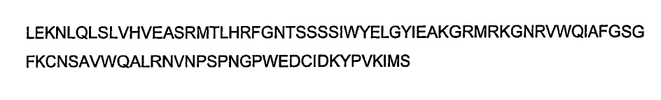

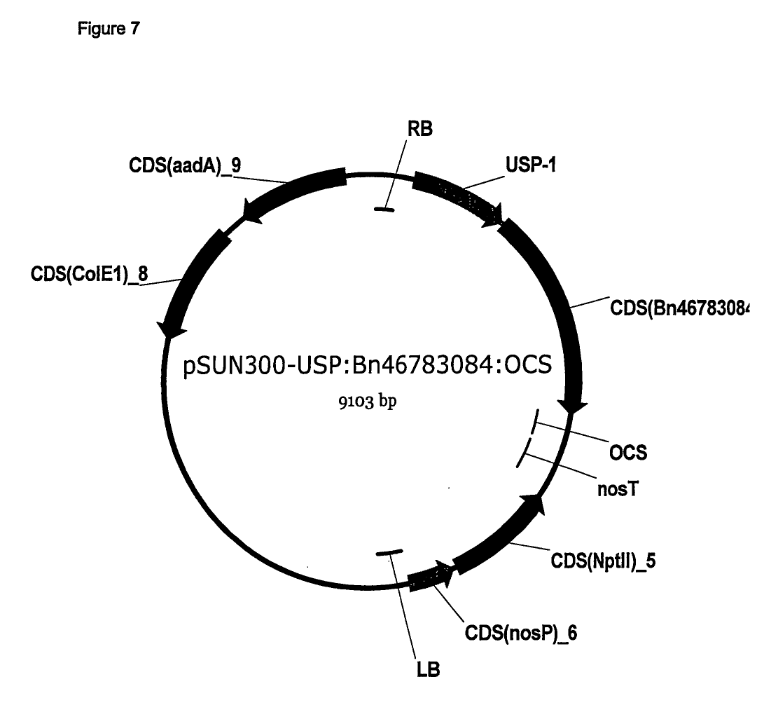

The nptII gene was used as a selectable marker in these constructs. FIG. 7 shows the scheme of a binary vector construct containing an KCS-like sequence from Brassica napus.

Transformation of soybean can be performed using for example a technique described in EP 0424 047, U.S. Pat. No. 5,322,783 (Pioneer Hi-Bred International) or in EP 0397 687, U.S. Pat. No. 5,376,543 or U.S. Pat. No. 5,169,770 (University Toledo), or by any of a number of other transformation procedures known in the art. Soybean seeds are surface sterilized with 70% ethanol for 4 minutes at room temperature with continuous shaking, followed by 20% (v/v) Clorox supplemented with 0.05% (v/v) tween for 20 minutes with continuous shaking. Then the seeds are rinsed 4 times with distilled water and placed on moistened sterile filter paper in a Petri dish at room temperature for 6 to 39 hours. The seed coats are peeled off, and cotyledons are detached from the embryo axis. The embryo axis is examined to make sure that the meristematic region is not damaged. The excised embryo axes are collected in a half-open sterile Petri dish and air-dried to a moisture content less than 20% (fresh weight) in a sealed Petri dish until further use.

The method of plant transformation is also applicable to Brassica napus and other crops. In particular, seeds of canola are surface sterilized with 70% ethanol for 4 minutes at room temperature with continuous shaking, followed by 20% (v/v) Clorox supplemented with 0.05% (v/v) Tween for 20 minutes, at room temperature with continuous shaking. Then, the seeds are rinsed 4 times with distilled water and placed on moistened sterile filter paper in a Petri dish at room temperature for 18 hours. The seed coats are removed and the seeds are air dried overnight in a half-open sterile Petri dish. During this period, the seeds lose approximately 85% of their water content. The seeds are then stored at room temperature in a sealed Petri dish until further use.

Agrobacterium tumefaciens culture is prepared from a single colony in LB solid medium plus appropriate antibiotics (e.g. 100 mg/A streptomycin, 50 mg/A kanamycin) followed by growth of the single colony in liquid LB medium to an optical density at 600 nm of 0.8. Then, the bacteria culture is pelleted at 7000 rpm for 7 minutes at room temperature, and re-suspended in MS (Murashige & Skoog 1962, Physiol. Plant. 15:473-497) medium supplemented with 100 mM acetosyringone. Bacteria cultures are incubated in this pre-induction medium for 2 hours at room temperature before use. The axis of soybean zygotic seed embryos at approximately 44% moisture content are imbibed for 2 h at room temperature with the pre-induced Agrobacterium suspension culture. (The imbibition of dry embryos with a culture of Agrobacterium is also applicable to maize embryo axes). The embryos are removed from the imbibition culture and are transferred to Petri dishes containing solid MS medium supplemented with 2% sucrose and incubated for 2 days, in the dark at room temperature. Alternatively, the embryos are placed on top of moistened (liquid MS medium) sterile filter paper in a Petri dish and incubated under the same conditions described above. After this period, the embryos are transferred to either solid or liquid MS medium supplemented with 500 mg/l carbenicillin or 300 mg/l cefotaxime to kill the agrobacteria. The liquid medium is used to moisten the sterile filter paper. The embryos are incubated during 4 weeks at 25° C., under 440 μmol m⁻²s⁻¹ and 12 hours photoperiod. Once the seedlings have produced roots, they are transferred to sterile metromix soil. The medium of the in vitro plants is washed off before transferring the plants to soil. The plants are kept under a plastic cover for 1 week to favor the acclimatization process. Then the plants are transferred to a growth room where they are incubated at 25° C., under 440 μmol m⁻²s⁻¹ light intensity and 12 h photoperiod for about 80 days.

Samples of the primary transgenic plants (T₀) are analyzed by PCR to confirm the presence of T-DNA. These results are confirmed by Southern hybridization wherein DNA is electrophoresed on a 1% agarose gel and transferred to a positively charged nylon membrane (Roche Diagnostics). The PCR DIG Probe Synthesis Kit (Roche Diagnostics) is used to prepare a digoxigenin-labeled probe by PCR as recommended by the manufacturer.

As an example for monocot transformation, the construction of ptxA promoter in combination with maize Ubiquitin intron and KCS-like nucleic acid molecules is described. The PtxA-KCS ortholog gene construct in pUC is digested with PacI and XmaI. pBPSMM348 is digested with PacI and XmaI to isolate maize Ubiquitin intron (ZmUbi intron) followed by electrophoresis and the QIAEX II Gel Extraction Kit (cat# 20021). The ZmUbi intron is ligated into the PtxA-KCS or KCS like nucleic acid molecule in pUC to generate pUC based PtxA-ZmUbi intron-KCS or KCS-like nucleic acid molecule construct followed by restriction enzyme digestion with AfeI and PmeI. PtxA-ZmUbi intron KCS or KCS-like gene cassette is cut out of a Seaplaque low melting temperature agarose gel (SeaPlaque® GTG® Agarose catalog No. 50110) after electrophoresis. A monocotyledonous base vector containing a selectable marker cassette (Monocot base vector) is digested with PmeI. The KCS or KCS-like nucleic acid molecule expression cassette containing ptxA promoter-ZmUbi intron is ligated into the Monocot base vector to generate PtxA-ZmUbi intron-KCS construct (see FIG. 22). Subsequently, the PtxA-ZmUbi intron-KCS or KCS-like nucleic acid molecule construct is transformed into a recombinant LBA4404 strain containing pSB1 (super vir plasmid) using electroporation following a general protocol in the art. Agrobacterium-mediated transformation in maize is performed using immature embryo following a protocol described in U.S. Pat. No. 5,591,616. An imidazolinoneherbicide selection is applied to obtain transgenic maize lines. In GUS expression experiments using the ptxA promoter::ZmUbi intron in maize strong expression was described in embryonic calli and roots (Song H-S. et al., 2004 PF 55368-2 US).

In general, a rice (or other monocot) KCS gene or KCS-like gene under a plant promoter like PtxA could be transformed into corn, or another crop plant, to generate effects of monocot KCS genes in other monocots, or dicot KCS genes in other dicots, or monocot genes in dicots, or vice versa. The plasmids containing these KCS or KCS-like coding sequences, 5′ of a promoter and 3′ of a terminator would be constructed in a manner similar to those described for construction of other plasmids herein.

### Example 12

### In Vivo Mutagenesis

In vivo mutagenesis of microorganisms can be performed by incorporation and passage of the plasmid (or other vector) DNA through E. coli or other microorganisms (e.g. Bacillus spp. or yeasts such as Saccharomyces cerevisiae) that are impaired in their capabilities to maintain the integrity of their genetic information. Typical mutator strains have mutations in the genes for the DNA repair system (e.g., mutHLS, mutD, mutT, etc.; for reference, see Rupp W. D. 1996, DNA repair mechanisms, in: Escherichia coli and Salmonella, p. 2277-2294, ASM: Washington.) Such strains are well known to those skilled in the art. The use of such strains is illustrated, for example, in Greener and Callahan 1994, Strategies 7:32-34. Transfer of mutated DNA molecules into plants is preferably done after selection and testing in microorganisms. Transgenic plants are generated according to various examples within the exemplification of this document.

### Example 13

### Assessment of the mRNA Expression and Activity of a Recombinant Gene Product in the Transformed Organism

The activity of a recombinant gene product in the transformed host organism can be measured on the transcriptional or/and on the translational level. A useful method to ascertain the level of transcription of the gene (an indicator of the amount of mRNA available for translation to the gene product) is to perform a Northern blot (for reference see, for example, Ausubel et al. 1988, Current Protocols in Molecular Biology, Wiley: New York), in which a primer designed to bind to the gene of interest is labeled with a detectable tag (usually radioactive or chemiluminescent), such that when the total RNA of a culture of the organism is extracted, run on gel, transferred to a stable matrix and incubated with this probe, the binding and quantity of binding of the probe indicates the presence and also the quantity of mRNA for this gene. This information at least partially demonstrates the degree of transcription of the transformed gene. Total cellular RNA can be pre-pared from plant cells, tissues or organs by several methods, all well-known in the art, such as that described in Bormann et al. (1992, Mol. Microbiol. 6:317-326).

To assess the presence or relative quantity of protein translated from this mRNA, standard techniques, such as a Western blot, may be employed (see, for example, Ausubel et al. 1988, Current Protocols in Molecular Biology, Wiley: New York). In this process, total cellular proteins are extracted, separated by gel electrophoresis, transferred to a matrix such as nitrocellulose, and incubated with a probe, such as an antibody, which specifically binds to the desired protein. This probe is generally tagged with a chemiluminescent or calorimetric label, which may be readily detected. The presence and quantity of label observed indicates the presence and quantity of the desired mutant protein present in the cell.

The activity of LMPs that bind to DNA can be measured by several wellestablished methods, such as DNA band-shift assays (also called gel retardation assays). The effect of such LMP on the expression of other molecules can be measured using reporter gene assays (such as that described in Kolmar H. et al. 1995, EMBO J. 14:3895-3904 and references cited therein). Reporter gene test systems are well known and established for applications in both prokaryotic and eukaryotic cells, using enzymes such as beta-galactosidase, green fluorescent protein, and several others.

The determination of activity of lipid metabolism membrane-transport proteins can be performed according to techniques such as those described in Gennis R. B. (1989 Pores, Channels and Transporters, in Biomembranes, Molecular Structure and Function, Springer: Heidelberg, pp. 85-137, 199-234 and 270-322).

### Example 14

### In Vitro Analysis of the Function of Brassica napus, Glycine max or Linum usitatissimum KCS and KCS-like Genes in Transgenic Plants

The determination of activities and kinetic parameters of enzymes is well established in the art. Experiments to determine the activity of any given altered enzyme must be tailored to the specific activity of the wild-type enzyme, which is well within the ability of one skilled in the art. Overviews about enzymes in general, as well as specific details concerning structure, kinetics, principles, methods, applications and examples for the determination of many enzyme activities may be found, for example, in the following references: Dixon, M. & Webb, E. C. 1979, Enzymes. Longmans: London; Fersht, (1985) Enzyme Structure and Mechanism. Freeman: New York; Walsh (1979) Enzymatic Reaction Mechanisms. Freeman: San Francisco; Price, N. C., Stevens, L. (1982) Fundamentals of Enzymology. Oxford Univ. Press: Oxford; Boyer, P. D., ed. (1983) The Enzymes, 3rd ed. Academic Press: New York; Bisswanger, H., (1994) Enzymkinetik, 2nd ed. VCH: Weinheim (ISBN 3527300325); Bergmeyer, H. U., Bergmeyer, J., Graβl, M., eds. (1983-1986) Methods of Enzymatic Analysis, 3rd ed., vol. I-XII, Verlag Chemie: Weinheim; and Ullmann's Encyclopedia of Industrial Chemistry (1987) vol. A9, Enzymes. VCH: Weinheim, p. 352-363.

### Example 15

### Analysis of the Impact of Recombinant Proteins on the Production of a Desired Seed Storage Compound

Seeds from transformed Arabidopsis thaliana plants were analyzed by gas chromatography (GC) for total oil content and fatty acid profile For this, total fatty acids were extracted with methyl tertiary-butyl ether (MTBE) and derivatised to their corresponding fatty acid methyl esters (FAMEs) with trimethylsulfonium hydroxide (TMSH). The separation of the FAMEs was performed on a capillary column (DB Wax 10 m×0.1 mm×0.2 μm) in an Agilent Technology 6890N Network GC System equipped with a flame ionization detector (FID) using hydrogen as carrier gas. Determination of the fatty acid content was performed by adding an internal standard of known concentration.

GC analysis reveals that Arabidopsis plants transformed with a construct containing USP promoter driving the Arabidopsis KCS (At1g19440) gene show an increase in total seed oil content by 13-18% compared with Columbia-0 in both segregating T2 and homozygous T3 seed generation (FIG. 8). The total seed protein level was virtually the same level as compared with a control plant (data not shown).

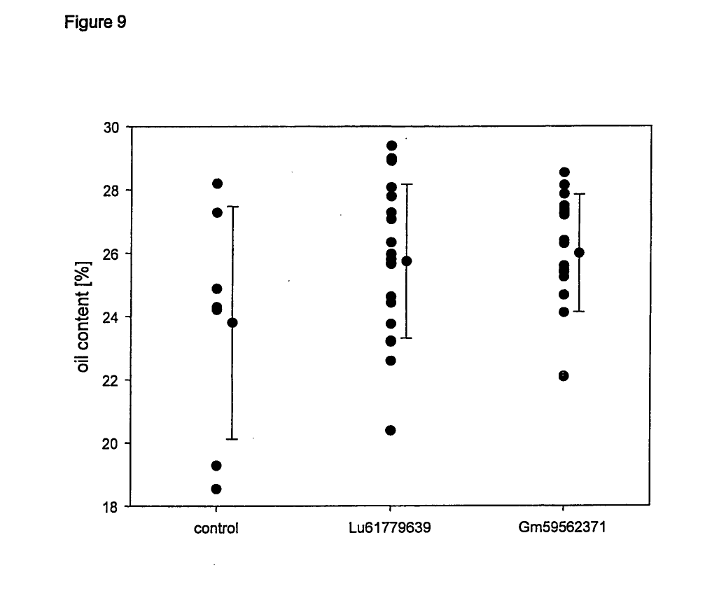

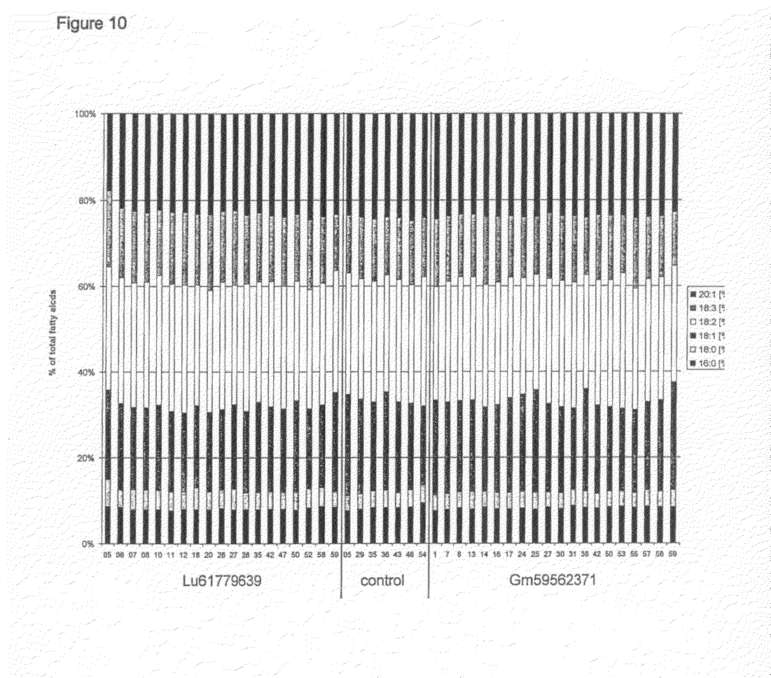

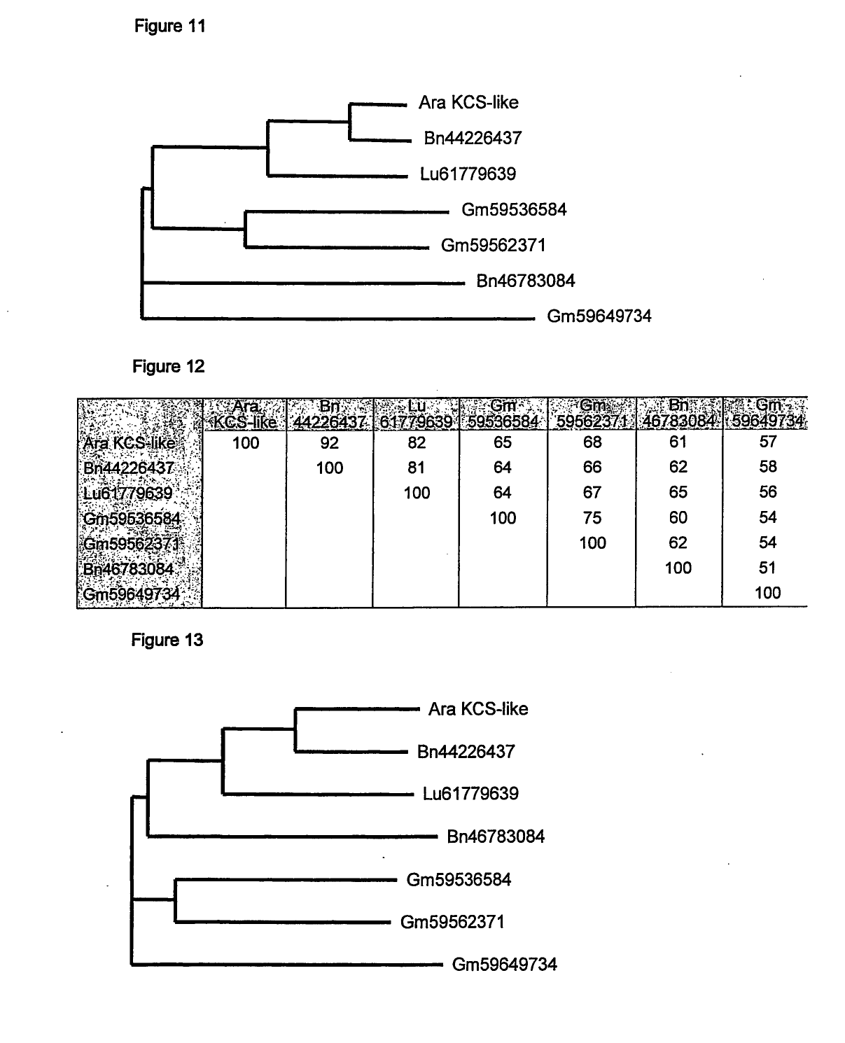

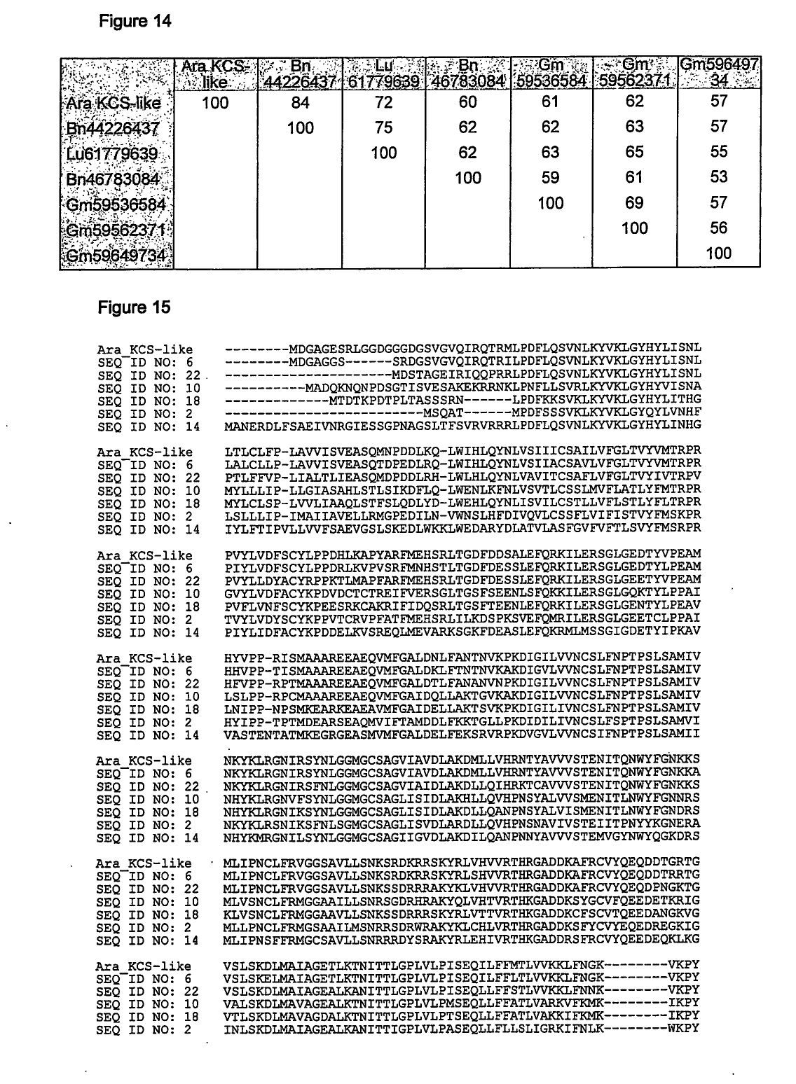

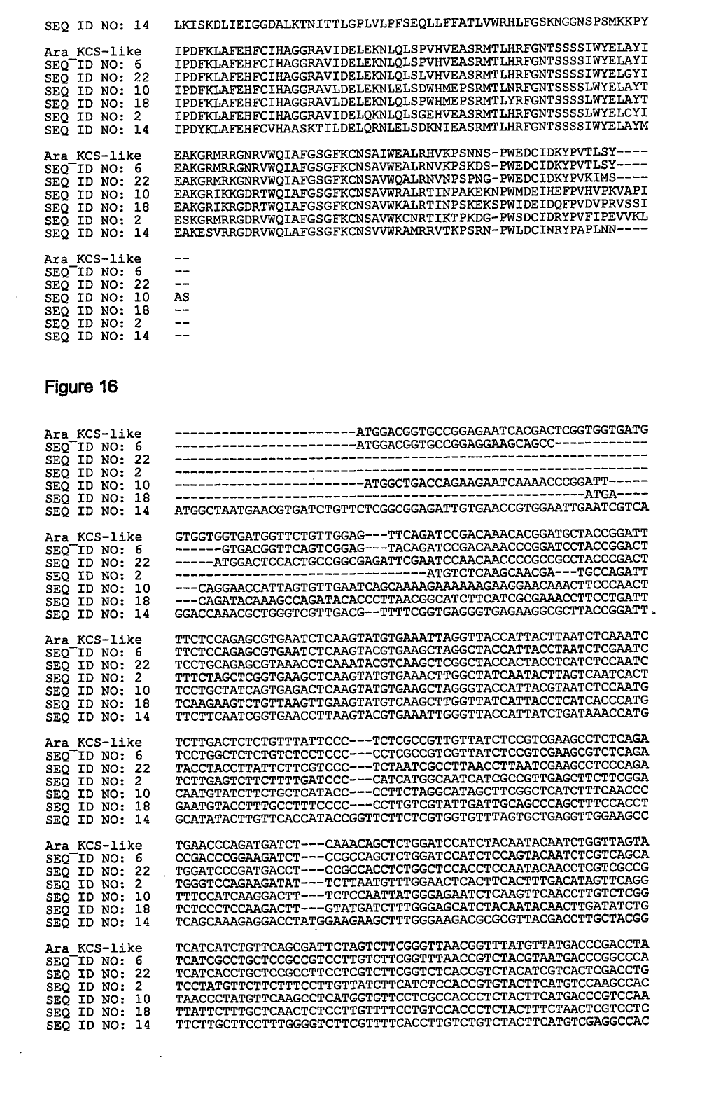

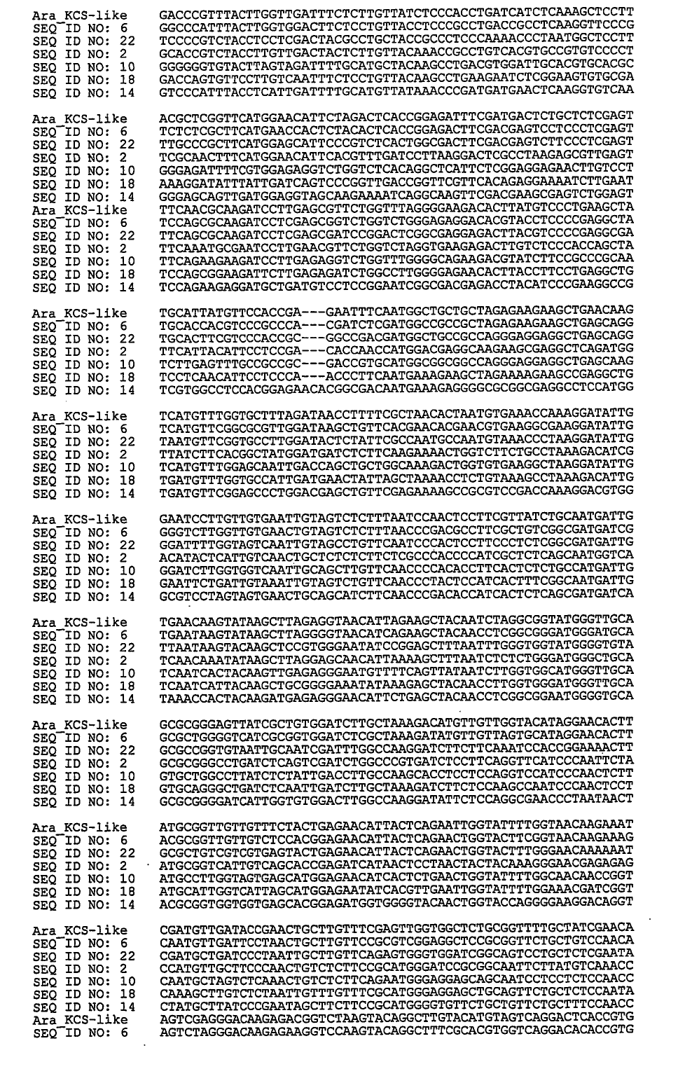

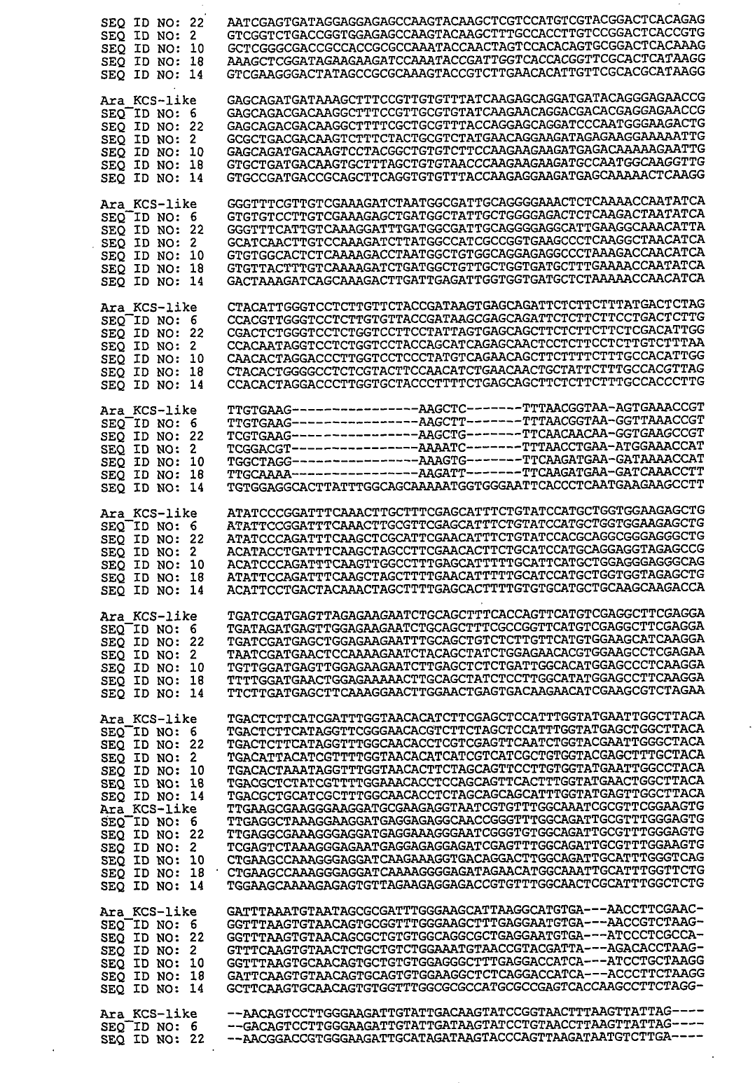

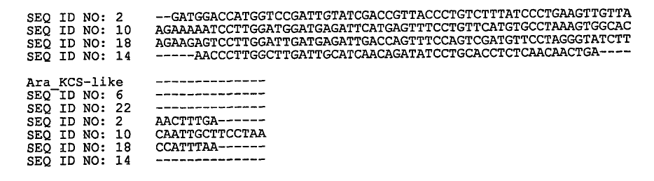

Furthermore, it was shown that seed specific overexpression of the orthologous KCS genes from Linum usitatissimum (61779639) and Glycine max (59562371) in Arabidopsis resulted in a relative oil increase in the segregating T2 seed of up to 21% and 16%, respectively, compared with plants transformed with the empty vector (FIG. 9). The oil increase was not accompanied by a change in the fatty acid profile (FIG. 10).

The results suggest that KCS overexpression with a promoter like USP allows the manipulation of total seed oil content.

The effect of the genetic modification in plants on a desired seed storage compound (such as a sugar, lipid or fatty acid) can be assessed by growing the modified plant under suitable conditions and analyzing the seeds or any other plant organ for increased production of the desired product (i.e., a lipid or a fatty acid). Such analysis techniques are well known to one skilled in the art, and include spectroscopy, thin layer chromatography, staining methods of various kinds, enzymatic and microbiological methods, and analytical chromatography such as high performance liquid chromatography (see, for example, Ullman 1985, Encyclopedia of Industrial Chemistry, vol. A2, pp. 89-90 and 443-613, VCH: Weinheim; Fallon, A. et al. 1987, Applications of HPLC in Biochemistry in: Laboratory Techniques in Biochemistry and Molecular Biology, vol. 17; Rehm et al., 1993 Product recovery and purification, Biotechnology, vol. 3, Chapter III, pp. 469-714, VCH: Weinheim; Belter, P. A. et al., 1988 Bioseparations: downstream processing for biotechnology, John Wiley & Sons; Kennedy J. F. & Cabral J. M. S. 1992, Recovery processes for biological materials, John Wiley and Sons; Shaeiwitz J. A. & Henry J. D. 1988, Biochemical separations in: Ulmann's Encyclopedia of Industrial Chemistry, Separation and purification techniques in biotechnology, vol. B3, Chapter 11, pp. 1-27, VCH: Weinheim; and Dechow F. J. 1989).

Besides the above-mentioned methods, plant lipids are extracted from plant material as described by Cahoon et al. (1999, Proc. Natl. Acad. Sci. USA 96, 22:12935-12940) and Browse et al. (1986, Anal. Biochemistry 442:141-145). Qualitative and quantitative lipid or fatty acid analysis is described in Christie, William W., Advances in Lipid Methodology. Ayr/Scotland:Oily Press.—(Oily Press Lipid Library; Christie, William W., Gas Chromatography and Lipids. A Practical Guide—Ayr, Scotland:Oily Press, 1989 Repr. 1992.—IX, 307 S.—(Oily Press Lipid Library; and “Progress in Lipid Research, Oxford:Pergamon Press, 1 (1952)-16 (1977) Progress in the Chemistry of Fats and Other Lipids CODEN.

Unequivocal proof of the presence of fatty acid products can be obtained by the analysis of transgenic plants following standard analytical procedures: GC, GC-MS or TLC as variously described by Christie and references therein (1997 in: Advances on Lipid Methodology 4th ed.: Christie, Oily Press, Dundee, pp. 119-169; 1998). Detailed methods are described for leaves by Lemieux et al. (1990, Theor. Appl. Genet. 80:234-240) and for seeds by Focks & Benning (1998, Plant Physiol. 118:91-101).

Positional analysis of the fatty acid composition at the sn-1, sn-2 or sn-3 positions of the glycerol backbone is determined by lipase digestion (see, e.g., Siebertz & Heinz 1977, Z. Naturforsch. 32c:193-205, and Christie 1987, Lipid Analysis 2^{nd} Edition, Pergamon Press, Exeter, ISBN 0-08-023791-6).

Total seed oil levels can be measured by any appropriate method. Quantitation of seed oil contents is often performed with conventional methods, such as near infrared analysis (NIR) or nuclear magnetic resonance imaging (NMR). NIR spectroscopy has become a standard method for screening seed samples whenever the samples of interest have been amenable to this technique. Samples studied include canola, soybean, maize, wheat, rice, and others. NIR analysis of single seeds can be used (see e.g. Velasco et al., ‘Estimation of seed weight, oil content and fatty acid composition in intact single seeds of rapeseed (Brassica napus L.) by near-infrared reflectance spectroscopy,’ Euphytica, Vol. 106, 1999, pp. 79-85). NMR has also been used to analyze oil content in seeds (see e.g. Robertson & Morrison, “Analysis of oil content of sunflower seed by wide-line NMR, “Journal of the American Oil Chemists Society, 1979, Vol. 56, 1979, pp. 961-964, which is herein incorporated by reference in its entirety).

A typical way to gather information regarding the influence of increased or decreased protein activities on lipid and sugar biosynthetic pathways is for example via analyzing the carbon fluxes by labeling studies with leaves or seeds using ¹⁴C-acetate or ¹⁴C-pyruvate (see, e.g. Focks & Benning 1998, Plant Physiol. 118:91-101; Eccleston & Ohlrogge 1998, Plant Cell 10:613-621). The distribution of carbon-14 into lipids and aqueous soluble components can be determined by liquid scintillation counting after the respective separation (for example on TLC plates) including standards like ¹⁴C-sucrose and ¹⁴C-malate (Eccleston & Ohirogge 1998, Plant Cell 10:613-621).

Material to be analyzed can be disintegrated via sonification, glass milling, liquid nitrogen and grinding or via other applicable methods. The material has to be centrifuged after disintegration. The sediment is re-suspended in distilled water, heated for 10 minutes at 100° C., cooled on ice and centrifuged again followed by extraction in 0.5 M sulfuric acid in methanol containing 2% dimethoxypropane for 1 hour at 90° C. leading to hydrolyzed oil and lipid compounds resulting in trans-methylated lipids. These fatty acid methyl esters are extracted in petrolether and finally subjected to GC analysis using a capillary column (Chrompack, WCOT Fused Silica, CP-Wax-52 CB, 25 m, 0.32 mm) at a temperature gradient between 170° C. and 240° C. for 20 minutes and 5 min. at 240° C. The identity of resulting fatty acid methylesters is defined by the use of standards available form commercial sources (i.e., Sigma).

In case of fatty acids where standards are not available, molecule identity is shown via derivatization and subsequent GC-MS analysis. For example, the localization of triple bond fatty acids is shown via GC-MS after derivatization via 4,4-Dimethoxy-oxazolin-Derivaten (Christie, Oily Press, Dundee, 1998).

A common standard method for analyzing sugars, especially starch, is published by Stitt M., Lilley R. Mc. C., Gerhardt R. and Heldt M. W. (1989, “Determination of metabolite levels in specific cells and subcellular compartments of plant leaves” Methods Enzymol. 174:518-552; for other methods see also Härtel et al. 1998, Plant Physiol. Biochem. 36:407-417 and Focks & Benning 1998, Plant Physiol. 118:91-101).

For the extraction of soluble sugars and starch, 50 seeds are homogenized in 500 μl of 80% (v/v) ethanol in a 1.5-mi polypropylene test tube and incubated at 70° C. for 90 min. Following centrifugation at 16,000 g for 5 min, the supernatant is transferred to a new test tube. The pellet is extracted twice with 50 μl of 80% ethanol. The solvent of the combined supernatants is evaporated at room temperature under a vacuum. The residue is dissolved in 50 μl of water, representing the soluble carbohydrate fraction. The pellet left from the ethanol extraction, which contains the insoluble carbohydrates including starch, is homogenized in 200 μl of 0.2 N KOH, and the suspension is incubated at 95° C. for 1 h to dissolve the starch. Following the addition of 35 μl of 1 N acetic acid and centrifugation for 5 min at 16,000 g, the supernatant is used for starch quantification.

To quantify soluble sugars, 10 μl of the sugar extract is added to 990 μl of reaction buffer containing 100 mM imidazole, pH 6.9, 5 mM MgCl₂, 2 mM NADP, 1 mM ATP, and 2 units 2 ml⁻¹ of Glucose-6-P-dehydrogenase. For enzymatic determination of glucose, fructose and sucrose, 4.5 units of hexokinase, 1 unit of phosphoglucoisomerase, and 2 μl of a saturated fructosidase solution are added in succession. The production of NADPH is photometrically monitored at a wavelength of 340 nm. Similarly, starch is assayed in 30 μl of the insoluble carbohydrate fraction with a kit from Boehringer Mannheim.

An example for analyzing the protein content in leaves and seeds can be found by Bradford M. M. (1976, “A rapid and sensitive method for the quantification of microgram quantities of protein using the principle of protein dye binding” Anal. Biochem. 72:248-254). For quantification of total seed protein, 15-20 seeds are homogenized in 250 μl of acetone in a 1.5-ml polypropylene test tube. Following centrifugation at 16,000 g, the supernatant is discarded and the vacuum-dried pellet is resuspended in 250 μl of extraction buffer containing 50 mM Tris-HCl, pH 8.0, 250 mM NaCl, 1 mM EDTA, and 1% (w/v) SDS. Following incubation for 2 h at 25° C., the homogenate is centrifuged at 16,000 g for 5 min and 200 ml of the supernatant will be used for protein measurements. In the assay, y-globulin is used for calibration. For protein measurements, Lowry DC protein assay (Bio-Rad) or Bradford-assay (Bio-Rad) is used.

Enzymatic assays of hexokinase and fructokinase are performed spectrophotometrically according to Renz et al. (1993, Planta 190:156-165), of phosphogluco-isomerase, ATP-dependent 6-phosphofructokinase, pyrophosphatedependent 6-phospho-fructokinase, Fructose-1,6-bisphosphate aldolase, triose phosphate isomerase, glyceral-3-P dehydrogenase, phosphoglycerate kinase, phosphoglycerate mutase, enolase and pyruvate kinase are performed according to Burrell et al. (1994, Planta 194:95-101) and of UDP-Glucose-pyrophosphorylase according to Zrenner et al. (1995, Plant J. 7:97-107).

Intermediates of the carbohydrate metabolism, like Glucose-1-phosphate, Glucose-6-phosphate, Fructose-6-phosphate, Phosphoenolpyruvate, Pyruvate, and ATP are measured as described in Hartel et al. (1998, Plant Physiol. Biochem. 36:407-417) and metabolites are measured as described in Jelitto et al. (1992, Planta 188:238-244).

In addition to the measurement of the final seed storage compound (i.e., lipid, starch or storage protein) it is also possible to analyze other components of the metabolic pathways utilized for the production of a desired seed storage compound, such as intermediates and side-products, to determine the overall efficiency of production of the compound (Fiehn et al. 2000, Nature Biotech. 18:1447-1161).

For example, yeast expression vectors comprising the nucleic acids disclosed herein, or fragments thereof, can be constructed and transformed into Saccharomyces cerevisiae using standard protocols. The resulting transgenic cells can then be assayed for alterations in sugar, oil, lipid or fatty acid contents.

Similarly, plant expression vectors comprising the nucleic acids disclosed herein, or fragments thereof, can be constructed and transformed into an appropriate plant cell such as Arabidopsis, soybean, rapeseed, rice, maize, wheat, Medicago truncatula, etc., using standard protocols. The resulting transgenic cells and/or plants derived there from can then be assayed for alterations in sugar, oil, lipid or fatty acid contents.

Additionally, the sequences disclosed herein, or fragments thereof, can be used to generate knockout mutations in the genomes of various organisms, such as bacteria, mammalian cells, yeast cells, and plant cells (Girke at al. 1998, Plant J. 15:3948). The resultant knockout cells can then be evaluated for their composition and content in seed storage compounds, and the effect on the phenotype and/or genotype of the mutation. For other methods of gene inactivation include U.S. Pat. No. 6,004,804 “Non-Chimeric Mutational Vectors” and Puttaraju et al. (1999, “Spliceosome-mediated RNA trans-splicing as a tool for gene therapy” Nature Biotech. 17:246-252).

### Example 16

### Purification of the Desired Product from Transformed Organisms

An LMP can be recovered from plant material by various methods well known in the art. Organs of plants can be separated mechanically from other tissue or organs prior to isolation of the seed storage compound from the plant organ. Following homogenization of the tissue, cellular debris is removed by centrifugation and the supernatant fraction containing the soluble proteins is retained for further purification of the desired compound. If the product is secreted from cells grown in culture, then the cells are removed from the culture by low-speed centrifugation and the supernate fraction is retained for further purification.

The supernatant fraction from either purification method is subjected to chromatography with a suitable resin, in which the desired molecule is either retained on a chromatography resin while many of the impurities in the sample are not, or where the impurities are retained by the resin, while the sample is not. Such chromatography steps may be repeated as necessary, using the same or different chromatography resins. One skilled in the art would be well-versed in the selection of appropriate chromatography resins and in their most efficacious application for a particular molecule to be purified. The purified product may be concentrated by filtration or ultrafiltration, and stored at a temperature at which the stability of the product is maximized.

There is a wide array of purification methods known to the art and the preceding method of purification is not meant to be limiting. Such purification techniques are described, for example, in Bailey J. E. & Ollis D. F. 1986, Biochemical Engineering Fundamentals, McGraw-Hill:New York).

The identity and purity of the isolated compounds may be assessed by techniques standard in the art. These include high-performance liquid chromatography (HPLC), spectroscopic methods, staining methods, thin layer chromatography, analytical chromatography such as high performance liquid chromatography, NIRS, enzymatic assay, or microbiologically. Such analysis methods are reviewed in: Patek et al. (1994, Appl. Environ. Microbiol. 60:133-140), Malakhova et al. (1996, Biotekhnologiya 11:27-32) and Schmidt et al. (1998, Bioprocess Engineer 19:67-70), Ulmann's Encyclopedia of Industrial Chemistry (1996, Vol. A27, VCH: Weinheim, p. 89-90, p. 521-540, p. 540-547, p. 559-566, 575-581 and p. 581-587) and Michal G. (1999, Biochemical Pathways: An Atlas of Biochemistry and Molecular Biology, John Wiley and Sons; Fallon, A. et al. 1987, Applications of HPLC in Biochemistry in: Laboratory Techniques in Biochemistry and Molecular Biology, vol. 17).

TABLE 1
Plant Lipid Classes

| | |
|---|---|
| Neutral Lipids | Triacylglycerol (TAG) |
| | Diacylglycerol (DAG) |
| | Monoacylglycerol (MAG) |
| Polar Lipids | Monogalactosyldiacylglycerol (MGDG) |
| | Digalactosyldiacylglycerol (DGDG) |
| | Phosphatidylglycerol (PG) |
| | Phosphatidylcholine (PC) |
| | Phosphatidylethanolamine (PE) |
| | Phosphatidylinositol (PI) |
| | Phosphatidylserine (PS) |
| | Sulfoquinovosyldiacylglycerol |

TABLE 2
Common Plant Fatty Acids

| | |
|---|---|
| 16:0 | Palmitic acid |
| 16:1 | Palmitoleic acid |
| 16:3 | Palmitolenic acid |
| 18:0 | Stearic acid |
| 18:1 | Oleic acid |
| 18:2 | Linoleic acid |
| 18:3 | Linolenic acid |
| γ-18:3 | Gamma-linolenic acid * |
| 20:0 | Arachidic acid |
| 20:1 | Eicosenoic acid |
| 22:6 | Docosahexanoic acid (DHA) * |
| 20:2 | Eicosadienoic acid |
| 20:4 | Arachidonic acid (AA) * |
| 20:5 | Eicosapentaenoic acid (EPA) * |
| 22:1 | Erucic acid |

* These fatty acids do not normally occur in plant seed oils, but their production in transgenic plant seed oil is of importance in plant biotechnology.

TABLE 3
A table of the putative functions of the KCS-like
LMPs (the full length nucleic acid sequences can
be found in Appendix A using the sequence codes)

| Seq | Sequence | | | ORF |
|---|---|---|---|---|
| ID | name | Species | Function | position |
| 1 | Bn46783084 | Brassica napus | beta-ketoacyl-CoA | 43-1536 |
| | | | synthase | |
| 5 | Bn44226437 | Brassica napus | beta-ketoacyl-CoA | 31-1563 |
| | | | synthase | |
| 9 | Gm59536584 | Glycine max | beta-ketoacyl-CoA | 220-1785 |
| | | | synthase | |
| 13 | Gm59649734 | Glycine max | beta-ketoacyl-CoA | 75-1682 |
| | | | synthase | |
| 17 | Gm59562371 | Glycine max | beta-ketoacyl-CoA | 153-1685 |
| | | | synthase | |
| 21 | Lu61779639 | Linum usitatis- | beta-ketoacyl-CoA | 22-1533 |
| | | simum | synthase | |

1. Those skilled in the art will recognize, or will be able to ascertain using no more than routine experimentation, many equivalents to the specific embodiments of the invention described herein. Such equivalents are intended to be encompassed by the claims to the invention disclosed and claimed herein.

1. An isolated polypeptide comprising three or more of the peptide sequences selected form the group consisting of:
a. LFX¹X²X³X⁴X⁵X⁶X⁷X⁸F, whereas X² is not M,
b. CYX⁹PX¹⁰X¹¹X¹²X¹³X¹⁴X¹⁵X¹⁶X¹⁷X¹⁸X¹⁹X²⁰X²¹X²²X²³X²⁴X²⁵X²⁶X²⁷X²⁸X²⁹X³⁰X³¹X³²X³³X³⁴X³⁵X³⁶FQ, whereas X¹⁷ is not Y and whereas X³² is not D and whereas X³⁴ is not A,
c. LKYVKLGyX³⁷YX³X³⁹X⁴⁰X⁴¹X⁴²X⁴³X⁴⁴X⁴⁵X⁴⁶X⁴⁷X⁴P, whereas X⁴⁸ is not F,
d. AFGSGFKCNSX⁴⁹X⁵⁰W, whereas X⁵⁰ is not I, and
e. X⁵¹X⁵²X⁵³X⁵⁴X⁵⁵FQX⁵⁶X⁵⁷X⁵⁸LX⁵⁹X⁶⁰SGLG, whereas X⁵¹ is not D and whereas X⁵³ is not A, and whereas X has the meaning of any amino acid if not defined elsewhise above.

2. An isolated nucleic acid encoding a protein containing the isolated polypeptide as claimed in claim 1.

3. An isolated polypeptide encoded by the isolated nucleic acid according to claim 2.

4. An isolated nucleic acid comprising a polynucleotide sequence selected from the group consisting of:
a. a polynucleotide sequence as depicted in SEQ ID NO: 1, SEQ ID NO: 3, SEQ ID NO:5, or SEQ ID NO:7;
b. a polynucleotide sequence encoding a polypeptide as depicted in SEQ ID NO: 2,
SEQ ID NO: 4, SEQ ID NO: 6, or SEQ ID NO: 8;
c. a polynucleotide sequence having at least 70% sequence identity with the nucleic acid of a) or b) above;
d. a polynucleotide sequence that is complementary to the nucleic acid of a) or b) above; and
e. a polynucleotide sequence that hybridizes under stringent conditions to nucleic acid of a) or b) above.

5. An isolated polypeptide encoded by the polynucleotide sequence as claimed in claim 4.

6. The isolated nucleic acid of claim 4, wherein the isolated nucleic acid encodes a polypeptide that functions as a modulator of a seed storage compound in microorganisms or plants.

7. The isolated polypeptide of claim 5, wherein the isolated polypeptide sequence functions as a modulator of a seed storage compound in microorganisms or plants.

8. An expression vector containing the nucleic acid of claim 4, wherein the nucleic acid is operatively linked to a promoter selected from the group consisting of a seed-specific promoter, a root-specific promoter, and a non-tissue-specific promoter.

9. A method of producing a transgenic plant having a modified level of a seed storage compound weight percentage compared to the wild type comprising,
i. introducing into a plant cell of an expression vector containing a nucleic acid, and
ii. generating from the plant cell the transgenic plant, wherein the nucleic acid encodes a polypeptide that functions as a modulator of a seed storage compound in the plant, and wherein the nucleic acid comprises a polynucleotide sequence selected from the group consisting of:
a. a polynucleotide sequence as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23;
b. a polynucleotide sequence encoding a polypeptide as depicted in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22 or SEQ ID NO: 24;
c. a polynucleotide sequence having at least 70% sequence identity with the nucleic acid of a) or b) above;
d. a polynucleotide sequence that is complementary to the nucleic acid of a) or b) above; and
e. a polynucleotide sequence that hybridizes under stringent conditions to nucleic acid of a) or b) above.

10. The method of claim 9, wherein the nucleic acid comprises a polynucleotide sequence having at least 90% sequence identity with a polynucleotide sequence selected from the group consisting of:
a. a polynucleotide sequence as depicted in SEQ ID NO: 1, SEQ ID NO: 3, SEQ ID NO: 5, or SEQ ID NO: 7, and
b. a polynucleotide sequence encoding a polypeptide as depicted in SEQ ID NO: 2. SEQ ID NO: 4. SEQ ID NO: 6, or SEQ ID NO: 8.

11. The method of claim 9, wherein the total seed oil content weight percentage is increased in the transgenic plant as compared to the wild type variety of the plant.

12. A method of modulating the level of a seed storage compound weight percentage in a plant comprising, modifying the expression of a nucleic acid in the plant, comprising:
i. introducing into a plant cell of an expression vector comprising a nucleic acid, and
ii. generating from the plant cell the transgenic plant, wherein the nucleic acid encodes a polypeptide that functions as a modulator of a seed storage compound in the plant wherein the nucleic acid comprises a polynucleotide sequence selected from the group consisting of:
a. a polynucleotide sequence as depicted in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21 or SEQ ID NO: 23;
b. a polynucleotide sequence encoding a polypeptide as depicted in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22 or SEQ ID NO: 24;
c. a polynucleotide sequence having at least 70% sequence identity with the nucleic acid of a) or b) above;
d. a polynucleotide sequence that is complementary to the nucleic acid of a) or b) above; and
e. a polynucleotide sequence that hybridizes under stringent conditions to nucleic acid of a) or b) above.

13. The method of claim 12, wherein the total seed oil content weight percentage is increased in the transgenic plant as compared to the wild type variety of the plant.

14. A transgenic plant made by the method of claim 9.

15. The transgenic plant of claim 14, wherein the total seed oil content weight percentage is increased in the transgenic plant as compared to a wild type variety of the plant.

16. The transgenic plant of claim 14, wherein the plant is selected from the group consisting of rapeseed, canola, linseed, soybean, sunflower, maize, oat, rye, barley, wheat, pepper, tagetes, cotton, oil palm, coconut palm, flax, castor, sugarbeet, rice and peanut.

17. A seed produced by the transgenic plant of claim 14, wherein the plant expresses the polypeptide that functions as a modulator of a seed storage compound and wherein the plant is true breeding for a modified level of seed storage compound weight percentage as compared to a wild type variety of the plant.

18. A transgenic plant made by the method of claim 12.

19. The transgenic plant of claim 18, wherein the total seed oil content weight percentage is increased in the transgenic plant as compared to a wild type variety of the plant.

20. The transgenic plant of claim 18, wherein the plant is selected from the group consisting of rapeseed, canola, linseed, soybean, sunflower, maize, oat, rye, barley, wheat, pepper, tagetes, cotton, oil palm, coconut palm, flax, castor, sugarbeet, rice and peanut.

21. A seed produced by the transgenic plant of claim 18, wherein the plant expresses the polypeptide that functions as a modulator of a seed storage compound and wherein the plant is true breeding for a modified level of seed storage compound weight percentage as compared to a wild type variety of the plant.
